(19) United States
(12) Patent Application Publication
(10) Pub. No.: US 2024/0139480 A1
(43) Pub. Date: May 2, 2024

(54) RETENTIVE DEVICES FOR GASTROINTESTINAL TRACT

(71) Applicant: Teva Pharmaceutical Industries Ltd., Tel Aviv (IL)

(72) Inventors: Avshalom BEN MENACHEM, Tel Aviv (IL); Ilan ZALIT, Tel Aviv (IL)

(21) Appl. No.: 18/548,915

(22) PCT Filed: Mar. 2, 2022
(86) PCT No.: PCT/IB2022/051815

§ 371 Date: Sep. 4, 2023

(30) Foreign Application Priority Data
Mar. 2, 2021 (EP) 21160270.1

(51) Int. Cl. A61M 31/00

(52) U.S. Cl. CPC A61M 31/002; A61M 2210/1053; A61M 2210/106

(57) ABSTRACT

A device for temporary residence at a predetermined location of the gastrointestinal tract of a subject is described. The device transfers from a closed configuration into an expanded configuration. The device is retained at the predetermined location and transfers from the expanded configuration into an emptying configuration in which the device moves past the predetermined location. The device enables localized medical methods in the gastrointestinal tract of the subject.

### CROSS REFERENCE TO RELATED APPLICATION

This application claims the benefit of European Application No. 21160270.1, filed Mar. 2, 2021, the entirety of which is incorporated by reference herein.

### TECHNOLOGICAL FIELD

The present disclosure relates generally to devices for temporary residence at a predetermined location of the gastrointestinal (GI) tract, and to methods of use thereof, for example for regio-specific release of a substance at a site of the GI tract or other medical purposes.

### BACKGROUND

The GI tract generally provides a therapeutic medium for an individual's body. At times, therapeutic drugs may need to be dispensed during a specified continuous period of time within a particular region of the GI tract, such as within the stomach, or within the small intestine or large intestine such as at the ileocecal valve (ICV), to cure or alleviate the symptoms of some medical conditions. However, locally dispensing therapeutic drugs for a sufficiently long period of time inside a human body (i.e., in GI), can be difficult and requires a device or mechanism (e.g., special platform) to stay or to be retained for sufficient time and release the therapeutic drug for a specified period of time. Such a device or mechanism also would need to be operated in a safe manner, in that the device or mechanism needs to physically enter and empty of the human body.

Dispensing therapeutic drugs within a particular region of the GI tract inside a human body for a sufficiently long period of time may be useful for enabling treatment of a disease, for example by releasing a medication directly in the vicinity of said particular region (also referred to as topical treatment) and/or for improving action of medications which have an improved absorption into the blood circulation.

Gastric retentive platforms/devices that may be implemented for drug delivery, i.e., as gastric retentive drug delivery systems and gastric retentive drug delivery dosage forms (GRDF) would be particularly useful for the delivery of drugs that are preferentially absorbed in the stomach, and/or have better solubility in stomach, and/or intended for local treatment of stomach and/or duodenum, and/or poorly soluble in alkaline pH or intestine, and/or those drugs that degrade in the colon or in the intestine, and/or those drugs that disturb colonic microbes, and/or those drugs with poor patient adherence, or drugs that have slow and or incomplete intestinal absorption, short biological half-life and a low therapeutic index, e.g., absorption is limited to upper small intestine (narrow absorption window) such as the duodenum and or jejunum.

Conversely, dispensing therapeutic drugs directly within the small intestine or large intestine inside a human body may be useful for enabling treatment of a disease by releasing a medication at the disease site and/or for improving action of medications which have an improved absorption at a specific site.

The GI tract may further be subjected to different diagnostic examinations. In such examinations, the “retentive-diagnosis” device is required to stay or to be retained in the particular region of the GI tract (such as stomach, the cecum, the ascending colon or small/large intestine) for sufficient time. Such retentive-diagnosis devices may be used in cooperation with imaging devices or other measurement devices, such or local measurement of pH, or motility.

A desired GRDF would be orally dosed, be retained in the stomach for a prolonged predictable period of time, especially in a patient in a fasted state, during which it would release the drug in a predefined manner, while after releasing a sufficient amount of drug, the device will for example, disintegrate or disassemble and be emptied out of the stomach and eventually emptied out of the body. When operating in the body, the device would be safe. The device would have to operate in a consistent way under variable human GI physiology (intra-inter patient variability in GI motility, GI forces, GI dimension, stomach pylorus size, change in fed state during the day, etc.).

In practice, however, there are many challenges in designing GRDF systems. Examples include ability to manufacture and scale up, use of biocompatible materials, sufficient drug loading capacity, small swallow size while having controlled retention capabilities in the stomach during fed and fasted states and providing a shelf life compatible with a medical product. Further challenges are safety while being resident in the stomach or intestines for long periods (e.g., having low pressure on stomach antral tissue, see Gregersen et al., “Mechanical properties in the human gastric antrum using B-mode ultrasonography and antral distension”, Am J Physiol Gastrointest Liver Physiol. 2002 283(2):G368-75), and including methods to control the emptying time from the stomach to enhance treatment as well as emergency expelling, etc., (e.g., see Tripathi et al., “Current State and Future Perspectives on Gastroretentive Drug Delivery Systems”, Pharmaceutics 2019. 11(4): 193).

As for topical treatment within the small intestine or the large intestine, an effective way to provide topical dispensing (i.e., local release) to the GI tract (and/or to a particular portion or section of the GI tract, for example a lower part of the small intestine, the ileocecal junction and the ascending colon) of a therapeutic drug to treat the diseased tissue in the GI tract would be desirable.

In practice, however, there are several challenges to such an approach. Methods typically employed to deliver drugs locally all have their own drawbacks. For example, the usefulness of formulations relying on pH-mediated release, including but not limited to enteric coated formulations, for example Asacol® (mesalamine delayed released tablet) may be limited by the high inter- and intra-patient variability of pH and microflora. The utility may be further limited in patient populations having highly variable GI motility (e.g., patients with ulcerative colitis (UC)), contributing to unpredictable transit times (times for transitioning from one portion of the GI tract to an adjacent portion), (e.g., see Brunner M. et al., “Gastrointestinal transit, release and plasma pharmacokinetics of a new oral budesonide formulation,” Br. J. Clin. Pharmacol. (2006) 61(1), pp. 31-38). Moreover, pH is dysregulated in ulcerative colitis patients, making pH-dependent drug delivery technologies less predictable. As for site specific absorption, an effective way to provide improved systemic therapeutic exposure may be in many cases achieved by a system capable to release drug in a continuous manner (e.g., OROS® technology). However, GI transit and change in conditions along different GI segments (viscosity, permeability, metabolic enzyme, pH etc.) may limit the platform's capability to provide the optimal systemic exposure. More specifically, Osmotic-controlled Release Oral delivery Systems (OROS®) are aimed at releasing at a constant rate a drug for an extended period of time in the GI tract. However, as the OROS® platform moves along the GI tract and releases the drug, it transits through different GI segments having different GI conditions (e.g., dissolution and permeability characteristics). Consequently, although the drug may be released in zero order, the resulting absorption and exposure in the system may be less than optimal. For example, for a drug that can be absorbed only in the small intestine, an OROS® system designed for a 12 hr constant rate of release, may have only 4-6 hr of exposure in the small intestine, because the average drug transit through the small intestine (absorption window) is about 4-6 hr. Thus, the platform's capability to provide improved systemic exposure is inherently limited.

Therefore, there remains a significant unmet medical need for local dispensing of therapeutic drugs in particular with the aim of:
a. Gastric topical treatment: improved treatment regimens for gastrointestinal diseases, including a need for regimens which can dispense therapeutics in the gastric region for prolonged time like the stomach, the pyloric region and the proximal duodenum, thereby reducing or avoiding the drawbacks of other forms of administration.
b. ICV topical treatment: improved treatment regimens for gastrointestinal diseases, such as inflammatory bowel disease (IBD) (e.g., see Harris M. S. et al., “Review article: delivery and efficacy of topical 5-aminosalicylic acid (mesalamine) therapy in the treatment of ulcerative colitis”, Aliment Pharmacol. Ther. 2011; 33: 996-1009), including a need for regimens which can dispense therapeutics to specific locations within the GI tract (and particularly at the ileocecal region: the lower part of the small intestine, the ileocecal junction and the ascending colon), thereby reducing or avoiding the drawbacks of other forms of administration.
c. Improved systemic therapeutic exposure, i.e., achieving desired systemic plasma concentrations having minimal fluctuations while enabling better efficacy and safety.

In addition, be it for the purpose of gastric topical treatment (i.e., drug release in the stomach), intestinal topical treatment (e.g., drug release at ICV), or other localized medical interventions (e.g., diagnosis, imaging, measurements, etc.), medical devices to be ingested and retained at a target location of the GI tract of the subject should be simple to use, so as to enable daily administration, and for example placed folded in simple container (e.g., capsule).

Furthermore, it would be desirable that such devices or platforms present a long shelf-life, in that they remain fully functional and present sufficient retention properties (e.g., structural integrity) even after a long storage following manufacturing (e.g., and encapsulation), and/or use less material, so as to facilitate from weekly to daily administration and incur less exposure of the subject to such ingested material.

Improvements in any of these directions would provide a significant contribution to the area of GI retentive devices and in the field of delivery of drugs to the GI system and drug delivery platforms/devices and dosage forms.

### GENERAL DESCRIPTION

The present disclosure notably provides devices, also referred to as platforms, for temporary residence at a predetermined location of the GI tract of a subject which alleviate at least in part the limitations of the prior art techniques. The present disclosure also provides methods of use of such devices, as well as oral dosage forms able to cooperate with such devices.

Any device according to the present disclosure is configured to transfer from a closed configuration into an expanded configuration. In addition, when the device is in the expanded configuration and positioned at the predetermined location of the GI tract, the device is configured to be retained at the predetermined location of the GI tract. The device comprises a biasing assembly for biasing the device in the expanded configuration, for example including or consisting of a resiliently deformable member. The device is further configured to transfer from the expanded configuration into an emptying configuration. The device is configured to move past the predetermined location of the GI tract in the emptying configuration, so as to be emptied from the GI tract of the subject. The biasing assembly, which may be a resiliently deformable member occupies minimal dead volume (free space within the device) and enables the device to hold a high percent (relative to the volume of the device) of API, for example up to about 30% or 40% or more.

The predetermined location may be the stomach or any location within the small intestine or the large intestine (sometimes broadly referred to as “the intestine” in the following, the expression thus covering both alternatives), for example the ICV. Thus, the device may be configured in the expanded configuration to be retained in the stomach of the subject, and to pass through the pyloric valve in the emptying configuration. Alternatively, the device may for example be configured in the expanded configuration to be retained at the ICV of the subject, and to pass through the ICV in the emptying configuration.

Any device according to the present disclosure may be configured for allowing chyme flow while the device is in the expanded configuration, positioned and retained at the predetermined location of the GI tract. In other words, chyme is allowed to flow through or by the device. In the intestinal retention case, the device is preferably configured for allowing chyme flow while the device is in the expanded configuration, positioned and retained at the predetermined location, for example the ICV, to avoid obstruction. In the stomach retention case also, the device may be configured for allowing chyme flow while the device is in the expanded configuration, positioned and retained in the stomach. This reduces obstruction risks of the pyloric valve.

An application is for specifically releasing a therapeutic substance at said predetermined location of the GI tract, for example in the stomach (i.e., gastric release), or within the intestinal tract (i.e., small intestine or colonic release).

Gastric release has great benefits for some therapeutic agents when used in various medical conditions:
a. Treatment of a gastric ulcer, or gastroesophageal reflux disease.
b. Treatment of stomach cancer or stomach infections.
c. Improved systemic therapeutic exposure by durable absorption and improved bioavailability, i.e., achieving systemic plasma concentrations having minimal fluctuations enabling better efficacy and safety.

Colonic release has great benefits for some therapeutic agents when used in various medical conditions:
a. Treatment of Inflammatory Bowel Disease (IBD) such as Ulcerative Colitis (UC). Indeed, the ileocecal junction and ascending colon are inflammation sites (especially in Pancolitis). An effective disease treatment may be achieved when effective local and/or topical exposure of the drug in the inflamed tissue is obtained (i.e., high local concentration and long exposure duration).
b. Treatment of colonic cancer or colonic infections.
c. Improved systemic therapeutic exposure by local absorption and improved bioavailability, i.e., achieving systemic plasma concentrations having minimal fluctuations enabling better efficacy and safety.
d. New administration site for drug delivery.

Another application is for diagnosis and/or imaging. In such a case, the devices may be used in cooperation with an external imaging device, and/or embed a camera and/or one or more measurement devices (such as one or more sensors for local measurement of pH and/or motility).

In addition, any device according to the present disclosure comprises a mechanism that improves shelf-life and/or allows using less material, in the form of a locking assembly and/or means for allowing the biasing assembly to transfer from a relaxed state into a strained state while the device is in the closed configuration. Due to such a mechanism, the device may be stored for a relatively long time after manufacturing (e.g., and encapsulation in a container), and still be fully functional when dosed to (e.g., ingested by) a subject so as to expand properly and be retained for a sufficient time at the predetermined location of the GI tract. Additionally or alternatively, such a mechanism allows using less material, and in particular less of the material constitutive of the biasing capability of the biasing assembly (e.g., silicone and/or metal). Thus, the subject intakes and is thus exposed to less such material. Furthermore, the use of less material allows achieving relatively small dimensions for the device, thus facilitating administration, and in particular ingestion. Thus, the mechanism enables a relatively high administration frequency, such as between weekly and daily administration, i.e., one or more times a day or one or more times a week. In some embodiments a less frequent administration is desired, for example, once every two weeks or once a month administration. In any case, the device achieves a high ratio of shelf-life duration or lifetime relative to quantity of material used and/or minimal size required for the device for a desired expansion force and a desired retention force.

Therefore, the present disclosure provides devices, oral dosage forms, and methods, according to any of the following aspects.

According to a first aspect, a device is provided for temporary residence at a predetermined location of the GI tract of a subject. The device is configured to transfer from a closed configuration into an expanded configuration. The device comprises at least two flexible arms, each arm having a first end and a second end. The device further comprises a first and second coupling heads, wherein the first end of each arm is coupled to the first coupling head and the second end of each arm is coupled to the second coupling head. The device further comprises a resiliently deformable member configured to force the coupling heads together to bend the arms. The resiliently deformable member thereby biases the device in the expanded configuration. The device further comprises a locking assembly configured, when activated, to maintain the device in the expanded configuration. Said locking assembly is configured to be activated upon transfer of the device into the expanded configuration. The device is configured in the expanded configuration, when the device is positioned at the predetermined location of the GI tract, to be retained at the predetermined location of the GI tract. The device is further configured to transfer from the expanded configuration into an emptying configuration. The device is configured to move past the predetermined location of the GI tract in the emptying configuration.

The coupling heads may come close one to another in the expanded configuration due to retraction of the resiliently deformable member, i.e., the resiliently deformable member forces the coupling heads to transit/move towards each other during transfer/deformation/transit of the device from the closed configuration into the expanded configuration, such that the coupling heads are closer one to another in the expanded configuration than in the closed configuration. Such deformation is allowed by the at least two flexible arms, which further impart expansion to the device, due to their bending. The device may further comprise a passage between the flexible arms when the flexible arms are bent, so as to allow chyme flow in the expanded configuration.

The locking assembly, when activated allows to lock and maintain the device in the expanded configuration, thus improving retention of the device at the predetermined location of the GI tract of the subject. The device thereby maintains dimensions forbidding it to move past the predetermined location of the GI tract. The resiliently deformable member allows to force and pull the coupling heads together so as to bend the flexible arms and thereby impart the expanded configuration to the device from the closed configuration. For that, the resiliently deformable member presents a certain tensile strength, while displaying resilience capacity, and is made of a certain type and quantity of material. The locking assembly is configured to be activated upon transfer of the device into the expanded configuration (and thus be inactive before such transfer). Thus, the resiliently deformable member is responsible for biasing and transferring the device into the expanded configuration, and the locking assembly is responsible for maintaining the device in the expanded configuration. The locking assembly may thus be said to form a “biasing shortening” assembly, in that it forms when activated a “shortening” or circumvention to the resiliently deformable member. When the locking assembly is activated, the resiliently deformable member may or may not force the coupling heads together, as the locking assembly retains anyway the coupling heads together at a fixed distance one from another (when the device is in expanded configuration, such that the device maintains an expanded shape), alone or in conjunction with the resiliently deformable member, so as to keep the flexible arms bent, thereby maintaining the device in the expanded configuration.

In particular, the unfolding force, that is, the force necessary to expand the device and perform the locking (i.e., activate the locking assembly) may be lower (e.g., significantly, for example at least twice or at least three times) than the emptying/retention force, that is, the force necessary to perform unlocking (i.e., deactivate/release the locking assembly) and compact the device. As a result, the resiliently deformable member may be enhanced for its main function, that is, the expanding function (also referred to as “unfolding” function), while the locking assembly may take over when it comes to the retention function. This enables relatively long shelf-life and/or low amount of material used for the resiliently deformable member. In addition, as retention is not dependent on the resiliently deformable member, a relatively high emptying or retention force may be achieved while keeping the unfolding force low. The locking assembly thus allows to take into account the natural creep phenomenon occurring for the resiliently deformable member during shelf-storage, that is, the reduction of its functionality over time due to material wear.

Furthermore, the locking assembly increases safety during dosing of the device if ingested by the subject, in case the device is unwillingly about to expand while in the esophagus. If the expansion/unfolding force of the device provided by the resiliently deformable member were very high (e.g., above 4.90 N, as determined in tests which were performed) to account for the creep phenomenon and/or because the expansion/unfolding force would be dependent of the retention force, then the expansion would be at risk of overcoming esophagus tissue constriction (pressing on the device to resist unfolding) such that the device would possibly overcome the esophagus tissue constriction and expand/unfold in the esophagus. The separation of the unfolding and retention functions allows keeping a relatively low unfolding force, provided by the resiliently deformable member, so as to reduce risks of unfolding while in the esophagus and to reduce material use by the resiliently deformable member, while achieving a relatively high retention force, provided by the locking assembly, so as to resist GI forces exerted at times on the device while retained at the predetermined location of the GI tract. Still, even if lower than the retention force and not high enough to resist all GI forces that may be exerted at times on the device, the unfolding force may be high enough to allow expansion of the device at an expected time along the GI tract, thus enough to activate the locking assembly which then takes over.

Optional features of the device according to the first aspect are now discussed.

In examples, in the closed configuration, the arms may be substantially straight, and/or the resiliently deformable member may be either stretched or stretchable, and either connected or connectable to the two coupling heads, so as to be able to pull/force the two coupling heads together and deform the device into the expanded configuration. In the expanded configuration, the arms are bent, and the resiliently deformable member may be either relaxed or stretched, but in such a case less than in the closed configuration. In other words, an extension of the resiliently deformable member may be larger in the closed configuration than in the expanded configuration and the arms may be substantially straight in the closed configuration. Optionally, in the manufacturing process, the resiliently deformable member may be reversibly stretched, thereby straightening the at least two arms to reversibly set the device into the closed configuration. Optionally, the locking assembly may be configured for locking the device substantially irreversibly in the expanded configuration. In such a case, once deformed into the expanded configuration, the device cannot return into the closed configuration unless breaking a part of the locking assembly. In other words, the locking assembly may be substantially rigid. Thus, only the emptying configuration allows expelling the device. Alternatively, the locking assembly may present a certain resilience, such that the device may return into or toward the closed configuration from the expanded configuration, e.g., provided that a sufficient emptying force is applied. In such a case, the locking assembly is configured for such sufficient emptying force to be significantly higher than GI forces, as the locking assembly is configured to resist to such GI forces and maintain the device in the expanded configuration when positioned at the predetermined location.

In examples, the locking assembly may be activated by snapping (i.e., snap-fitting) one or more part of the locking assembly each with a respective cooperating part of the device. The snapping may be such that the force required for performing the snapping is lower (e.g., substantially, e.g., at least twice or three times) than the force required to perform the unsnapping. A snapping activation allows a simple manufacturing. The snapping or snap-fitting may for example comprise one or more cantilever snap-fits. Alternatively, the locking assembly may be activated by a latching mechanism. For example, one or more parts of the locking assembly may each engage with a respective cooperating part of the device, and a latch may be actuated (i.e., moved) upon the engagement and fixedly attach the cooperating parts. A latching mechanism allows to achieve a particularly high retention.

In examples, the locking assembly may comprise a locking member arranged between the two coupling heads and having two ends. At least one end (e.g., only one end or both ends) of the locking member is free in the closed configuration. In other words, said at least one end is free or unattached from a respective coupling head with which said at least one end is to cooperate. Yet in other words, a distance separates said at least one end and its respective coupling head, such that they are disconnected. Further, the locking member is configured for each such free end to engage (i.e., come into contact with) its respective coupling head when the device transfers from the closed configuration into the expanded configuration, thereby attaching (i.e., fixedly connecting) the end to its respective coupling head. This allows to efficiently make use of the force used to pull the two coupling heads together to perform the locking. The case of only one free end for the locking member facilitates activation of the locking assembly.

Optionally in such examples, the locking member may be arranged longitudinally between the two coupling heads and/or along the resiliently deformable member (e.g., thus also arranged between the two coupling heads, for example also longitudinally). Optionally, the locking member may surround at least a (e.g., longitudinal) portion of the resiliently deformable member, at least partially on the periphery of said portion. This allows compactness of the device.

Additionally or alternatively in such examples, the locking member may be made of a more rigid and/or less elastic material than the resiliently deformable member. The locking member may notably present a higher tensile strength, a higher tensile modulus, and/or a lower elongation percentage at break, compared to the resiliently deformable member. For example, the locking member may be made of a rigid or semi-rigid material, such as cellulose acetate, Polyether ether ketone (PEEK), or polyurethane, or other biocompatible materials such as polylactic (PLA), polyglycolic (PLG) polymers.

Additionally or alternatively in such examples, the locking member may be shock-absorbing. This reduces risks of damage to the body tissue of the GI tract, by providing some resilience to the expanded device. The material composition and/or the structure design of the locking member may further convey a shock absorption capability to the device when exposed to GI forces in the expanded configuration. Such shock absorption capability makes the device more compliant to GI tissue, thus increasing safety of use, and it increases the durability of the device, e.g., during long GI retention.

Additionally or alternatively in such examples, the respective coupling head of at least one (e.g., each) free end of the locking member may comprise a recess configured for receiving the free end of the locking member and attaching said end to said respective coupling head when the device transfers from the closed configuration into the expanded configuration. For instance, the recess may be configured for snapping the free end of the locking member therein. This facilitates the locking of the device in the expanded configuration. Also, the snapping (e.g., cantilever snap-fit) may lock the free of the locking member, meaning that the force necessary to unsnap the locking member is higher (e.g., significantly, e.g., at least twice or three times higher) than the force necessary to perform the snapping. The snapping may be effective due to the resiliently deformable member pulling the two coupling heads together at a sufficient speed and/or with a sufficient force.

Optionally, at least one (e.g., each) free end of the locking member may have an anchor shape and is configured to be forced (e.g., snapped) into its respective coupling head, the coupling head being configured for retaining the free end afterwards. By “anchor shape”, it is meant any generally longitudinal shape with one or more radial protrusions at its extremity, for example a peripheral radial protrusion or one or more radial protrusions peripherally distributed. For instance, such an anchor shape may be formed by locking pins and/or locking dents protruding radially from the locking member. In turn, each coupling head configured for receiving in a recess such an anchor shape may comprise a corresponding shape in the recess cooperating with the radial protrusions of the anchor shape, for example radial recesses and/or protrusion, to achieve snap-fitting. The anchor shape allows that the force necessary to perform the locking of the locking member's free end(s) in the respective coupling head(s) be lower than the force necessary to unlock said free end(s). In other words, the anchor shape enables snapping. In addition, at least one (e.g., each) free end of the locking member may have a rounded extremity so as to be more easily forced into its respective coupling head. In addition, at least one (e.g., each) free end of the locking member and/or the receiving recess of the respective coupling head may be deformable so as to be reduced in diameter (i.e., dimension across a longitudinal snapping axis) during the snapping, so as to facilitate the snapping. The anchor shape and/or the shape of the receiving recess may be such that the deformation only occurs in one direction along the snapping axis, i.e., in the snapping direction, while in the opposite direction, the anchor shape and the recess cooperate such that one retains the other, for example by the retaining edges (e.g., generally orthogonal to the snapping axis).

In examples, the closed configuration has a storage sub-configuration (i.e., a certain configuration provided for shelf-storage of the device, e.g., after encapsulating and, optionally, even after packaging and in such a case before removal from a packaging of the device) and a dosing sub-configuration (i.e., a certain configuration provided for when the device is dosed to or ingested by the subject, e.g., after removal from a packaging of the device). The resiliently deformable member has two ends each configured to pull a respective coupling head. At least one end (e.g., only one end or both ends) of the resiliently deformable member is free in the storage sub-configuration. In other words, said at least one end is free or unattached from a respective coupling head with which said at least one end is to cooperate. Yet in other words, a distance separates said at least one end and its respective coupling head, such that they are disconnected. Thus, the resiliently deformable member is relaxed, that is, not fully stretched (i.e., at least to a lesser point than if it were attached to the two coupling heads on both ends). In addition, the resiliently deformable member is configured for being stretched and for each free end to be attached to its respective coupling head while the device is in the storage sub-configuration, thereby transferring the device into the dosing sub-configuration. In other words, the device is configured for transferring the resiliently deformable member from a relaxed state into a strained state, i.e., for imparting a certain amount of (e.g., additional) straining to the resiliently deformable member. This straining may also be referred to as “cocking” the device, as it amounts to supply it with potential energy. Such a “straining/cocking before dosing” capability of the device allows longer shelf-life, as it preserves the functionality of the resiliently deformable member during storage (since it is relaxed during storage, and since, if it otherwise were strained during storage, it would see its mechanical capabilities diminishing over time due to material creep). The stretching of the resiliently deformable member may be performed while the device stays in the closed configuration, thus while maintaining the locking assembly inactive (e.g., keeping the locking member's one or two free ends unattached from the respective one or two coupling heads). The locking assembly may thus be inactive even when the resiliently deformable member is in the strained state. The case of only one free for the resiliently deformable member end facilitates straining of the device.

Optionally in such examples, in the storage sub-configuration, the resiliently deformable member may be configured to be stretched by pulling each free end via at least one respective lead.

Such a lead may thus be part of a straining assembly for straining the device. The lead may be connected to the free end, and/or protrude out of the coupling head respective to the free end of the resiliently deformable member. Optionally, the respective coupling head of each free end of the resiliently deformable may comprise a recess for receiving the free end of the resiliently deformable member and attaching said end to said respective coupling head when the device transfers from the storage sub-configuration into the dosing sub configuration. Such a recess may be in communication with a recess receiving a free end of the locking member, both recesses thus forming together a locking tunnel. In other words, in such a case, the locking member and the resiliently deformable member each have a respective free end, configured to be attached to a same coupling head via said locking tunnel. The lead may protrude out of said locking tunnel. Further optionally, at least one (e.g., each) free end of the resiliently deformable member may have an anchor shape and be configured to be forced (e.g., snapped, or, deformed and press-fitted) into its respective coupling head, the coupling head being configured for retaining the free end afterwards. For instance, such an anchor shape may be formed by one or more arms of the resiliently deformable member extending radially from a longitudinal body of the resiliently deformable member, optionally distributed radially, or by one peripheral radial plate, e.g., and integrally formed with said longitudinal body of the resiliently deformable member. The recess may have an opening configured for forcing the free end therein, e.g., and the recess may be shaped with one or more tunnels each for fitting a respective arm of the optional anchor shape or a peripheral cavity for fitting the plate.

The respective lead may be configured to be attached to a packaging, such as a blister, such that when removing the device out of the packaging, the respective lead pulls its respective free end of the resiliently deformable member. Thus, by simply removing the device from its packaging, the subject (user) strains at the same time the resiliently deformable member and attaches any free end thereof to the respective coupling head, such that the device is now capable of transferring into the expanded configuration. This provides high simplicity of use to the subject, who does not need to worry about putting the device into the dosing sub-configuration.

The respective lead may be attached to the packaging by one of its ends or by both ends. When pulling the device out of the packaging, the lead remains attached, and thereby pulls the free end of the resiliently deformable member to stretch the resiliently deformable member. As the subject continues to pull, the free end of the resiliently deformable member becomes locked into its respective coupling head, such that further pulling the lead may cause detachment of the lead from the device. Thus, the lead is not (e.g., fully) ingested.

The device may implement different options for such detachment. In an option, at least one (e.g., each) free end configured to be pulled via a respective lead may comprise a tunnel (e.g., anchor tunnel) for threading the lead therein. This facilitates the manufacturing. Additionally or alternatively, the lead may be detachable from the packaging (e.g., blister). For example, the lead may have its two ends initially attached to the packaging in the storage sub-configuration, and after straining the resiliently deformable member, by continuing to pull the device out, at least one end (e.g., one or both ends) of the lead may be detached from the packaging. Since the lead is not fixed to the (initially free) end of the resiliently deformable member, but rather loosely threaded in a tunnel thereof, the lead may at this time be detached and separate from the resiliently deformable member. In another option, the respective lead may have one or more weak points for cutting the lead after the free end is attached to its respective coupling head and the lead continues to be pulled. In yet another option, at least one free end of the resiliently deformable member may comprise an extension configured for protruding out of its respective coupling head after said end is attached to its respective coupling head. In such a case, the resiliently deformable member may comprise a cutting zone at the basis of the extension for cutting the extension after said end is attached to its respective coupling head and the lead continues to be pulled. These options allow a simple removal of the lead after use by the subject before dosing the device.

Alternatively, each end of the resiliently deformable member may be attached to its respective coupling head in the storage sub-configuration, such that in the storage sub-configuration. In other words, the resiliently deformable member is already sufficiently strained and capable of transferring the device from the closed configuration into the expanded configuration during shelf-storage, e.g., substantially from manufacturing. It is said in this case that the device presents no “straining before dosing” capability. This facilitates the implementation of several support elements of a trigger assembly (such as presented later), and in particular several support elements each disabled by a different activation signal (e.g., one passive exposure signal and one active fast-disabling signal, as presented later). Indeed, each end of the resiliently deformable member may optionally in such a case be secured by (e.g., embedded in or press-fitted by) a respective support element arranged inside a respective coupling head. The device may further optionally in such a case comprise a locking element (not to be confused with (the parts of or the parts in action with) the locking assembly or the locking member thereof), such as a container (e.g., a capsule), arranged to temporary maintain the device in the closed configuration configured to release the device only at some point after dosing.

In examples, the device may be configured to transfer from the expanded configuration into the emptying configuration upon the locking assembly being deactivated (i.e., transferred from an active state into an inactive state). For example, in case the locking assembly comprises a locking member, the deactivation of the locking assembly may comprise or consist in detaching at least one end of the locking member from its respective coupling head, thereby releasing the constraint operated by the locking member. In any case where the locking assembly is deactivated, the device may be so configured that the resiliently deformable member (which is not shortened anymore) now forces again the coupling heads together, but insufficiently to resist to GI forces. As a result, the device is said to be in its emptying configuration, as it is not capable of being retained at the predetermined location and may thus be emptied. Alternatively, the device may be configured such that, upon the locking assembly being deactivated, the resiliently deformable member is prevented from forcing the coupling heads together at all, thereby allowing transfer into the emptying configuration. For example, the device may be configured such that, upon the locking assembly being deactivated, at least one end of the resiliently deformable member is detached from its respective coupling head. In particular examples where the locking assembly comprises a locking member and the deactivation of the locking assembly comprises or consists in detaching at least one end of the locking member from its respective coupling head, the at least one end of the resiliently deformable member also detached may be the one initially attached to the same respective coupling head. For instance, both said end of the locking member and said end of the resiliently deformable member may be secured to said respective coupling head by a respective support element (such as any one presented later), for example arranged inside said respective coupling head. In examples, the locking member and the resiliently deformable may both be detachable at each of their two ends (such as any one presented later) by a respective common support element arranged in the respective coupling head. The support elements in the two ends may be triggered by a different activation signal (such as any one presented later).

According to a second aspect, a device is provided for temporary residence at a predetermined location of the GI tract of a subject. The device is configured to transfer from a closed configuration into an expanded configuration. The device is further configured, in the expanded configuration, when the device is positioned at the predetermined location of the GI tract, to be retained at the predetermined location of the GI tract. The device is further configured to transfer from the expanded configuration into an emptying configuration, the device being configured to move past the predetermined location of the GI tract in the emptying configuration. The device further comprises a biasing assembly having a strained state and a relaxed state, the biasing assembly being configured, while the device is in the closed configuration, to transfer from the relaxed state into the strained state, the biasing assembly being in the strained state capable of transferring the device from the closed configuration into the expanded configuration.

In other words, the device further comprises a biasing assembly having a first state and a second state, the biasing assembly being more strained in the first state than in the second state, the biasing assembly being configured, while the device is in the closed configuration, to transfer from the second state into the first state, the biasing assembly being in the first state capable of transferring the device from the closed configuration into the expanded configurations.

When the biasing assembly is in the strained state, the biasing assembly can transfer the device from the closed configuration into the expanded configuration. In other words, the biasing assembly has the right amount of potential or mechanical energy for that. Conversely, when the biasing assembly is in the relaxed state, the biasing assembly may be incapable of transferring the device from the closed configuration into the expanded configuration, as it may have insufficient potential energy in such a case. Thus, the biasing assembly is more strained in the strained state than in the relaxed state. The biasing assembly in the relaxed state may be completely unstrained and have no potential energy at all, or alternatively it may be strained to some extent but with insufficient energy to be capable of forcing the two coupling heads together. Transferring from the relaxed state into the strained state thus imparts a certain amount of (e.g., additional) straining to the biasing assembly. By “capable of transferring the device from the closed configuration into the expanded configuration”, it is meant that the biasing assembly in itself has the right functionality and energy, but it is understood that other means may generally prevent such transfer into the expanded configuration and may allow it only at the right time. For example, the device may comprise a locking element, such as a container (e.g., a capsule), arranged to temporary maintain the device in the closed configuration configured to release the device only at some point after dosing.

The relaxed state of the biasing assembly may define a storage sub-configuration of the device, that is, a sub-configuration of the closed configuration wherein the device is to be shelf-stored. The strained state of the biasing assembly may in turn define a dosing sub-configuration of the device, that is, a sub-configuration of the closed configuration just before the device is to be dosed to the subject. As in the corresponding examples according to the first aspect, the straining may also be referred to as “cocking” the device, as it amounts to supply it with potential energy. Such a “cocking/straining before dosing” capability of the device allows longer shelf-life, as it preserves the functionality of the biasing assembly during storage (since it is relaxed during storage, and since, if it otherwise were strained during storage, it would see its mechanical capabilities diminishing over time due to material creep). In turn, as in the device according to the first aspect, a longer shelf-life allows a relatively low amount of material used for the biasing assembly. Thus, the subject intakes and is exposed to less material.

The device according to the second aspect may comprise at least two flexible arms (e.g., three or four flexible arms), each arm having a first end and a second end. The device may further comprise a first and second coupling heads, wherein the first end of each arm is coupled to the first coupling head and the second end of each arm is coupled to the second coupling head. The device may further comprise a resiliently deformable member configured to force the coupling heads together to bend the arms thereby biasing the device in an expanded configuration (e.g., wherein the coupling heads may come close one to another in the expanded configuration due to retraction of the resiliently deformable member, i.e., the resiliently deformable member forces the coupling heads to transit/move towards each other during transfer/deformation/transit of the device from the closed configuration into the expanded configuration, such that the coupling heads are closer one to another in the expanded configuration than in the closed configuration).

The device according to the second aspect may comprise a locking assembly according to the first aspect. Besides, the device according to the second aspect may comprise any other feature of the device according to the first aspect. Thus, a device according to the second aspect may also be according to the first aspect. Alternatively, the device according to the second aspect may comprise no such locking functionality, and the biasing assembly performs (e.g., fully) both the expanding function and the retention function. In such a case, the biasing assembly (e.g., resiliently deformable member) may be configured to continue forcing the two coupling heads together when the device has transferred into the expanded configuration, so as to maintain the arms bent and keep device in the expanded configuration.

Optional features of the device according to the second aspect are now discussed.

In examples, the device may comprise a straining assembly configured for transferring the biasing assembly from the relaxed state into the strained state upon the device being removed from a packaging. Thus, by simply removing the device from its packaging, the subject strains at the same time the biasing assembly, such that the device is now capable of transferring into the expanded configuration. This provides high simplicity of use to the subject, who does not need to worry about putting the device into the dosing sub-configuration.

For instance, the straining assembly may comprise a lead configured to be attached to a packaging, such as a blister, such that when removing the device out of the packaging, the lead is pulled and transfers the biasing assembly from the relaxed state into the strained state. The lead may be initially attached to the device (e.g., in the relaxed state or storage sub-configuration, that is, before activation of the straining assembly) and configured to be detached from the device (e.g., in the strained state or dosing sub-configuration, that is, after activation of the straining assembly). Thus, the lead is not (e.g., fully) ingested.

The device may implement different options for such detachment. In an option, the lead may have one or two ends attached to the packaging and the lead be detachable from the packaging from one or two of its attached ends. Additionally or alternatively, the lead may be threaded into a part of the device, e.g., an anchor tunnel. For example, the lead may have its two ends initially attached to the packaging in the storage sub-configuration, and after straining the biasing assembly, by continuing to pull the device out, at least one end (e.g., one or both ends) of the lead may be detached from the packaging. Since the lead is not fixed to the device, but rather loosely threaded in a tunnel thereof, the lead may at this time be detached and separate from the biasing assembly. In another option, the lead has one or more weak points for cutting the lead after the biasing assembly is in the strained state and the lead continues to be pulled.

In examples, the biasing assembly may comprise a resiliently deformable member according to the first aspect. The resiliently deformable member may thus, in particular examples, have two ends each configured to pull a respective part of the device thereby biasing the device in the expanded configuration, at least one end of the resiliently deformable member being free in the relaxed state, the resiliently deformable member being configured for being stretched and for each free end to be attached to its respective part of the device while the device is in the closed configuration, thereby transferring the biasing assembly from the relaxed state into the strained state. Optionally, in the relaxed state, the resiliently deformable member may be configured to be stretched by pulling each free end via a respective lead. The lead may be any type of lead according to the second aspect as earlier-defined and/or any type of lead according to the first aspect as earlier-defined. Further optionally, at least one free end of the resiliently deformable member configured to be pulled via a respective lead may comprise a tunnel for threading the lead therein. Additionally or alternatively, the respective lead may have one or more weak points for cutting the lead after the free end is attached to its respective coupling head and the lead continues to be pulled. Additionally or alternatively, at least one free end of the resiliently deformable member may comprise an extension configured for protruding out of its respective part of the device after said end is attached to its respective coupling head. In such a case, the resiliently deformable member may comprise a cutting zone at the basis of the extension for cutting the extension after said end is attached to its respective coupling head and the lead continues to be pulled.

In examples where the device comprises no locking assembly, the device may be configured to transfer from the expanded configuration into the emptying configuration upon the biasing assembly (e.g., resiliently deformable member) stopping to force the coupling heads together.

Optional features of the device according to any of the first and second aspects are now discussed.

In examples, the device may further comprise a locking element, such as a container (for at least partially enclosing, the device in the closed configuration), arranged to temporarily maintain the device in the closed configuration and configured to release the device only at some point after dosing. The container may optionally be a capsule (i.e., present a capsule shape).

The locking element or container may optionally be configured to degrade in environment conditions of the predetermined location of the GI tract. For example, when the predetermined location is the stomach, the locking element or container may be configured to degrade in stomach environmental conditions. As another example, when the predetermined location is the ICV, the locking element or container may be configured to degrade in small intestine environmental conditions (e.g., same conditions as the ICV), and resistant at standard stomach environmental conditions. Such a locking element or container facilitates administration.

The locking element may optionally be configured to release the device in the expanded configuration when or before reaching the predetermined location (e.g., stomach or small intestine). For example, when the predetermined location is the ICV, the locking element may be configured to release the device in the expanded configuration when the device is positioned in the small intestine. The device may in such a case be configured to still reach the ICV by motility. In examples where the predetermined location is the stomach, the locking element or container may be at least partially made of a material degrading at standard stomach environmental conditions. In examples where the predetermined location is the within the intestine such as the ICV, the locking element or container may be at least partially made of a material degrading at standard small intestine environmental conditions and resistant at standard stomach environmental conditions. For instance, the locking element or container may be at least partially coated with or made of an enteric polymer.

The device may comprise a container additionally to a locking element, and the device may be contained in the container in the closed configuration. In such examples, the locking element may optionally be different in shape from a container, and/or the container may optionally be non-locking (i.e., present no locking capability, thus all left to the locking element).

In examples of the device according to the first aspect, and in examples of the device according to the second aspect which comprise a resiliently deformable member as earlier-discussed, the resiliently deformable member may be made of an elastic material. For example, the elastic material may be silicone, such as between shore A 40 and shore A 80. Silicone is of a relatively low impact to the subject, even in case of frequent exposure due to a dosage of the device between once a week and once or several times a day. Yet, due to the locking assembly and/or the relaxed state during storage, a resiliently deformable member in silicone presenting sufficient expansion and/or retention force may be achieved while using relatively little material and thus keeping size relatively small. For instance, the silicone amount used in the resiliently deformable member may be less than 300 mg, for example less than 150 mg.

Alternatively, the resiliently deformable member may be made of metal, such as stainless steel or Nickel titanium. Mechanical properties of metal combined with the locking assembly and/or the relaxed state during storage allow achieving a resiliently deformable member presenting sufficient expansion and/or retention force while using particularly little volume of material and thus keeping size particularly small. For instance, the metal amount in the resiliently deformable member may be less than 500 mg, for example less than 250 mg. At such quantities, ingestion even on a frequent basis induces relatively little impact.

Additionally or alternatively in such examples, the resiliently deformable member may be in the form of a tube. Additionally or alternatively, the resiliently deformable member may be a spring, for example a spiral (or helical) spring.

In examples, the device may have a shelf-life for example in the folded configuration, of longer, than three months, for example longer than four months, or for example longer than six months, one year, two years, or five years. In other words, the device may be stored for as long after manufacturing, and still be functional, such as by still presenting a sufficient unfolding force and/or retention force. In particular examples, the device may present no “straining before dosing” capability, and yet have such a shelf-life duration, e.g., a shelf-life two years. Without wishing to be bound to theory, the unfolding mechanism exerts minimal strain on the resiliently deformable member, e.g., the member is minimally stretched compared to its maximal possible stretching resulting in minimal plastic deformation of the member while the device is in folded configuration, thereby enabling a long shelf life in the folded configuration while in the unfolded (expanded) configuration enabling a structural resistance to pressures of 0.5 kg/cm2, or 1 kg/cm2 or up to 1.5 kg/cm2.

Additionally or alternatively, the device may present (e.g., on the day of manufacturing, at any time before one week after manufacturing, and/or at any time before two months after manufacturing or during the shelf life of the device where the shelf life is at least two years or at least three years) an unfolding force below 2.45 N (i.e., force required to prevent the expanded configuration of the device by the biasing assembly or resiliently deformable member) and/or a retention force above 3.92 N, more preferably more than 4.90 N (i.e., force required to transfer the device form the expanded configuration into a compact configuration such that it may move past the predetermined location of the subject, e.g., by being small enough to go through the ICV or the pyloric valve, e.g., even during a fasted state of the subject and the pyloric valve is thus enlarged). The locking assembly allows achieving an unfolding force different and smaller than the retention force, and for example an unfolding force below 2.45 N or about 1.47 N and a retention force (holding force) above 4.90 N, for example up to about 14.70 N. In particular examples, the unfolding force may be about 1.47 N or below 1.47 N, for example about 0.98 N. This achieves a particularly low risk of unfolding in the esophagus, while allowing the use of particularly little material (e.g., silicone for the resiliently deformable member). In particular examples, the holding force (retention) may be about 4.90 N or 9.80 N or about 14.70 N. In some embodiments, the ratio between the holding force and folding force is about 10:1, for example about 14.70 N/1.47 N.

A “fasted state” refers to the state of the gastric environment of a subject. In particular a fasted state refers to a state in which the last food intake (i.e., a meal/snack) is digested and emptied from the stomach. This would be at least three to four hours after a meal. In general, there is high probability of objects emptying from the stomach during the fasted state, emptying induced by pyloric valve/sphincter expansion to maximal size and emptying forces applied during migration motor complex phase III (which induces evacuation). For example, the Given Imaging capsule (>25 mm (millimeter) in length >11 mm in diameter), when given in a fasted state achieves an average gastric retention time of about 1-2 hr.

For example, the unfolding force, when tested according to the tests described with reference to FIG. 10A with a D2 value of 13 mm, may be between 1.47 and 4.90 N at t=0 and following storage of one week in a container in the folded/closed configuration. Additionally or alternatively, the unfolding force, when tested according to the tests described with reference to FIG. 10A with a D2 value of 13 mm may be less than 1.47 N. Yet additionally or alternatively, the retention force, when tested according to the tests described with reference to FIG. 10A with a D2 value of 16 mm may be higher than 2.45 N, more preferably higher than 4.90 N at t=0 and following one week of storage in a container in the folded/closed configuration.

In examples, the device in the closed configuration may have a compact shape. Such a compact shape facilitates frequent administration of the device, e.g., in particular by ingestion, such as daily ingestion.

The dimensions of the device in the closed configuration may optionally be such that it can be ingested and preferably fitted (e.g., with its optional container) in a cylinder of length of about 35 mm or less and/or of diameter of about 12 mm or less, preferably in a cylinder of length equal to or less than 32 mm or 30 mm, and/or of diameter equal to or less than 11 mm, more preferably a cylinder of length equal to or less than 30 mm and/or of diameter equal to or less than 10 mm or 9 mm, even more preferably a cylinder of length equal to or less than 29 mm and or of diameter equal to or less than 9 mm, even more preferably in a cylinder of length equal to 28 mm and of a diameter equal to 8.5 mm or more preferably in a cylinder of length equal to 27 mm and of a diameter equal to 8.4 mm. By “fitted in a cylinder of a certain size”, it is meant that a theoretical cylinder of that certain size may be imagined that would contain in its volume substantially all the material of the device in the closed configuration (wherein material falling on the boundary surface of the cylinder is said to be contained in the cylinder).

Additionally or alternatively, the device may present in the closed configuration a length higher than 20 mm and/or a diameter higher than 7 mm, preferably a length higher than 25 mm and/or a diameter higher than 8 mm, for example a length of about 27 mm and a diameter of about 8.4 mm The “length” designates the largest dimension of the device along a longitudinal direction. The “diameter” designates the largest dimension of the device in a plane orthogonal to the longitudinal direction. The device may optionally present an elongated shape such as a capsule shape, e.g., a shape of cylinder with rounded extremities, and the length and diameter may be those of such a shape. Such minimal dimensions of the device allow to achieve a sufficient expansion.

In specific implementations, the device may in the closed configuration be able to be fitted in a cylinder presenting a length of about 30 mm and/or a diameter of about 10 mm. Additionally or alternatively, the device may present in the closed configuration a length higher than 25 mm and/or a diameter higher than 8 mm. Thus, the length of the device may be between 25 mm and 30 mm and the diameter of the device may be between 8 mm and 10 mm. The device may be presented in the closed configuration in the form of a capsule (i.e., the device comprises a capsule container as earlier-discussed) of size between “000” and “00” in its diameter (between about 9.55 mm and 8.18 mm) and a length of about 25 to 30 mm. More preferably the device presents in the closed configuration the form of a capsule of having a diameter of a capsule between “000” and “00”. For example, the device may fit into a capsule shell of standard sizes 000 to 00. Alternatively, the device may be presented in the closed configuration in the form of a capsule of size “0”, having a diameter of about 7.34 mm and a length of about 18.44 mm.

In examples, the dimensions of the device in the expanded configuration may be such that it can be fitted in a sphere of a diameter of about 35 mm or less, and preferably of about 30 mm or 25 mm or less. In the case where the predetermined location is within the intestine such as the ICV, this allows the device to transit through the small intestine of the subject even if the device is in the expanded configuration.

In examples, a volume occupied by the device in the expanded configuration may be larger than a sphere of a diameter of about 17 mm, and optionally larger than a sphere of a diameter of about 20 mm. In other words, an external hull of the device in the expanded configuration sticks out of a sphere of a diameter of about 17 mm and optionally of a sphere of a diameter of about 20 mm or 22 mm. This allows retention, for example by the pyloric valve (even in a fasted state) or by the ICV.

In examples, in the expanded configuration, any one or more of a shape of the device and/or a size of the device and/or a structural rigidity of the device may prevent passage of the device through the ileocecal valve or the pyloric valve (e.g., even during a fasted state of the subject and the pyloric valve is thus enlarged), when the device is positioned at the ileocecal valve.

In examples, the device may have in the expanded configuration a convex hull presenting a sphericity above 0.8, or about 0.85 to about 1 and/or a ratio between a maximal (e.g., planar) circumference and a minimal (e.g., planar) circumference below 1.5 or about 1 to about 1.2. Sphericity {\diSplayStyle \Psi} can be calculated based on the Wadell definition of sphericity, i.e., the ratio of the surface area of a sphere with the same volume as the given particle to the surface area of the particle. This may limit risks of damage to body tissue in any orientation. In examples, the device may maintain a spherical structure, (e.g., as in the examples of the device in the figures), and/or have an expanded configuration with a maximal diameter of less than 25 mm. This lowers the risk of pressure on stomach antral tissue or intestinal tissue, in any orientation.

In examples, the device may further comprise a trigger assembly configured to cause the device to transfer from the expanded configuration into the emptying configuration upon activation. In examples, the biasing assembly (e.g., resiliently deformable member) may form an opening assembly configured to transfer the device from the closed configuration into the expanded configuration, and the trigger assembly and the opening assembly may form a single transferring assembly configured to transfer the device from the closed configuration into the expanded configuration and to transfer the device from the expanded configuration into the emptying configuration upon activation.

Optionally, the trigger assembly may be configured such that the device in the expanded configuration maintains structural integrity (i.e., any one or more of shape, size and/or structural rigidity) until transfer in the emptying configuration is effected (i.e., achieved).

Additionally or alternatively, the trigger assembly may be configured to activate when the device in the expanded configuration is exposed to at least one activation signal.

For example, the at least one activation signal may comprise exposure of the device in the expanded configuration to standard environmental conditions of the predetermined location for a predetermined residence time period, wherein preferably the predetermined residence time period is 12 hr, one day, two days, three days or more and/or twelve weeks or less, for example one month, one week, two weeks, three weeks, 12 hr, one day, two days or three days (i.e., the expanded device is designed to be retained the predetermined location for said residence time period before being transferred into the emptying configuration).

Additionally or alternatively, the at least one activation signal may comprise exposure of the device in the expanded configuration to at least one activation environmental condition. In examples where the predetermined location is a location within the small intestine or the large intestine, for example the ICV, the at least one activation environmental condition may comprise a surrounding environment reaching a predetermined pH threshold, for example reaching below pH 5. In examples where the predetermined location is the stomach, the at least one activation environmental condition may comprise a surrounding environment reaching a predetermined pH threshold, for example reaching above pH 6.

Yet additionally or alternatively, the at least one activation signal may comprise an to electromagnetic or magnetic or ultrasound signal.

In examples, the trigger assembly may comprise one or more support elements configured to temporarily maintain the device in the expanded configuration. For example, one or more support elements may be configured to be disabled upon activation of the trigger assembly thereby causing the device to transfer into the emptying configuration. In particular, when the device in the expanded configuration is exposed to at least one activation signal, one or more respective support elements may be configured to be disabled. Optionally, when the device in the expanded configuration is exposed to at least one activation signal, one or more respective support elements may be configured to be disabled by degradation.

In examples of the device according to the first aspect, one or more support elements may be configured to temporarily secure the resiliently deformable member to the at least one respective coupling head, and thereby temporarily maintain the device in the expanded configuration. In examples of the device according to the second aspect comprising a resiliently deformable member, one or more support elements may be configured to temporarily secure the resiliently deformable member to respective parts of the device, for example each respective part pulled by the resiliently deformable member, and thereby temporarily maintain the device in the expanded configuration. Optionally, one or more support elements may be configured to cooperate with the biasing assembly (e.g., resiliently deformable member) so that, when the one or more support elements are disabled, the biasing of the biasing assembly is prevented and the device thereby transfers into the emptying configuration.

Additionally or alternatively, one or more support may be configured to be disabled when a surrounding pH reaches a predetermined pH threshold. Such one or more support elements may optionally be at least partially made of a pH dependent polymer configured to dissolve when the surrounding pH reaches the predetermined pH threshold. Additionally or alternatively, one or more support elements may be configured to be disabled when a surrounding pH reaches a predetermined pH threshold. Additionally or alternatively, one or more support elements may comprise a material configured to degrade at standard environmental conditions of the predetermined location, for example when the surrounding pH is that of the predetermined location.

In examples where the predetermined location is within the intestinal tract such as at the ICV, one or more support elements may be at least partially made of a material configured to degrade at standard ICV environmental conditions. For example, one or more support elements comprise a material configured to degrade when the surrounding pH is between 6.5 and 7.5. Additionally or alternatively, one or more (other) support elements may be at least partially made of a pH dependent polymer configured to dissolve when the surrounding pH reaches a predetermined pH threshold, preferably when the surrounding pH reaches below pH 5.

In examples where the predetermined location is the stomach, one or more support elements may be at least partially made of a material configured to degrade at standard stomach environmental conditions. For example, the one or more support elements may comprise a material configured to degrade when the surrounding pH is between 1 and 3 or between 1 and 4 or 5. Additionally or alternatively, one or more (other) support elements may be at least partially made of a pH dependent polymer configured to dissolve when the surrounding pH reaches the predetermined pH threshold, preferably when the surrounding pH reaches above pH 6. In such cases, at least one of the support elements may be activated by dosing a tablet that alters the stomach pH so to disassemble and empty the device from the stomach (by transferring the device from the expanded configuration into the emptying configuration). Such treatments may be considered as a “safety mechanism”, e.g., in case emptying from stomach is required. Furthermore, the device disassembles to small parts, enabling easy and safe emptying from the body, for example through the GI tract. In some embodiments, each part has dimensions of about 20 mm×5 mm×3 mm or less.

In examples, the one or more support elements may comprise several support elements each configured to be disabled upon the device in the expanded configuration being exposed to a different activation signal, thus causing the device to transfer into the emptying configuration. This provides different emptying patterns.

For example, the device may comprise one or more first support elements of the type degrading when the surrounding pH reaches a threshold (corresponding to a non-standard environment condition), and/or one or more (distinct) second support elements degrading at standard environment conditions of the predetermined location. Optionally, the one or more first support elements may be configured to degrade faster than the one or more second support elements. The one or more second support elements may be configured to degrade naturally after a predetermined residence time at the predetermined location due to standard environmental conditions, and the one or more first support elements may be configured to degrade on decision, by actively changing such environmental conditions, for example into a non-standard pH configured for degrading the first support elements.

In examples, at least one (e.g., each) of the one or more support elements may be an integrally formed component presenting a diameter (i.e., length of longest straight segment from a point of object to another point of object) shorter than a half (preferably shorter than a fourth) of a diameter of the device in the expanded configuration. In examples where the device is configured to transfer from the expanded configuration into the emptying configuration by disassembling into at least two disassembled subcomponents, such support element may present a diameter shorter than a half (preferably shorter than a fourth) of a diameter of each disassembled subcomponent (e.g., at the time when the disassembling occurs in case the disassembled subcomponent is not rigid). Such a support element may enable localized degradation until emptying of the device, thus little affecting (at least not substantially) structural rigidity, shape, and/or size of the device while in the expanded configuration. The integrally formed component may be separate or distinct from at least part (e.g., all) of an optional carried/embedded load of API.

In examples, the one or more support elements may be configured to form one or more structural weak points of the device to enable a collapse of the device into the emptying configuration after the device is exposed to the activation signal. The one or more structural weak points may enable (preferably sudden) disassembly of the device into at least two disassembled subcomponents. This may contribute to improving a (preferably rapid) transfer from the expanded configuration into the emptying configuration. This also participates in enabling the device to maintain structural integrity until the device transfers into the emptying configuration thereby improving device efficiency until collapse. Furthermore, the components of the device which play a significant role in the main functionalities of the device such as retention, and/or optional API release, may generally be configured to not be affected by the activation signal. For example, these components (e.g., arms and/or biasing assembly, e.g., resiliently deformable member) may not be made of a material configured to degrade at (i.e., may be made of a material configured to resist to) the environmental conditions of the predetermined location in embodiments in which the activation signal is exposure to such standard environmental conditions for a predetermined residence time period.

In examples, one or more support respective elements may be configured to be disabled in less than 7 days, 3 days, 1 day or ½ day after the device is exposed to a respective activation signal (preferably said respective activation signal comprising the exposure to standard environmental conditions of the predetermined location).

In examples, the device may comprise a meshed structure having one or more openings configured for allowing chyme flow through the device, when the device is in the expanded configuration.

In examples, the device may be configured for release of at least one active pharmaceutical ingredient (API) at the predetermined location of the GI tract.

For example, the device may carry (i.e., embed) a load of at least one API and be configured, when the device is in the expanded configuration and positioned at the predetermined location of the GI tract, for releasing at least partially the API. In particular, when the predetermined location is the stomach, the device may carry such load for release in the stomach. When the predetermined location is within the intestine (such as the ICV), the device may optionally carry such a load for release and/or optionally have a trapping configuration for release from a trapped object). The API may be for treatment of an illness, and/or may be dosed at a frequency between once a week and once or more a day (i.e., daily administration). Optionally, the device may be configured for allowing chyme flow therethrough in the expanded configuration, as this facilitates API release.

Additionally or alternatively, in the particular case where the predetermined location of the GI tract is within the intestine, for example the ileocecal valve, the device being configured in the expanded configuration to be retained at the ileocecal valve of the subject while allowing chyme flow, and to pass through the ileocecal valve in the emptying configuration, the device may in examples be configured for blocking a cooperating ingestible object while allowing chyme flow, when the device is positioned at the ileocecal valve and in the expanded configuration. Thus, the expanded configuration forms a trapping configuration. For instance, the device may comprise a trapping assembly configured for blocking such a cooperating ingestible object while allowing chyme flow, when the device is positioned at the ileocecal valve and in the expanded configuration.

In examples, the cooperating ingestible object may be an oral dosage form comprising a quantity of at least one API. Thus, the device may be configured for releasing a carried load of API and/or a quantity of API of a (trapped) cooperating ingestible object.

In examples of the trapping functionality, the device or trapping assembly may comprise a meshed structure having one or more openings configured for preventing the cooperating object from passing therethrough (e.g., and for allowing chyme flow through the device), when the device is in the expanded or trapping configuration. Additionally or alternatively, the ingestible cooperating object may be an oral dosage form having predetermined minimal external dimensions. Additionally or alternatively, the device or the trapping assembly may be configured for blocking rigid spherical objects having a diameter above a trapping threshold diameter, wherein preferably the trapping threshold diameter is in the range of about 7 to 12 mm, such as about 9 mm. Additionally or alternatively, the device may further comprise an external padding structure. Such an external padding structure may be as disclosed in international patent application No. PCT/IL2020/050941 filed on 30 Aug. 2020, which is incorporated herein by reference.

Options for carrying the load of API are now discussed.

The device may comprise an inner space in the closed configuration formed between components of the device, the inner space containing at least part of the load of the API. For example, the device may comprise at least one portion or component which comprises an exposed cavity, the exposed cavity containing at least part of the load of the API. The at least one portion or component may optionally comprise a peripheral wall, the peripheral wall having apertures formed thereon, the apertures providing exposure of the exposed cavity. In various embodiments of the devices disclosed herein, there may be present 1, 2, 3, 4, 5, 6, 7, 8, or more cavities capable of harboring a load of API or APIs (i.e. one or more dosage form(s) each independently comprising API(s)). Additionally or alternatively, the device may comprise an exposed recess, the exposed recess lodging at least part of the load of the API. Such an exposed recess for carrying at least part of the load of the API may be as disclosed in international patent application No. PCT/IL2020/050941 filed on 30 Aug. 2020, which is incorporated herein by reference. Additionally or alternatively, the device may comprise a coating on an exposed surface, the coating containing at least part of the load of the API. Such a coating for carrying at least part of the load of the API may be as disclosed in international patent application No. PCT/IL2020/050941 filed on 30 Aug. 2020, which is incorporated herein by reference. Additionally or alternatively, the load of the API may be carried in any type of dosage form for example solid, semi-solid, powder, gel, and/or liquid form.

Additionally or alternatively, the load of the API may be contained in an embedded dosage form which occupies at least 5% of the volume of a convex hull of the device in the closed configuration, preferably at least 10%, at least 15%, or at least 25% of the volume of a convex hull of the device in the closed configuration.

In examples, the device the device is configured for delaying release of the API, for example such that less than 20% or less than 10% of the API is released within 0-2 hr post dosing. Such a delay mechanism allows to configure release of the API such that it occurs only at a certain point of time and/or at a certain location of the GI tract, for example exactly when or slightly before reaching the ICV when the predetermined location is the ICV and the device travels along the intestine and potentially unfolds and/or has its optional container degraded well-before reaching the ICV. Yet, the delay mechanism may be such that the device releases the API throughout retention time. Yet, the delay mechanism may be such that the device releases >50%, more preferably >75% of the API at about 2 hr before awakening. Such a delay mechanism also allows take the device in combination with an immediate release oral dosage form, and have the immediate release dosage form release API first, and then only after a certain point of time have the device release the carried API. The APIs of the device and the immediate release dosage form may be the same or different.

In examples, the device further comprises an external envelope. The external envelope of the device in the expanded configuration may be configured to contact the subject tissue and the external envelope of the device may be configured to avoid damaging the subject tissue. For example, the external envelope of the device may be flexible and/or blunt to avoid damaging the subject tissue. In particular when the predetermined location is within the intestine such as the ICV, the device in the expanded configuration may be configured to be capable of transiting through the small intestine to the predetermined location (e.g., ICV) by standard GI motility without damaging the small intestine. In particular when the predetermined location is the stomach, the device in the expanded configuration may be configured to be capable of moving in the stomach by standard stomach motility without damaging the stomach.

In examples, the device may be configured to transfer from the expanded configuration into the emptying configuration by changing shape. Additionally or alternatively, the device may be configured to transfer from the expanded configuration into the emptying configuration by changing size. Additionally or alternatively, the device may be configured to transfer from the expanded configuration into the emptying configuration by decrease of structural rigidity. Additionally or alternatively, the device may be configured to transfer from the expanded configuration into the emptying configuration by disassembling into at least two disassembled subcomponents.

In examples, the device may be such that in the closed configuration, the device can pass through the ileocecal valve of the subject when positioned at the ileocecal valve. Additionally or alternatively, the device in the closed configuration or emptying configuration may be able to pass through the pyloric valve of the subject when positioned in the stomach, in examples even if the subject is not fasted and the pyloric valve is thus reduced in size. Additionally or alternatively, an external envelope of the device in the emptying configuration and an external envelope of the device in the closed configuration may be of same dimensions. Additionally or alternatively, the closed configuration of the device may be the same as the emptying configuration of the device.

In examples, the device may further comprise a labelling element enabling detection of the device in a subject by external imaging means, wherein the imaging means is optionally X-ray imaging.

In examples, the device may further embed a camera and/or one or more measurement devices (such as one or more sensors for local measurement of pH and/or motility).

Optional features relative to structure of the first aspect are now discussed.

In examples, each coupling head may comprise one or more rigid and/or integrally formed components, each coupling head preferably being rigid and integrally formed.

In examples, the resiliently deformable member may be arranged between the arms. Additionally or alternatively, each arm may be arranged longitudinally alongside each other. For example, the device may comprise at least three or at least four flexible arms, the resiliently deformable member being optionally arranged between the at least three or at least four arms. Each arm may be part of an optional trapping assembly.

Preferably, the device comprises at least three arms, even more preferably four arms. This imparts a 3D shape to the device in the expanded configuration, thus achieving high retention capability.

In examples, the device may further comprise one or more circumferential belts circling around the flexible arms, and/or one or more circumferential threads circumferentially linking the flexible arms. In all cases such circumferential thread(s) and/or belt(s) may increase structural rigidity and durability of the device and strengthen its retention capability. In addition, these may be part of an optional trapping assembly, for example in case the predetermined location is within the intestinal tract (e.g., ICV). Such circumferential belt(s) and/or circumferential thread(s) may be as disclosed in international patent application No. PCT/IL2020/050941 filed on 30 Aug. 2020, which is incorporated herein by reference.

In examples, the resiliently deformable member may be releasably secured to at least one of the first and second coupling heads, at least in the expanded configuration and in the optional dosing sub-configuration. For example, the resiliently deformable member may be configured to be released from the at least one of the first and second coupling heads when the device in the expanded configuration is exposed to a predetermined activation signal. The first end of each flexible arm may be coupled to the first coupling head releasably, and/or the second end of each flexible arm may be coupled to the second coupling head releasably. In examples, in the expanded configuration, said first and second ends may be each inserted in a respective cavity of the first and second coupling heads, the resiliently deformable member maintaining said first and second end each secured inside the respective cavity. Said first and second ends may each be rotatable in the respective cavity. Addition or alternatively, when the device transfers from the expanded configuration into the emptying configuration, said first and second ends may be each dimensioned to move out of the respective cavity (i.e., the dimensioning enables such moving out), the device being thereby configured for the disassembling of the flexible arms from the first and second coupling heads. In particular, the detachment of the arms from the head may be possible when the device is in the emptying configuration, and still the detachment of the arms from the head cavities may be induced by GI motility. For example, the transfer into the emptying configuration may move the arm ends out of abutment with abutment surfaces in the cavities, thereby enabling the ends to move out of the cavities.

In examples, the resiliently deformable member may be releasably secured to at least one of the first and second coupling heads, and the resiliently deformable member may be configured to be released from the at least one of the first and second coupling heads when the device in the expanded configuration is exposed to a predetermined activation signal, thereby causing the device to transfer from the expanded configuration into the emptying configuration. Such an activation signal may be as earlier-described, for instance disabling (e.g., degrading) a trigger assembly (e.g., with one or more support elements).

In examples, the device further comprises a support tube, the resiliently deformable member being arranged inside the support tube. The device may carry at least part of a load of API inside or on the support tube, for example in an interstice formed between the support tube and the resiliently deformable member. Additionally, in examples of the device according to the first aspect, the locking member may be arranged inside the support tube. Optionally, the at least part of the load of the API may be carried inside or on the support tube in a solid, semi-solid, powder, gel, and/or liquid form. Further optionally, the support tube may have apertures formed thereon, the apertures providing exposure to the inside of the support tube. The support tube may in particular have one or more peripheral grooves each lodging a ring-shaped form containing the API. Such a support tube and its optional capability of carrying at least part of the load of the API may be as disclosed in international patent application No. PCT/IL2020/050941 filed on 30 Aug. 2020, which is incorporated herein by reference.

In examples, at least one (e.g., each) arm is articulated and/or composed of a set of arm sections, wherein said arm sections (e.g., of each arm) are preferably rigid. For instance, at least one (e.g., each) arm section may comprise one or more integrally formed components. Optionally, the one or more integrally formed components of said at least one arm section may comprise one or more integrally formed components made of a rigid material, one or more integrally formed components made of a semi-rigid material, and/or one or more integrally formed components made of a flexible material. Additionally or alternatively, each integrally formed component of said at least one arm section is 3D printed or injection molded. In examples, at least two (e.g., all) arm sections of at least one (e.g., each) flexible arm are coupled end to end with a pivot-type coupling. For example, the pivot-type coupling may comprise transversal hinge holes of the arm sections of the at least two arm sections and a hinge connector passing through the transversal hinge holes.

In examples, each flexible arm may consist of two arm sections, each arm section being coupled at one end to the first or second coupling head and at the other end to the other arm section. Optionally, each arm section is substantially of a same length, the device having a generally bipyramidal shape in the expanded configuration, preferably a generally octahedral shape.

In examples, at least one arm section comprises an exposed cavity.

For example, the at least one arm section may comprise two arm section components attached one to another and forming the exposed cavity there between. For instance, the two arm section components may be snapped one to another.

Additionally or alternatively, the at least one arm section may comprise a peripheral wall, the peripheral wall having apertures formed thereon, the apertures providing exposure of the exposed cavity. Additionally or alternatively, the exposed cavity may contain at least part of a load of API. Optionally, the at least part of the load of the API may be contained in the exposed cavity in a solid, semi-solid, powder, gel, and/or liquid form.

In examples alternative to rigid material or semi-rigid material, each flexible arm may incorporate or be (e.g., partially) made of an elastic material, the resiliently deformable member being configured to bend the arms in a rounded or semi rounded shape. For example, each flexible arm may comprise an integrally formed component made of the elastic material, the resiliently deformable member being configured to bend the integrally formed component in a rounded shape. Additionally or alternatively, the elastic material is silicone. Such flexible arms made of an elastic material may be as disclosed in international patent application No. PCT/IL2020/050941 filed on 30 Aug. 2020, which is incorporated herein by reference.

In examples, the arms may be at least partially made of a material that gradually erodes in the intestine, so as to facilitate emptying (e.g., after disassembly of the device). This, however, does not interfere with the capability of retention until emptying (e.g., disassembly) occurs. In examples, there is no gradual decrease in the size or retention capability of the device during gradual erosion of the arm while the device is at the predetermined location.

In examples where the predetermined location is within the intestine (e.g., the ICV), the arms may be coated with a material resistant at standard stomach environmental conditions and/or enabling the device to reach the ICV region substantially intact.

According to a third aspect, an assembly is provided comprising a packaging and, inside the packaging, at least one (e.g., a plurality) of any example of the device according to any of the first and second aspects or any example thereof.

The packaging may comprise one or more storage locations such as blisters (e.g., arranged in a grid), thus forming a blister pack. Each storage location or blister may comprise a respective device. The devices may comprise a container, for example a capsule container. The devices may each comprise each one or more respective leads protruding out of the container. The one or more leads may be attached to the packaging, such that when pulling the device from its location on the packaging, e.g., out of a torn blister, the one or more leads may strain the device, i.e., transfer it into the dosing sub-configuration, as earlier-described.

According to a fourth aspect, an oral dosage form is provided for administering to a subject, the oral dosage form being intended for use in cooperation with any example of the device according to any of the first and second aspects which comprises a triggering assembly, when said device is positioned at the predetermined location of the GI tract of the subject in the expanded configuration, wherein the dosage form comprises an effective amount of an emptying agent, wherein the amount of the emptying agent is sufficient to cause at least one activation environmental condition to be reached at the predetermined location, thereby causing the device to transfer from the expanded configuration into the emptying configuration.

For example, the device may comprise one or more support elements forming structural weak points and configured to be disabled by the at least one activation environmental condition, thereby causing the device to transfer into the emptying configuration.

Optional features of the oral dosage according to the fourth aspect where the predetermined location is the ICV are now discussed.

In examples, the oral dosage form may be a tablet having an oval or elliptical or capsule shape having a length of about 10 mm to 30 mm and a diameter of about 7 mm to 12 mm.

In examples, the amount of the emptying agent may be sufficient to cause the ileocecal valve environment to reach at least one activation environmental condition thereby causing the device to transfer into the emptying configuration.

In examples, release of a substantial amount of the emptying agent may downsize the dosage form so that it is capable of passing through the optional trapping assembly of the device.

In examples, the emptying agent may comprise an amount of an acid sufficient to bring an ileocecal valve environmental pH below a pH threshold, for example pH 5. For example the acid may comprise short chain organic acid such as citric acid or tartaric acid. The coating may comprise an enteric polymer or time dependent eroded polymer.

Optional features of the oral dosage according to the fourth aspect where the predetermined location is the stomach are now discussed.

In examples, the agent may be sufficient to cause the stomach environment to reach at least one activation environmental condition thereby causing the device to transfer into the emptying configuration.

In examples, the emptying agent may comprise an amount of a base (antacid) sufficient to bring a stomach environmental pH above a pH threshold, for example pH 5.5, 6.0, 6.5, or 7. For example, the base may comprise aluminum hydroxide, magnesium carbonate, calcium carbonate sodium bicarbonate or such.

According to a fifth aspect, an oral dosage form is provided for administering to a subject suffering from a condition which may benefit from local dispensing at the ileocecal valve region an API, the oral dosage form being intended for use in cooperation with any example of the device according to any of the first and second aspects where the expanded configuration is a trapping configuration and/or which comprises a trapping assembly, and which is configured for the predetermined location to be the ICV, when said device is positioned at the ileocecal valve of the subject in the expanded configuration, wherein the dosage form comprises:
(a) an amount of an API effective to treat said condition;
(b) a coating for inhibiting release of said API in the gastric environment and enabling release of said API in the ileocecal valve region;
(c) external dimensions for enabling the dosage form to be blocked by said device when reaching the ileocecal valve region.

Optional features of the oral dosage according to the fifth aspect are now discussed.

In examples, the coating may be such that less than 5% of the API is released in the stomach, optionally less than 2%.

In examples, release of a substantial amount of the API may downsize the dosage form so that it is capable of passing through the trapping assembly of the device.

In examples, the dosage form may comprise a controlled release formulation wherein less than 50% of the API is released from the dosage form after exiting the stomach and before reaching the ileocecal valve.

In examples, the condition may be an inflammatory bowel disease (IBD), optionally ulcerative colitis (US) or Crohn's disease.

According to a sixth aspect, a method of treatment of a subject is provided, which comprises providing any example of the device according to any of the first and second aspects, which is configured for release of at least one API at the predetermined location of the GI tract (where the device is configured to be retained). Such a method of treatment is intended for a subject who suffers from a condition which benefits from local dispensing of the API at the predetermined location. Thus, by merely administering the device to the subject, the method enables treating the subject.

In a first example of the sixth aspect, the predetermined location is the stomach and the method is for treating a condition which benefits from local dispensing of an API in the stomach. In such a case, the device is any example according to any of the first and second aspects, which is configured in the expanded configuration to be retained in the stomach of the subject, and to pass through the pyloric valve in the emptying configuration. The device further carries a load of the API and is configured, when the device is in the expanded configuration and positioned in the stomach, for releasing at least partially the API. Thus, administration of the device treats the condition in said subject.

Optional features of the method according to the first example of the sixth aspect are now discussed:

In examples, the condition may be a gastric disease, such as ulcer or cancer.

In examples, the API released in the stomach may be absorbed in the small intestine so as to treat said condition or improve treatment of said condition.

In examples, the device may be administered to the subject under fasted conditions. In examples, the API is a drug that may have at least one of the following: slow and or incomplete intestinal absorption, short biological half-life and therapeutic index (e.g., narrow absorption window), and/or high degradation in intestine.

In examples, the method may comprise subsequently administering any oral dosage form according to the fourth aspect, thereby emptying the device.

In examples, the subsequently administering of the oral dosage form may be performed a predetermined time after administering the device, the device being retained in the stomach during the predetermined time. For example, the predetermined time may be at least 6 hours, at least 12 hours, at least 24 hours, at least 48 hours, at least three days, at least one week, at least two weeks, at least three weeks, or at least one month.

In examples, the method may comprise subsequently administering one or more subsequent devices according to need or at constant intervals.

In examples, the device may have in the expanded configuration a convex hull presenting a sphericity above 0.8, or about 0.85 to about 1 and/or a ratio between a maximal circumference and a minimal circumference below 1.5 or about 1 to about 1.2.

In a second example of the sixth aspect, the predetermined location is the ICV and the method is for treating a condition which benefits from local dispensing of an API at the ICV. In such a case, the device is example according to any of the first and second aspects, which is configured in the expanded configuration to be retained at the ICV of the subject, and to pass through the ICV in the emptying configuration. In this second example, the device further carries a load of the API and is configured, when the device is in the expanded configuration and positioned at the ICV, for releasing at least partially the API. Thus, administration of the device treats the condition in said subject.

In a third example of the sixth aspect, another method is provided for treating a condition which benefits from local dispensing of an API at the ICV of a subject suffering from said condition. The method comprises administering to said subject any example of the device according to any of the first and second aspects, which is configured for blocking a cooperating ingestible object while allowing chyme flow, when the device is positioned at the ICV and in the expanded configuration. For example, the device may comprise a trapping assembly configured for blocking such a cooperating ingestible object while allowing chyme flow, when the device is positioned at the ICV and in the expanded configuration. The method comprises subsequently administering any oral dosage form according to the fifth aspect and containing the API.

Optional features of the method according to the second and/or third example of the sixth aspect are now discussed:

In examples, the device may further carry a load of the API and be configured, when the device is in the expanded configuration and positioned at the ICV, for releasing at least partially the API, thereby further treating said condition in said subject (in addition to the trapped oral dosage form). Alternatively, the device may carry no such load.

In examples, the oral dosage form may be administered to the subject while the device is positioned at the ileocecal valve in the expanded configuration.

In examples, the oral dosage form may be administered to the subject under fasted conditions.

In examples, the oral dosage form may be administered to the subject at least 2 hours, at least 3 hours, at least 4 hours, at least 5 hours, at least 6 hours, at least 7 hours, at least 8 hours, at least 12 hours, at least 24 hours, or at least 48 hours after administration of the device.

In examples, the oral dosage form may be administered to the subject about 5 hours to 10 hours after administration of the device.

In examples, the dosage form is administered to the subject according to need.

In examples, one or more subsequent dosage forms may be administered to the subject at constant intervals. For example, the one or more subsequent dosage forms may be administered to the subject twice a day, every day, every 2 days, every 3 days, every 4 days, every 5 days, every 6 days, every 7 days, or at constant intervals of more than 7 days.

Optional features of the method according to the second and third examples of the sixth aspect are now discussed:

In examples, the condition may be Inflammatory Bowel Disease (IBD). For example, the IBD may be ulcerative colitis. As another example, the IBD may be Crohn's disease.

In examples, the treatment may alleviate at least one IBD symptom in the subject.

In examples, the at least one IBD symptom may be selected from weight loss, macroscopic colonic damage, colonic ulceration, intestinal and/or peritoneal adhesion, diarrhea, bowel wall thickening, nauseas, vomiting, abdominal cramps, abdominal pain, intestinal bleeding, intestinal inflammation, GI tract inflammation, rectal bleeding, tiredness, anemia, fistulae, perforations, obstruction of the bowel or a combination thereof.

In examples, the oral dosage form and/or carried API may comprise mesalamine or a pharmaceutically acceptable salt thereof.

In examples, the oral dosage form and/or carried API may comprise levodopa or a pharmaceutically acceptable salt thereof.

For example, the oral dosage form and/or carried API may comprise glatiramer acetate.

For example, the method may comprise subsequently administering an oral dosage form according to the fourth aspect, thereby emptying the device.

For example, the treatment may induce or maintain clinical remission in the subject. For example, method may comprise subsequently administering one or more subsequent devices according to need or at constant intervals.

According to a seventh aspect, a method of use is provided, which comprises providing any example of the device according to any of the first and second aspects and administering the device to a subject.

In examples, the method of use is for diagnosis and/or imaging of the subject. In such a case, the devices may be used in cooperation with an external imaging device, and/or embed a camera and acquire images therewith, and/or embed one or more measurement devices and acquire signals/measurements therewith (such as one or more sensors for local measurement of pH and/or motility).

According to a further aspect, a method of treatment of a subject is provided, which comprises providing any example of the device according to any of the disclosed aspects, which is configured for release of at least one API at the predetermined location of the GI tract (where the device is configured to be retained), where the device being configured to remain positioned at the predetermined location of the gastrointestinal tract for at least one week.

According to an eighth aspect, a kit is also provided which comprises any example of the device according to any of the first and second aspects, or any example of the assembly according to the third aspect, and/or one or more cooperating emptying oral dosage forms according to the fourth aspect. In examples wherein the predetermined location is the ICV region and the device is configured for blocking a cooperating ingestible object while allowing chyme flow, when the device is positioned at the ICV and in the expanded configuration, for example via a trapping assembly, the kit may optionally further comprise, additionally or alternatively to the one or more cooperating emptying oral dosage forms, one or more cooperating therapeutic oral dosage forms according to the fifth aspect. The kit may further comprise a container, for example a capsule shell configured to hold the device in a folded configuration. The kit may further comprise instructions for use.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1J show front views, perspective views, and exploded views of a device according to embodiments of the present disclosure.

FIG. 1K illustrates arm-head design including assembly and disassembly mechanism.

FIGS. 2A-2D show a cross section view of a trigger assembly in cooperation with a locking member and a resiliently deformable member according to embodiments of the present disclosure.

FIGS. 3A-3D show a cross section view of a biasing assembly in a closed configuration and an expanded configuration according to embodiments of the present disclosure.

FIGS. 4A-4F illustrate functioning of a trigger assembly when a device according to embodiments of the present disclosure is in an expanded configuration.

FIGS. 5A-5K show front views, perspective views, and exploded views of another example of a device according to embodiments of the present disclosure in an expanded configuration and in a closed configuration.

FIGS. 6A-6F show a further example of an assembly containing a device according to embodiments of the present disclosure.

FIGS. 7A-7E show a further example of an assembly containing a device according to embodiments of the present disclosure.

FIGS. 8A-8K show an example of an assembly containing a device according to embodiments of the present disclosure.

FIGS. 9A-9F show another example of an assembly containing a device according to embodiments of the present disclosure.

FIGS. 10A-10B show an example device and an example experimental assembly useful in a method of determining retention capability of a device in accordance with embodiments of the present disclosure.

FIG. 11 is a graph showing gastric retention of exemplary devices controlled by three different timer mechanisms compared to control tablets.

FIG. 12 is a graph showing gastric retention of exemplary devices controlled by three different timer mechanisms compared to control tablets in a single animal (beagle dog).

FIGS. 13A-13F are X-ray images of an exemplary device as viewed in regions of the GI tract of a beagle dog.

### DETAILED DESCRIPTION OF EMBODIMENTS

Described herein are some examples of devices and methods related to retentive devices for the gastrointestinal (GI) tract, and for example useful for local drug release at the ICV region or at the gastric region of a patient (human).

In the following detailed description, numerous specific details are set forth in order to provide a thorough understanding of the subject matter. However, it will be understood by those skilled in the art that some examples of the subject matter may be practiced without these specific details. In other instances, well-known methods, procedures and components have not been described in detail so as not to obscure the description.

As used herein, the phrase “for example”, “such as”, “for instance” and variants thereof describe non-limiting examples of the subject matter.

Reference in the specification to “one example”, “some examples”, “another example”, “other examples, “one instance”, “some instances”, “another instance”, “other instances”, “one case”, “some cases”, “another case”, “other cases” or variants thereof means that a particular described feature, structure or characteristic is included in at least one example of the subject matter, but the appearance of the same term does not necessarily refer to the same example.

It should be appreciated that certain features, structures and/or characteristics disclosed herein, which are, for clarity, described in the context of separate examples, may also be provided in combination in a single example. Conversely, various features, structures and/or characteristics disclosed herein, which are, for brevity, described in the context of a single example, may also be provided separately or in any suitable sub-combination.

As used herein, the terms “about” and “at or about” mean that the amount or value in question can be the value designated some other value approximately or about the same. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In examples, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

In the present application, the following terms and their derivatives may be understood in light of the below explanations:

As used herein, the terms “local dispensing” and “local release” and their derivatives may be used to refer to a regio-specific release/dispensing of a substance at a predefined location in the GI tract, for example in the stomach or at the ICV region.

The term “swallowable” may be used to refer to an object having dimensions enabling oral administration to a human subject. The term oral administration may refer to ingesting the object. A swallowable object may be characterized as an object being capable of being fitted in a cylinder, wherein a length of the cylinder is, for example, about 35 mm or less and a diameter of the cylinder is, for example, about 12 mm or less. In some embodiments, a swallowable object may be as small as 27 mm long and about 8.4 mm in diameter and able to fit into a standard capsule shell, for example a 00 size capsule shell.

As used herein, the term “predetermined location (of the gastrointestinal tract)” or equivalently “the location of the gastrointestinal tract”, or “the location” may be used to refer a particular location or a region along the GI tract. In examples, the predetermined location may be the “gastric region” and may be used to refer to a region substantially consisting of the stomach, and for example including the pyloric valve. In other examples, the predetermined location may be the “ileocecal region”, “ICV region” or “ileocecal valve region” and may be used to refer to a region substantially consisting of the lower part of the small intestine (terminal ileum), the ileocecal valve, the cecum and the ascending colon.

As used herein, the term “retained at the location of the gastrointestinal tract” means that the device, in the expanded configuration, and after reaching the (predetermined) location of the GI tract does not move past the location. The device may be configured, in the expanded configuration, to remain at the location of the gastrointestinal tract for one day, three days, one week, or longer.

For example, “retained in the stomach” means that the device, in the expanded configuration and after reaching the pyloric region, does not pass through the pyloric valve and rather stays in the stomach. In other words, the device remains positioned within the stomach though the device may make movements inside the stomach. In examples, the device may be prevented from passing through the pyloric valve even during a fasted state of the subject/patient, such as during night and/or sleep, that is, when the pyloric valve is thereby naturally enlarged. For example, this feature provides a significant advantage in that extended drug delivery, i.e., at least up to 8 hr or preferably up to 10 hr may continue in a fasted state (e.g., at night) and to enable sufficient plasma levels in the morning.

In another example, “retained at the ileocecal valve” means that the device, in the expanded configuration and after reaching the ICV region, does not pass through the ileocecal valve and rather stays in the ICV region directly before/proximal to the ileocecal valve. In other words, the device remains positioned within the lower part of the small intestine (terminal ileum), e.g., within 0-20 cm of the ICV. In examples, the device may remain fixed when retained that the ICV, or alternatively the device may make small movements such as back and forth movements (but keep being retained by the ICV and thus not enter the cecum).

As used herein “structural rigidity” may refer to the ability of an object to maintain its shape while being exposed to an external load.

The term “environmental conditions” may refer to biological, physical and/or chemical conditions of an environment i.e., a medium or milieu in which the device is positioned or intended to be positioned. The environmental conditions may include for example, temperature, pH, atmospheric pressure, gravity, electromagnetic field, vibration, glucose concentration, oxygen concentration, enzyme concentration, etc. In a predetermined part of the GI tract, for example the stomach or pyloric region, or the ICV region, standard environmental conditions may refer to average physiological conditions observed in said part of the GI tract. For example, standard pH conditions for different parts of the human GI tract are summarized in the table below:

| | pH along the GI tract (1) | | |
|---|---|---|---|
| | | 25th | 75th |
| GI segment | median | percentile | percentile |
| stomach | 1.5 | 1.1 | 1.8 |
| duodenum | 6.4 | 5.7 | 6.9 |
| proximal small intestine | 6.6 | 6.2 | 7.1 |
| mid small intestine | 7.1 | 6.6 | 7.4 |
| distal small intestine | 7.3 | 7.0 | 7.5 |
| (including ICV) | | | |
| cecum | 5.7 | 5.2 | 6.3 |
| ascending colon | 5.6 | 5.1 | 6.5 |
| transverse colon | 5.8 | 5.3 | 6.4 |
| descending colon | 6.6 | 6.3 | 7.0 |
| rectum | 6.6 | 5.8 | 7.1 |

As used herein “degradability” may refer to the ability of a device to lose structural rigidity under certain physiologic conditions. The degradation products may be excretable and/or absorbable by the body.

As used herein, the term “closed configuration” of a device may be the state of the device prior to administration where the device has a size that is suitable for swallowing or for oral manipulation with an endoscope or suitable for administration by colonoscopy (lower endoscopy). The closed configuration may also be referred to as a swallowing or swallowable configuration, collapsed configuration, compact configuration, compressed to configuration, deflated configuration, folded configuration or the like.

As used herein, the “expanded configuration” of the device may be the state of the device when it resides at the predetermined location. In some embodiments, the device may be orally administered, for example via ingestion or via upper/lower endoscopy, and the device may be delivered in the expanded configuration in at the predetermined location. The expanded configuration of the device notably prevents passage of the device, possibly transited by the GI motility, from the predetermined location, for example through the pyloric valve when the device is in the expanded configuration in the stomach (e.g., even if the subject is fasted and the pyloric valve thereby enlarged), or through the ileocecal valve when the device is in the expanded configuration in the small intestine. In these examples, the expanded configuration of the device may not block the pyloric valve or ileocecal valve and may enable chyme flow therethrough. The expanded configuration may also be referred to as opened configuration, inflated configuration, unfolded configuration, or the like.

In embodiments where the predetermined location is the ileocecal region, the device may be configured for trapping (i.e., blocking) a cooperating object, when the device is in the expanded configuration. The expanded configuration may thus be referred to as “trapping configuration”, and the device may be referred to as a “trapping device”. In alternative embodiments, the device is configured for allowing passage (i.e., not blocking) such type of objects (i.e., objects of the same dimensions as the cooperating object of the former embodiments). In other words, the device has no or reduced trapping capability.

The devices and methods discussed herein may thus provide retentive devices or systems that are useful for local drug release or other medical purposes, based on systems with expanding geometry through unfolding. The systems are initially in a condition or configuration suitable for swallowing. Then, the system may expand (i.e., unfold) in the predetermined location of the GI tract to prevent being released from the location. Eventually, the system form may reduce in size to move part the predetermined region (e.g., by passing pylorus valve or ileocecal valve) or disassemble or disintegrate. The devices and methods discussed herein may provide the following aspects: rapidly expand in a sufficiently short time; stay in the predetermined location for a predetermined residence time period, e.g., such that releasing of the drug and/or the imagine/measurements can be efficiently achieved; and maintain a sufficiently large size throughout the residence time period under GI or physiological conditions under fasted or fed states. The devices and methods discussed herein may provide such aspects throughout a relatively long shelf-time. The devices and methods discussed herein may comprise an emergency release mechanism to expel the delivery system or (e.g., trapped) dosage form in an emergency situation.

The expression “carrying a load of an active pharmaceutical ingredient” as used in the present disclosure refers to the fact that the device may optionally securely contain a quantity of said API in the state of the device prior to administration (e.g., the closed configuration). By “securely”, it is meant that prior to administration (e.g., the closed configuration), the device securely contains a quantity of said API located inside a convex hull of the device, and said API is not extractible unless deforming the outer shape of the device, due to physical barriers formed all around the API by components and/or material. In other words, the device may carry an embedded dosage form including an API, i.e., a therapeutic dosage form. Thus, the device is enabled to be administered and securely carry/transport with itself a certain amount of the API to the predetermined location. Then, when the device is in the expanded configuration, the device is configured for releasing at least part (e.g., all of) said (initially carried) load of the API. Indeed, the device may be configured for the API to be exposed to chyme flow so as to release the API, at least when the device is in the expanded configuration and positioned at the predetermined location. The release may optionally be over a certain period of time, e.g., longer than one minute, ten minutes, thirty minutes, one hour, two hours, five hours, twelve hours, one day, or one week. In other words, the API load may be configured to erode, diffuse, or dissolve from within the device.

The device may present a structural rigidity substantially independent of the load of the API. The device may notably maintain its expanded shape while the API is being released, and in particular even if the API has been fully released. In particular, the device may be configured such that, when administered to a subject but without any load of API, the device is structurally rigid enough to be retained at the predetermined location of the GI tract of the subject. As such, the load of the API may be unsupportive of the device's structure.

The device may carry a load of the API in any manner, and optionally in several different manners. The device may embed at least part of the load of the API inside (e.g., enclosed in) or on (e.g., mounted onto or attached to) one of its components. Said at least part of the load may remain embedded in the device. In other words, said at least part of the load may be securely attached to the device even in the expanded configuration, such that said at least part of the load is not extractible (apart from release of the API) unless exerting a force above a predetermined threshold. Alternatively or additionally, at least part of the carried load may be detachable from the device when the device deforms into the expanded configuration.

The load of API may be any type of formulation of the API, and it may comprise not only the API, but optionally also one or more pharmaceutically acceptable excipients and optionally inactive excipients including one or more of filler, binder, lubricant, diluent, preservative and the like.

The device may for example comprise an inner (available) space in the closed configuration. The inner space may contain (e.g., lodged therein and/or unattached) at least part of the load of the API. The inner space may be an interstice left between components of the device. The inner space may be an interstice left (i.e., present) between or flanked by components of the device. Such examples maximize space left between components of the device by using it for API loading. In examples, the device may comprise at least two flexible arms (e.g., at least three or at least four), each arm having a first end and a second end, a first and second coupling heads, wherein the first end of each arm is coupled to the first coupling head and the second end of each arm is coupled to the second coupling head, and a resiliently deformable member configured to force the coupling heads together to bend the arms thereby biasing the device in the expanded configuration. In such examples, the inner space may be formed in the closed configuration around the resiliently deformable member the optional and between the arms and the coupling heads.

The device may further comprise a locking assembly configured, when activated, to maintain the device in the expanded configuration. Said locking assembly is configured to be activated upon transfer of the device into the expanded configuration. For example, the locking assembly may comprise a locking member. In such examples, the inner space may be formed in the closed configuration around the resiliently deformable member and/or the optional locking assembly or member, and between the arms and the coupling heads.

Alternatively or additionally, at least one material portion or at least one component of the device may comprise an exposed cavity (i.e., a substantially void space inside a portion of material and having a peripheral wall substantially enclosing the space). In such a case, the exposed cavity may contain (e.g., enclosed therein and/or unattached) at least part of the load of the API. The cavity is open to the device environment, at least when the device is in the expanded configuration. Thus, the load of the API may flow/erode from the cavity when the device is in the expanded configuration. The cavity may be formed within a peripheral wall having apertures which provide exposure of the exposed cavity. Such examples allow simple manufacturing and fine control of the API release. In particular, the apertures may present a design which provides a predetermined release rate. Alternatively or additionally, the exposure apertures may be coated, so as to expose the contained API load only when desired, or on the contrary uncoated. The coating forms an API release delay mechanism. In examples, the device may comprise at least two flexible arms (e.g., at least three or at least four), and one or more (e.g., all) of the arms may comprise such cavity (e.g., within one or more—e.g., each—arm section when the arm is articulated and/or composed of a set arm sections).

Alternatively or additionally, at least one material portion or at least one component of the device may comprise an exposed recess. In such a case, the exposed recess may lodge (i.e., be filled with) at least part of the load of the API (e.g., press-fitted in the recess, or formed by molding inside the recess). Such examples allow simple manufacturing. Alternatively or additionally, the arms may comprise exposed recesses. Each exposed recess may be coated at its opening, so as to expose the contained API load only when desired, or on the contrary uncoated. The coating forms an API release delay mechanism.

Alternatively or additionally, at least one material portion or at least one component of the device may comprise a coating on an exposed surface, the coating containing at least part of the load of the API. For example, each arm section and/or a support tube may be coated with any mixture containing the API.

The load of the API may present any texture, shape, and/or composition. For example, the API may be contained in a solid form, in a semi-solid form, as powder, as a gel texture, and/or in a liquid. In addition, the API may be contained in one texture, shape, and/or composition at one location, and in another texture, shape, and/or composition at another location. Furthermore, the device may carry several APIs, for example cooperating together to treat a medical condition. The load of the API may contain one or more pharmaceutically acceptable excipients. The one or more excipients may be inactive and/or include one or more of filler, binder, lubricant, diluent, preservative, control release agent, disintegrant (e.g., sodium, starch, glycolate) and the like. Solid forms of the API may include one or more tablets, and/or pellets. The carried tablets (e.g., pills) may have common tablet shapes such as round, standard convex, compound cup, oval, bullet, triangle, diamond, etc., so as to fit the cavity, recess, groove, inner space designed to accommodate the tablet. The solid forms may be coated or uncoated. The coating may form an API release delay mechanism.

The device may be unable to release the API (at all) when the device is in the closed configuration. The device may further be optionally unable to release the API unless the device is in the expanded configuration. For example, in some embodiments of the present disclosure the device may comprise a container in which the device can be fitted in the closed configuration, and the container may form a physical barrier preventing release of the API carried by the device prior to the container dissolving and the device thereby transferring from the closed configuration into the expanded configuration. In some embodiments, the device includes an API useful in treating a disease or disorder for which dispensing of the API at the predetermined location (e.g., the gastric region or the ICV region) is beneficial. The device may carry any quantity of the API. For example, the load of the API (e.g., possibly including excipients) may occupy at least 5%, 10%, 15%, 20%, or 25% of the volume of a convex hull of the device in the closed configuration. In other words, the API may be contained in an embedded solid form or formulation, and the form/formulation, and the device may offer in the closed configuration enough free space such that at least 5%, 10%, 15%, 20%, or 25% of the volume of its convex hull is occupied by (accommodated with) the form/formulation. The type and quantity of API load may be determined by a healthcare professional.

The release rate of the API by the device may be easily configurable, for example via the formulation of the load of API (e.g., with specific excipients thereof), and/or via specific dimensioning, shaping and/or coating of the exposure apertures and/or exposed cavity. The device thus forms a particularly easily adaptable platform, allowing a high degree of freedom in release rate and control thereof, quantity of API, and/or type of API. Numerous examples are thereby illustrated herein in reference to the figures and the experimentals.

The term “dosage form” as used in the present disclosure refers to solid dosage forms which may include an API. Some dosage forms according to the present disclosure may include an API (and optionally one or more pharmaceutically acceptable excipients) and may be referred to as a “therapeutic dosage form”. In some embodiments, wherein the predetermined location is the ICV region, the dosage form includes an API useful in treating a disease or disorder for which dispensing of the API at the ICV is beneficial. The dosage form may include the same API as the load carried by the device, or a different API.

Some dosage forms according to the present disclosure, and in particular wherein the predetermined location is the gastric region, may not include an API but may, for example, instead include an emptying agent for modifying the environmental conditions at the location of the GI tract to cause the device according to some embodiments of the present disclosure to transfer from the expanded configuration into the emptying configuration. Such dosage forms may be referred to as “emptying dosage forms”. In some embodiments, the emptying agent is a pharmaceutically acceptable ingredient in an amount effective to cause transfer of the device into the emptying configuration. In some embodiments, wherein the predetermined location is the ICV region, the emptying agent is an acidic ingredient including citric acid, tartaric acid and the like. In some embodiments, wherein the predetermined location is the gastric region, the emptying agent is a basic ingredient including aluminum hydroxide, magnesium carbonate, calcium carbonate sodium bicarbonate and the like.

The dosage forms (comprising API or emptying agent) may include, for example, tablets, pellets or capsules. Tablets may have common tablet shapes such as round, standard convex, compound cup, oval, bullet, triangle, diamond, etc. Capsules may carry a solid (e.g., tablet, particles, granulates) or liquid load. Dosage forms generally have dimensions such as to fit into a cylinder having a length from 10 mm to 30 mm and a diameter from 7 mm to 12 mm. In some embodiments, the dosage form comprises a mixture of active ingredient(s) (API or emptying agent) and inactive excipients including one or more of filler, binder, lubricant, diluent, preservative and the like.

In some embodiments the dosage form may present any form that can be dosed to the subject, such as a gel, a solid, a semi-solid, or a liquid form, or any combination thereof. In some embodiments, the emptying dosage form may be an emptying tablet, that is, an oral dosage form for cooperation with the device to trigger emptying, when the trigger assembly is configured to activate when the device in the expanded configuration is exposed to a surrounding environment of a pH being above or below a predetermined threshold of, for example pH above 6 or below 3. The emptying dosage form may be designed to transit to the predetermined location of the GI tract, be retained at the location, release its basic or acidic payload (emptying agent) thereby increasing/decreasing the surrounding environment pH, enabling activation of a trigger assembly of the device (e.g., erosion of—e.g., enteric polymers such as Eudragit® (methacrylic acid-methyl methacrylate copolymer) type L or S or combination of or HPMC (hydroxypropyl methylcellulose) acetate succinate (HPMC-AS) L grade (LG)—pins as support elements) and causing transfer of the device into the emptying configuration. In embodiments wherein the predetermined location is the gastric region an exemplary emptying tablet composition includes a basifying agent (to increase the pH above a threshold pH).

In embodiments wherein the predetermined location is the ICV region, the dosage form comprises at least one coating, preferably a pH dependent coating that enables delivery of a substantially intact dosage form to the ICV region.

“Substantially intact” may refer to any dosage form herein, in particular in the ICV case, being at least capable of being blocked by the trapping device when reaching the ICV. Substantially intact may also refer to the dosage form having undergone less than 50%, less than 40%, less than 30%, 20%, less than 15% less than 10% or less than 5% downsizing before reaching the ICV and/or to the dosage form having released less than 50%, less than 40%, less than 30%, 20%, less than 15% less than 10% or less than 5% of the API or of the emptying agent before reaching the ICV. In some embodiments the dosage form includes an external coating comprising one or more enteric polymer. In some embodiments the dosage form includes one or more pH dependent coating. In some embodiments, the dosage form includes a time dependent coating such as HPMC and or ethyl cellulose and optionally a pore forming agent. In some embodiments, the dosage form is configured to begin to downsize and/or release API or the emptying agent in the ICV region between 72 hours and 4 hours post administration. In some embodiments, the dosage form is configured to downsize and or release API in the ICV region 72 hours after administration, 60 hours after administration, 48 hours after administration, 36 hours after administration, 30 hours after administration, 24 hours after administration, 18 hours after administration, 15 hours after administration, 12 hours after administration, 11 hours after administration, 10 hours after administration, 9 hours after administration, 8 hours after administration, 7 hours after administration, 6 hours after administration, 5 hours after administration or 4 hours after administration or 3 hours after administration.

The term “external envelope” may be used to refer to a hull separating internal and external portions of the device. For example, the external envelope of the device may comprise an outer surface of the device.

The term “pharmaceutically acceptable” refers to a material that is not biologically or otherwise unacceptable when used for the purposes of the present disclosure. For example, the term “pharmaceutically acceptable carrier” refers to a material that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration. The materials used for manufacturing the device in accordance with the present disclosure may be pharmaceutically acceptable.

The term “pH dependent polymer” may refer generally to a polymer whose degradability or dissolubility changes depending on the pH of a surrounding solution. For example, in some embodiments of the present disclosure, a trigger assembly may include one or more support elements which may be at least partially made of a pH dependent polymer so that the one or more support elements do not degrade at standard environmental conditions at the location of the GI tract (i.e., stomach standard pH of about 1-3 or 1-4 or ICV standard pH of about 6.8-7.5) while do degrade at a less or more acidic pH. A type of pH dependent polymer is an enteric polymer. An enteric polymer may be understood as a polymer that does not readily dissolve or degrade under the typical pH and other physical conditions of a human stomach, but that does dissolve or degrade at pH and other physical conditions of the intestinal tract of a human, i.e., the conditions that exist following passage from the stomach through the pylorus (i.e., pH>5). For example, in some embodiments of the present disclosure the device may comprise a container in which the device can be fitted in the closed configuration and the container may dissolve in the predetermined location. In some embodiments, as described below, the trigger assembly may include one or more support elements at least partially made of an enteric polymer or of a pH dependent polymer. When the singular form of “pH dependent polymer” is used, this can refer to one enteric polymer, a mixture of two or more enteric polymers, or a mixture of polymers of which at least one is a pH dependent polymer, as long as the resulting mixture is pH dependent in nature.

The timer can be made of, for example, a polymer that gradually degrades in the gastric environment, such “time dependent” timer can be made of time dependent polymers such as HPMC or of Eudragit® EPO (butyl methacrylate, dimethylaminoethyl methacrylate, methyl methacrylate copolymer) that are soluble in the gastric milieu (low pH).

The optional load of API carried by the device may be distinct and separate from the support elements (e.g., timers). In some embodiments, the load of API carried by the device is distinct and separate from the support elements (e.g., timers). Each support element may be integrally formed, and the load of the API may be elsewhere. In examples, the support elements may be located inside the coupling heads, while the carried load of the API may be located between the coupling heads and the arms (i.e., outside the coupling heads). Optionally, the support elements may comprise none of the API. In some embodiments, the support elements (i.e. timer(s)) comprise no API. In variations, the device may comprise one or more integrally formed blocks both forming a support element and carrying at least part of the load of the API. While releasing the API, the blocks erode to a point of degradation, thus triggering emptying of the device. With regard to the timer, the device includes at least one timer mechanism. The timer may have dimensions of about 1.5 mm diameter by 6 mm length, which uses minimal space within the device, thereby enabling high drug loading. The timer mechanism is sufficiently strong to support the device in the expanded configuration and can resist a frequent folding force of up to 1.5 Kg (about 14.7 N) while the platform is in the unfolded (expanded) configuration, enabling gastric retention for extended periods of time (96 hrs in vitro retention and more than 48 hr retention in dog stomach) and quick transformation to the disassembly configuration (e.g. after timer has eroded) thereby enabling gastric emptying at the end of the predefined GR time. Gastric retention requires physical resistance of the platform to gastric emptying forces, which are known to be even stronger in dog compared to human. In some embodiments, the timer may be designed to fit within the head or heads of the platform. In alternative embodiments, it is contemplated that the timer could be located in the arms of the platform, for example in the pins of the articulated arms, which will be discussed further below.

In some embodiments the device includes more than one timer, and may have for example dual timers. In some embodiments, two timers are provided in the device and may provide a safety net in the event that one of the timers is not able to function as desired. For example, the timer may include a first timer configured to dissolve at a first location within the GI tract, while the second timer can be configured to dissolve at a second location within the GI tract. The addition of the second timer can act as a safety mechanism. For example, when the platform is targeted for retention in the stomach, the first timer is designed to gradually dissolve in the stomach while the second timer is designed to gradually dissolve in the intestine. In an unexpected event that the device is prematurely transitioned into the small intestine while in the unfolded, expanded configuration, the second timer will erode and the platform will collapse into the emptying configuration for safe exiting from the body.

Local release of therapeutic agents in the gastric region may have therapeutic benefit when used in various medical conditions. Non-limiting examples include:
a. Treatment of a gastric ulcer, or gastroesophageal reflux disease. Effective treatment may be achieved when local and durable the drug in the stomach is obtained (i.e., high local concentration and long exposure duration).
b. Treatment of other stomach disease, including stomach cancer and gastric microbial diseases. Examples of stomach cancer drugs include including angiogenesis inhibitors, check point inhibitors, metabolite inhibitors etc. Stomach infections may be caused, for example, by parasites, bacteria (such as H. pylori, viruses, and fungi. Useful anti-microbials include amoxycillin neomycin, rifaximin, anthracyclines, bleomycins, mitomycin, dactinomycin, and plicamycin. A health care professional may identify a relevant antibiotic for a particular disease, from for example, the WHO publication “Critically important antimicrobials for human medicine”, (ISBN: 978-92-4-151552-8; 6th revision).
c. Improved systemic therapeutic exposure of the APIs (e.g., absorption and bioavailability) which may enable achieving systemic plasma concentrations having minimal fluctuations enabling better efficacy and safety. For example, to improve absorption in drugs having short absorption window such as dopa agonists for Parkinson's or to lower risk of adverse effect in drugs having low therapeutic index such as anti-cancer (ibrutinib) and steroids.
d. Improvement in patient adherence in which the device may be designed to be retained for prolonged time in the stomach while releasing API. Such treatments lower the amount of drug dosing per day and improve the adherence to treatment for both the patient and the care givers (for example, in anti-psychotic drugs, anti HIV medications, etc.).

Local release of therapeutic agents to the ICV and ascending colon may have therapeutic benefit when used in various medical conditions. Non limiting examples include:
a. Treatment of Inflammatory Bowel Disease (IBD) including Ulcerative Colitis (UC) and Crohn's disease (CD). The ileocecal junction and ascending colon are inflammation sites (especially in pancolitis). Effective treatment may be achieved when local and/or topical exposure of the drug in the inflamed tissue is obtained (i.e., high local concentration and long exposure duration). Examples of useful APIs include steroids (budesonide), mesalamine, 6-mercapto-purine, etc.
b. Treatment of colon disease, including colon cancer and colonic microbial diseases. Examples of colonic cancer drugs include including angiogenesis inhibitors, check point inhibitors, metabolite inhibitors etc. Drugs currently approved in the US for treating colon cancer, include Irinotecan Hydrochloride, Capecitabine, Oxaliplatin, Erbitux, Fluorouracil, Leucovorin Calcium, Irinotecan Hydrochloride, Trifluridine, Tipiracil Hydrochloride, Regorafenib, Capecitabine, Ziv-Aflibercept. Colon infections may be caused, for example, by parasites, bacteria, viruses, fungi. Useful anti-microbials include amoxycillin neomycin, rifaximin, anthracyclines, bleomycins, mitomycin, dactinomycin, and plicamycin. A health care professional may identify a relevant antibiotic for a particular disease, from for example, the WHO publication “Critically important antimicrobials for human medicine”, (ISBN: 978-92-4-151552-8; 6th revision).
c. Improvement of absorption and bioavailability of therapeutic agents; for example, achieving therapeutic systemic plasma concentrations having minimal fluctuations to better enable efficacy and safety. For example, Vitamin B (optionally given with absorption enhancer) which is known to be absorbed mainly in the ileocecal junction. Another example is GLP-1 receptor agonists given with absorption enhancer such as SNAC to be released and absorbed in the targeted site (ICV). Further examples include esterified levodopa (eLD), to improve the systemic exposure compared to LD. Actually, experiments have shown that when eLD is given in a standard oral dosage form (targeted to the upper GI tract) although eLD has a better solubility and passive permeability profile, no significant improvement in systemic exposure is achieved. This is likely due to extensive pre-systemic metabolism of eLD into LD. Therefore, local release of eLD in using devices and methods of the present disclosure to the ICV region, which has lower esterase activity than the upper GI tract, may enable better systemic exposure and enhance the therapeutic advantage of eLD (Laizure et al, Pharmacother. 2013; 33(2): 210-222; Itoh et al, J Pharma Sci, 99:1, 2010; Fix et at, Pharmaceutical Res, 6:6 1989). Additionally, targeting the ICV region may have significant advantage over the current oral LD treatment. Actually, in the proximal intestine, LD absorption is carried out by an amino acid transporter, which is in competition with food originating amino acids. By contrast, in the terminal ileum the absorption of eLD is mostly passive and no interference is expected, leading to a more stable exposure of drug. Further examples also include Vitamin B12 which is known to be poorly absorbed in the small intestine and known to be absorbed mainly in the ileocecal region. Drugs that can benefit from prolonged retention and release in the ICV region include other esterified drugs including Tenofovir disoproxil, Adefovir dipivoxil, Prasugrel and the like. Tenofovir disoproxil is given as ester prodrug since it has much better absorption compared to the drug itself. Still, the bioavailability of the prodrug is only about 30% due to extensive intestinal esterase degradation. By targeting Tenofovir disoproxil to ICV region having lower esterase activity (Van Gelder, et al. (2000). Species-dependent and site-specific intestinal metabolism of ester prodrugs. Int J of Pharmac. 205. 93-100.) a more consistent absorption and bioavailability may be achieved.
d. Changing the administration site of known drugs. In general, there are examples of drugs, that upon changing the administration site were found to be beneficial for different, new indications. For example, Copaxone ° (glatiramer acetate), which is normally given subcutaneously for the treatment of multiple sclerosis has been shown to be effective in treating UC when given topically (see Yunliang Yao et al., “Glatiramer acetate ameliorates inflammatory bowel disease in mice through the induction of Qa-1-restricted CD8+ regulatory cells”, Eu Jl Immunol, 43:1, pg 125-136). The present disclosure, which introduces a new site of drug deposition and or absorption holds a potential for treating a variety of indications using APIs not necessarily intended for that particular use (e.g., administration at the ICV or upper colon).

Examples of the API contained in the optional load carried by the device are now discussed and/or contained in the optional cooperating therapeutic dosage form (in ICV use case where the device has a trapping capability). In some embodiments, the API is a small molecule. In some embodiments, the API is a prodrug. In some embodiments, the API is a pharmaceutically acceptable salt of an API. In some embodiments, the API comprises amino acid or nucleotides. In some embodiments, the therapeutic agent may be a combination of two or more therapeutic agents. A non-limiting list of APIs includes anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs, e.g., naproxen); antibacterial agents; anti-fungal agents; sepsis treatments; steroids; local or general anesthetics; monoamine oxidase inhibitors; hypothalamic phospholipids; endothelin converting enzyme (ECE) inhibitors; opioids; thromboxane receptor antagonists; potassium channel openers; thrombin inhibitors; growth factor inhibitors (e.g., modifiers of EGF, PDGF activity); anti-cytokines (e.g., anti-TNF activity); anti-platelet agents (e.g., aspirin); anticoagulants; heparins; renin inhibitors; neutral endopeptidase (NEP) inhibitors; vasopepsidase inhibitors (dual NEP-ACE inhibitors); HMG CoA reductase inhibitors (e.g., statins); squalene synthetase inhibitors; fibrates; bile acid sequestrants; anti-atherosclerotic agents; MTP Inhibitors; calcium channel blockers; potassium channel activators; alpha-muscarinic agents; beta-muscarinic agents; antiarrhythmic agents; diuretics; thrombolytic agents; anti-diabetic agents; mineralocorticoid receptor antagonists; aP2 inhibitors; phosphodiesterase inhibitors; protein tyrosine kinase inhibitors; antiproliferatives; immunosuppressants; antimetabolites; antibiotics; enzymes; farnesyl-protein transferase inhibitors; hormonal agents (e.g., cortisone); microtubule-disrupter agents; plant-derived products (e.g., taxanes); topoisomerase inhibitors; prenyl-protein transferase inhibitors; cyclosporins. Listings of additional examples of known therapeutic agents can be found, for example, in the United States Pharmacopeia (USP), Physician's Desk Reference (Thomson Publishing), and/or The Merck Manual of Diagnosis and Therapy, 20th ed. (2018) Robert S. Porter, ed., Merck Publishing Group, or, in the case of animals, The Merck Veterinary Manual, 11th ed., Susan E. Aiello and Michael A. Moses eds., Merck Publishing Group, 2016; and “Approved Drug Products with Therapeutic Equivalence and Evaluations,” published by the United States Food and Drug Administration (F.D.A.) (the “Orange Book”).

In embodiments wherein the predetermined location is the gastric region, an API may include any substance relevant for gastric retention as recognized in the art which may be therapeutic, diagnostic or otherwise beneficial. A list of relevant APIs may comprise APIs which act locally in the stomach, APIs with primarily absorption in the stomach, and APIs which are poorly soluble in alkaline pH. The list may further comprise APIs with narrow windows of absorption, APIs with rapid absorption from the GI tract, APIs that degrade in the colon, and APIs that disturb colonic microbes.

Further provided herein is a method of treating a condition which may benefit from local dispensing of an API at the location of the GI tract of a subject suffering from the condition. The treatment includes orally administering to the subject a device as described herein, thereby treating said condition in said subject.

“Treating” as used herein encompasses, e.g., inducing inhibition, regression, clinical remission or stasis or inhibiting, reducing the severity of, eliminating or substantially eliminating, or ameliorating a symptom of the disease or disorder, e.g., a gastric ulcer, and gastroesophageal reflux disease, alleviating, at least partially, Parkinson's disease related symptoms.

“Inhibition” of disease progression or disease complication in a subject means preventing or reducing the disease progression and/or disease complication in the subject.

A “symptom” associated with a disease or disorder disclosed herein includes any clinical or laboratory manifestation associated with any relevant disease or disorder and is not limited to what the subject can feel or observe. A non-limiting example includes the following for gastric ulcers: weight loss diarrhea, nauseas, vomiting, abdominal cramps and aches, abdominal pain, inflammation, tiredness, anemia, perforations. A non-limiting example includes the following symptoms for IBD: weight loss, macroscopic colonic damage, colonic ulceration, intestinal and/or peritoneal adhesion, diarrhea, bowel wall thickening, nauseas, vomiting, abdominal cramps, abdominal pain, intestinal bleeding, intestinal inflammation, GI tract inflammation, rectal bleeding, tiredness, anemia, fistulae, perforations, obstruction of the bowel or any combination thereof. Symptoms of other conditions are known in the art and are identified by a medical practitioner.

The device according to the present disclosure may have any physical configuration that is compatible with certain basic functionalities.

First, it is configured for being administered to a human. For example, the device can be deformed (e.g., folded) from an expanded configuration into a closed configuration and locked by a locking element (e.g., a capsule) so as to be sized and shaped for oral administration i.e., ingestion. The locking element may substantially maintain the device integrity prior to entry at the predetermined location. The locking element may dissolve after (e.g., within 1 or 10 minutes, or with 1 hour) exposure to environmental conditions at the location of the GI tract, for example the stomach environmental condition or small intestine condition. The device may also, in some embodiments, be configured to be positioned by endoscopy i.e., by gastroscope in the stomach. The device may also, in some embodiments, be configured to be positioned by endoscopy—i.e., upper endoscopy or colonoscopy—at the ileocecal valve in the trapping configuration. In some embodiments, the device in the collapsed configuration may be manipulated with the endoscope and expanded at the predetermined location. In some endoscopically administered embodiments, the device may be inflatable between a collapsed/deflated configuration and the expanded configuration. The device may be positioned at the location in the collapsed configuration and inflated in the expanded configuration thereafter. In some upper endoscopy administered embodiments as well as in some orally administered (ingested) embodiments, the device may be administered to the subject under fasted conditions.

Second, in orally administered/ingestible embodiments, the device is configured for being capable of transiting through the GI until the predetermined location, for example the stomach or the ileocecal valve. For example, the device may be contained in a capsule which does not dissolve before exposure in standard environmental conditions (fasted and/or fed) of the predetermined location. In examples where the predetermined location is the stomach, the device in the expanded configuration may be configured to enable chyme flow into the small intestine while present in the stomach. In examples where the predetermined location is the ICV region, the device in the expanded configuration may be configured to enable chyme flow in the small intestine while transiting to the ileocecal valve. Furthermore, the device may also be configured to position at or in proximity of the ileocecal valve due to the subject GI motility.

Third, the device is configured to transfer from the closed configuration into the expanded configuration due to a biasing assembly. The device is then configured in the expanded configuration is configured for retention in the predetermined location in standard GI motility conditions, for example in the stomach (i.e., not to pass through the pyloric valve) or at (i.e., directly before, proximal to) the ileocecal valve (i.e., not to pass through the ileocecal valve). For example, the device in the expanded configuration may be sized and/or shaped and/or have a structural rigidity that prevents passage through the pyloric valve or the ileocecal valve of a standard subject under standard GI motility conditions.

Fourth, the device is configured for being capable of transferring into an emptying configuration which allows passage through the location of the GI tract (e.g., through the pyloric valve or the ileocecal valve), and optional excretion through the subject's body. For example, the device may include a trigger assembly causing the device to transfer into the emptying configuration when activated. The trigger assembly may be configured to be activated when the device in the expanded configuration is exposed to an activation signal such as being exposed to standard environmental conditions at the location of the GI tract for a predetermined residence time period or such as a surrounding environment reaching a predetermined set of environmental conditions (e.g., a pH threshold). The device may be configured to include one or more structural weak points which may cause the device to swiftly fall apart into the emptying configuration when the trigger assembly is activated.

Fifth, the device comprises means for limiting size and use of material of the biasing assembly, and/or extending shelf-life. For example, the device may comprise a locking assembly configured, when activated, to maintain the device in the expanded configuration. The locking assembly may be configured to be activated upon transfer of the device into the expanded configuration. For example, the locking assembly may comprise a snap-fitting or a latching mechanism. For instance, the locking assembly may comprise at least one locking member configured for being snapped into another part of the device upon expansion of the device. Thus, the locking assembly may relieve the biasing assembly and provide at least substantially the force necessary to retain the device in the expanded configuration. In turn, the locking assembly need not be made of a material allowing resilience such as metal or silicone. Additionally or alternatively, the biasing assembly may have a strained state and a relaxed state. The biasing assembly may be configured, while the device is in the closed configuration, to transfer from the relaxed state into the strained state. The biasing assembly may be in the strained state capable of transferring the device from the closed configuration into the expanded configuration. In such a case, the biasing assembly is preserved during shelf-life, such that it can present a lower unfolding force at the time of manufacturing and still be functional at the time of use, after storage time has passed, such as months, a year or even two years.

In some embodiments, the device may be used in conjunction with another means able to trigger the device to transfer into an emptying configuration. For example, the means may comprise an emptying dosage form. Alternatively, the means may comprise an oral lead such as a catheter or endoscope that can be placed close to the predetermined location (e.g., pylorus region or ileocecal valve). The oral lead may deploy an amount of an emptying agent sufficient to cause the environment of the predetermined location to reach at least one activation environmental condition thereby causing activation of the trigger assembly and transfer of the device into the emptying configuration. In examples of this embodiment, the means can be used in case of emergency, e.g., when the pyloric valve or ileocecal valve is obstructed.

Optionally, the device in the expanded configuration may be configured to release at least partially carried API, thus enabling a retained treatment within the predetermined location of a desired duration. The device may allow chyme flow (e.g., through the pyloric valve or ileocecal valve) while releasing at least partially the carried API.

In particular, in embodiments where the device comprises at least two (e.g., at least three or at least four) flexible arms, two coupling heads, and a resiliently deformable member, flexible arms, coupling heads, and resiliently deformable member may be dimensioned and/or arranged to allow chyme flow in an inside space, e.g., formed around the resiliently deformable member between the arms and the coupling heads, while the API is being released. The chyme flow in the inside space may improve the release of the API by providing a more uniform diffusion for the API. In particular, in such embodiments, the device may carry/embed the API such that it is exposed at least to said inside space. According to preferred embodiments, the device may comprise at least three arms, even more preferably four arms. This imparts a 3D shape to the device in the expanded configuration, thus achieving high retention capability.

Furthermore, the device according to some embodiments of the disclosure may have in the expanded configuration a convex hull with a high sphericity (e.g., larger than 0.8 for example in some embodiments about 0.85 to about 1) and/or a low ratio between a maximal circumference and a minimal circumference (e.g., below 1.5, for example in some embodiments about 1 to about 1.2). This high sphericity of the expanded structure improves tissue pressure distribution. The device according to the present disclosure may have a particularly high (convex hull) volume ratio of the expanded configuration (i.e., the form in which the device is retained in the stomach or ICV region) relative to the closed configuration (i.e., the form in which the device is administrated). This high ratio enables the retainability of the device at the predetermined location while enabling easier swallowability.

The device according to the present disclosure may be simple to manufacture, and its design (e.g., the arms, the coupling heads, and the resiliently deformable member, shock-absorption capability of the locking member) may provide some flexibility while it is in the expanded configuration, so as to avoid damaging the body tissue. In addition, the device may be relatively fast to expand due to such a configuration which increases the probability of retention following dosing under fasted gastric condition.

In embodiments wherein the predetermined location is the ICV region, the device according to the present disclosure may have any physical configuration that is compatible with the additional functionality of blocking (trapping) cooperating rigid objects (e.g., dosage forms) having predetermined external dimensions. Trapping of dosage forms by the device may enable further dispensing locally a drug in the ileocecal region. In general, cooperating objects may have one or more of the following features:
i. Be configured for ingestion i.e., made of pharmaceutically acceptable material(s) and have swallowable dimensions.
ii. Have a hull substantially occupying a volume larger than a cylinder of a length of 7 mm and a diameter of 8 mm.
iii. Have a hull substantially occupying a volume larger than a sphere of 7 or 8 mm diameter.
iv. Embed an active agent (therapeutic agent or an emptying agent). The therapeutic agent or emptying agent being configured to be (exclusively or at least mainly) released at the ICV.s
v. Be capable of downsizing so as to pass through the trapping device after the embedded therapeutic agent or emptying agent is significantly released.

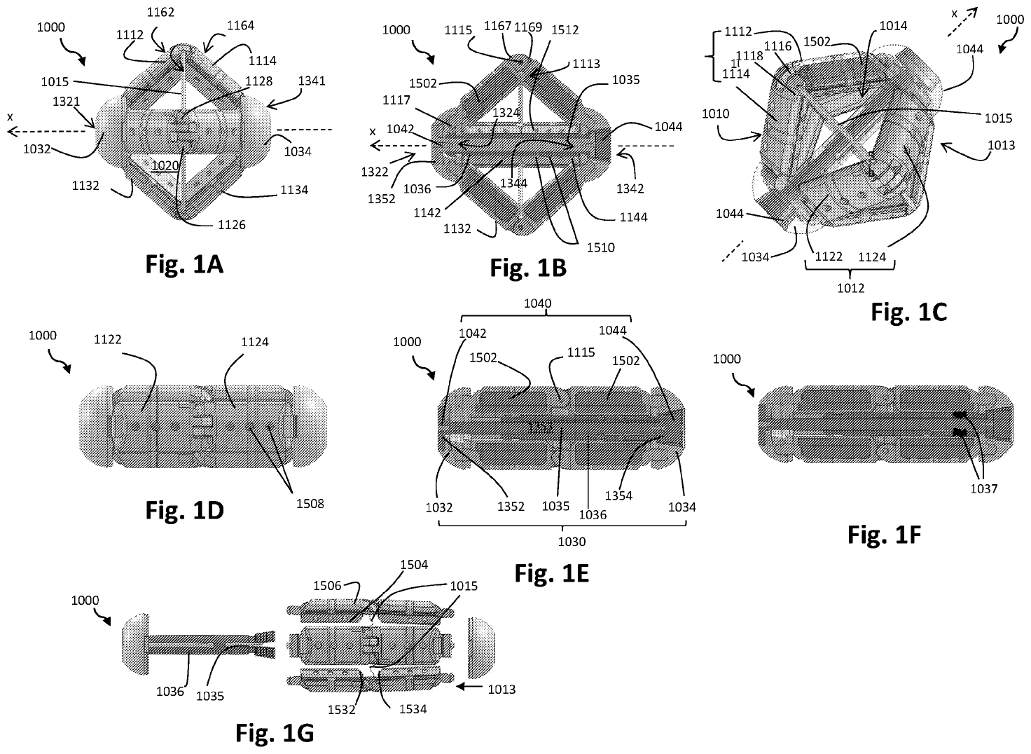

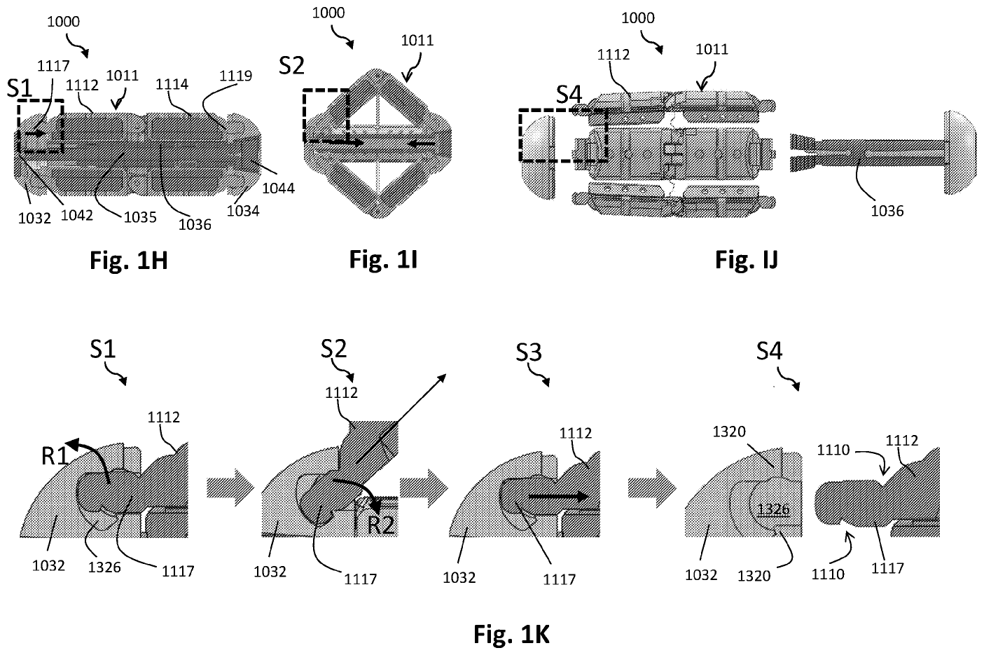

FIGS. 1A-1C, FIGS. 1D-1F, and FIG. 1G show an example of a device 1000 for temporary GI tract retention according to the present disclosure, respectively in an expanded (open) configuration (FIGS. 1A-1C), in a closed (swallowing) configuration (FIGS. 1D-1F), and in a disassembled configuration which may form an emptying configuration and/or a pre-assembling configuration during manufacturing (FIG. 1G). FIGS. 1A, 1D, and 1G show a front view of the device 1000. FIG. 1C shows a perspective view of the device 1000 in the same configuration as in FIGS. 1A, 1B, 1E and IF show a longitudinal cross-section view (along axis X) of the device 1000 in the same configuration respectively as in FIGS. 1A and 1D, the cross-section being taken in a median plane of the device 1000 parallel to the view plane of respectively FIGS. 1A and 1D.

The device 1000 may be deformable from the closed configuration of FIGS. 1D-1F into the expanded configuration of FIGS. 1A-1C, and the device 1000 may be irreversibly disassembled from the expanded configuration of FIGS. 1A-1C into an emptying configuration identical or similar to the disassembled configuration of FIG. 1G. The device 1000 is intended to be orally administered—in the closed configuration—to a patient for temporary residence—in the expanded configuration—at the predetermined location of the GI tract, for example at the stomach or at the ileocecal valve of said patient. In some embodiments, the device in the closed configuration may be capable of passing through the predetermined location, for example by passing through the pyloric valve or the ileocecal valve of the patient. In some embodiments, the external envelope of the device in the closed configuration has dimensions enabling moving past the predetermined location, for example passage through the pyloric valve or ileocecal valve and the device in the emptying configuration has an external envelope similar to the device in the closed configuration. Basically, the device in the closed configuration may be capable of being fitted into a cylinder (for example a capsule shell)of about 35 mm length or smaller and of about 12 mm diameter or smaller, for example in a cylinder of 30 mm in length and of 10 mm or 9 mm in diameter or 27 mm in length and about 8.4 mm in diameter.

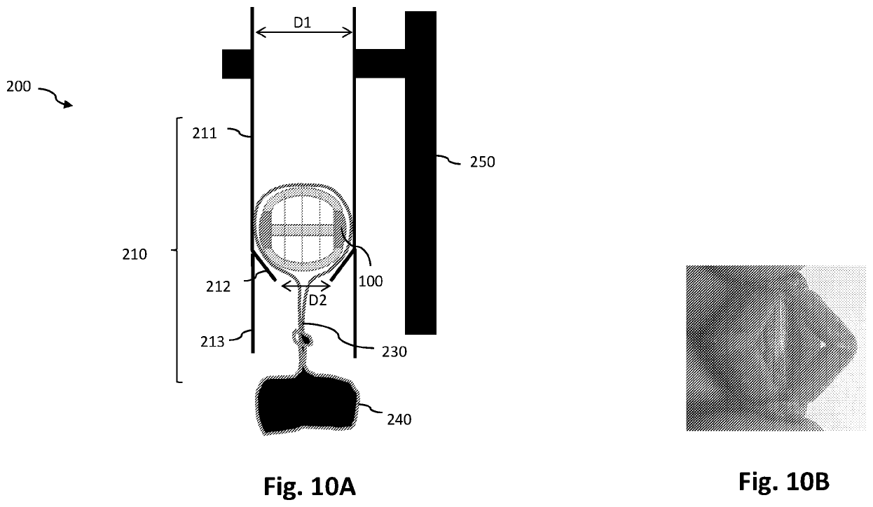

In the expanded configuration, the device 1000 is configured to be retained at the predetermined location and resist standard GI motility. The device 1000 may include a flexible frame. The device 1000 in the expanded configuration may have an uncompact shape while the device in the closed configuration may have a compact shape. In some embodiments, the dimensions of the device in the expanded configuration may be such that the device cannot pass through an orifice of about 17 mm diameter and preferably of about 20 mm. In particular when the predetermined location is the stomach region, the dimensions of the device in the expanded configuration are such that it cannot pass the pyloric valve under the fasted or fed conditions. Generally, the device 1000 may have a shape and/or a size and/or a structural rigidity enabling the device to be retained at the predetermined location. The capability of the device 1000 to be retained may be defined in accordance with methods described in details herein below with reference to FIG. 10A.

In the embodiments wherein the predetermined location is the ICV region, the device 1000 optionally includes a trapping assembly configured for blocking cooperating objects while allowing chyme flow when the device is positioned in the expanded configuration at the ICV. The cooperating objects may be ingested objects e.g., dosage forms. Particularly, the device in the expanded configuration may be configured to block objects having dosage form dimensions. For example, the device in the expanded configuration may be configured to block cylindrical or other commonly shaped tablet dosage forms. Particularly, the device in the expanded configuration may be configured to block spherical beads of a diameter larger than a trapping threshold diameter of e.g., 7 mm, 8 mm, 9 mm, 10 mm, 11 mm or 12 mm. In the following, the predetermined dimensions of the ingested objects may refer to the dimensions of said ingested objects when reaching the ileocecal valve region i.e., after transit through the GI tract. In some embodiments, the device 1000 may be configured so as to allow passage of spherical beads of a diameter lower than a passage threshold diameter of e.g., 3 mm, 4 mm, 5 mm or 6 mm. In some embodiments, the frame of the device is configured to form the trapping assembly.

The device 1000 is further configured to transfer into an emptying configuration in which it can pass through the pyloric valve or ICV under standard GI motility conditions. In some embodiments, the capability of the device 1000 to pass through the pyloric valve or ICV in the emptying configuration may be defined in accordance with methods described in detail herein below with reference to FIG. 10A.

The device 1000 may comprise a body 1010, an opening assembly in the form of a biasing assembly 1030 and a trigger assembly 1040. The device 1000 may comprise at least three (e.g., four) articulated arms 1011-1014 arranged (e.g., longitudinally) alongside each other (e.g., along longitudinal axis X) and forming a body 1010 of the device (see FIG. 1C). An articulated arm is an elongated structure that includes at least one articulation between two sections or segments, allowing bending during which the two sections substantially maintain their shape.

Each articulated arm may be composed of a set of (e.g., two) (e.g., rigid) arm sections. For example, the articulated arm 1011 may be composed of a first arm section 1112 and a second arm section 1114, the articulated arm 1012 may be composed of a first arm section 1122 and a second arm section 1124, the articulated arm 1013 may be composed of a first arm section 1132 and a second arm section 1134, and the articulated arm 1014 may be composed of a first arm section 1142 and a second rigid section 1144.

Each arm section 1112, 1114, 1122, 1124, 1132, 1134, 1142, 1144 may comprise one or more (e.g., one or two) integrally formed components. Each such integrally formed component may be rigid, meaning that it has no or low resilience (in particular, a substantially zero resilience or a resilience significantly lower than the resilience of the resiliently deformable member 1035). Each integrally formed component may be made of any rigid material, such as plastic (e.g., a resin), metal, or ceramic. Each integrally formed component may be 3D printed or (e.g., injection) molded. This facilitates manufacturing. Each integrally formed component may be made of a biocompatible material. The biocompatible material may optionally be biodegradable, and further optionally be configured for being retained at the predetermined location for a predetermined time and for a partial or complete erosion/softening before emptying from the body. For example, each integrally formed component may be made of MEDIC® rigid material manufactured by Stratasys® 3D printer or Visijet M3 Crystal 3D printer by Objet® or for example made of cellulose acetate or other biocompatible relevant polymers manufactured by molding. The rigidity of the articulated arms may provide high structural rigidity and retention capability of the device (capacity of the device to be retained), while achieving a particularly high ratio of expanded volume relative to compactness of the closed configuration, due to allowed thinness of the arms. The arm sections may be made in different materials one from another.

Each articulated arm 1011-1014 may have a first end (e.g., 1117) and a second end (e.g., 1119). The first end 1117 and the second end 1119 of articulated arm 1011 only are given a numerical reference in the figures (see FIG. 1B), for the sake of conciseness. Each first end (e.g., 1117) may be an end of the first arm section (e.g., 1112) of the articulated arm (e.g., 1011), while each second end (e.g., 1119) may be an end of the second arm section (e.g., 1114) of the articulated arm (e.g., 1011).

The device 1000 may comprise a first coupling head 1032 and a second coupling head 1034. The first and second coupling heads 1032 and 1034 may be substantially identical in their external shape, each substantially centered on longitudinal axis X, and/or arranged substantially symmetrically one relative to another with respect to a median plane (not shown) of the device 1000 substantially perpendicular to axis X.

Each coupling head may be integrally formed and/or made of any material (identical to or different from the arm sections), for example a rigid material, such as plastic (e.g., a resin), metal, or ceramic, or a semi-rigid or flexible material. Each coupling head component may be 3D printed or (e.g., injection) molded. This facilitates manufacturing. Each coupling head may be made of a biocompatible material. The biocompatible material may optionally be biodegradable, and further optionally be configured for being retained at the predetermined location for a predetermined time and for a partial or complete erosion/softening before emptying from the body. For example, each coupling head may be made of MEDIC® rigid material manufactured by Stratasys® 3D printer or Visij et M3 Crystal 3D printer by Objet® or for example made of cellulose acetate manufactured by molding. The rigidity of the coupling heads may provide high structural rigidity of the device. In the expanded configuration (see FIGS. 1A-1C), the first end (e.g., 1117) of each articulated arm (e.g., 1011) is coupled to (i.e., connected to, cooperating with) the first coupling head 1032, and the second end (e.g., 1119) of each articulated arm (e.g., 1011) is coupled to the second coupling head 1034. Thus, the first and second coupling heads 1032 and 1034 are configured to join the (e.g., four) articulated arms 1011-1014 to each other by both ends, such that the arms 1011-1014 are secured alongside each other in the expanded configuration.

In the closed configuration (see FIGS. 1D-1F), the first end (e.g., 1117) of each articulated arm (e.g., 1011) may also be coupled to the first coupling head 1032, and the second end (e.g., 1119) of each articulated arm (e.g., 1011) may be coupled to the second coupling head 1034. Thus, the arms 1011-1014 are secured alongside each other in the closed configuration as well.

The first end (e.g., 1117) of each articulated arm (e.g., 1011) may be rotatably coupled to the first coupling head 1032, and the second end (e.g., 1119) of each articulated arm (e.g., 1011) may be rotatably coupled to the second coupling head 1034. In other words, each articulated arm 1011-1014 may be connected to the first coupling head 1032 and/or to the second coupling head 1034 but with freedom in rotation relative thereto.

The respective arm sections 1112 and 1114, 1122 and 1124, 1132 and 1134, and 1142 and 1144 of each articulated arm 1011-1014 may further be coupled end to end with a pivot-type coupling (e.g., 1113). For each articulated arm (e.g., 1011) consisting of two arm sections (e.g., 1112 and 1114), each arm section may be coupled (e.g., rotatably) at one end to the first or second coupling head 1032 or 1034 and coupled (e.g., rotatably) at the other end to the other arm section. For example, the first arm section 1112 of the articulated arm 1011 is coupled (e.g., rotatably) at its one end 1117 (i.e., first end of the articulated arm 1011) to the first coupling head 1032 and at its other end 1167 to the other (i.e., second) arm section 1114 of the articulated arm 1011 with a pivot-type coupling 1113. Similarly, the second arm section 1114 of the articulated arm 1011 is coupled (e.g., rotatably) at its one end 1119 (i.e., second end of the articulated arm 1011) to the second coupling head 1034 and at its other end 1169 to the other (i.e., first) arm section 1112 of the articulated arm 1011 with the pivot-type coupling 1113. The pivot-type coupling 1113 of the articulated arm 1011 only is given a numerical reference on the figures (see FIG. 1B), for the sake of conciseness.

The device 1000 may thus form a (e.g., rigid) mobile (e.g., Sarrus) mechanism deformable from the closed configuration of FIGS. 1D-1F into the expanded configuration of FIGS. 1A-1C, optionally reversibly deformable between from the expanded configuration of FIGS. 1A-1C into the closed configuration of FIGS. 1D-1F, by converting relative rotations into a relative motion (e.g., a translation along axis X) between the first coupling head 1032 and the second coupling head 1034. The converted relative rotations are the relative rotation of each pair of coupled arm sections one with respect to the other, and the relative rotation of each arm section coupled to a coupling head 1032 or 1034 with respect to said head. Such motion conversion achieves bending (e.g., respectively, straightening) of the articulated arms to (e.g., reversibly) deform the device from (e.g., respectively, into) the closed configuration into (e.g., respectively, from) the expanded configuration. Indeed, the articulated arms are bent in the expanded configuration (see FIGS. 1A-1C), whereas the articulated arms are substantially straight in the closed configuration (see FIGS. 1D-1E). In examples, due to their rigidity, the rigid arm sections 1112, 1114, 1122, 1124, 1132, 1134, 1142, 1144 maintain their shape during the whole motion conversion, and thereby each present a substantially identical shape between the expanded configuration and the closed configuration. In other words, no rigid arm section is deformed during its relative rotation with another component.

The articulated arms 1011-1014 may be structurally identical one to another. The device 1000 may present a symmetry of revolution with respect to axis X. The arm sections 1112, 1114, 1122, 1124, 1132, 1134, 1142, 1144 may be arranged in one or more (e.g., two) layers between the coupled heads 1032 and 1034. When each articulated arm consists of two arm sections, the first arm sections 1112, 1122, 1132, 1142 may form a first layer while the second arm sections 1114, 1124, 1134, 1144 may form a second layer. The two layers may be separated by the median plane (not shown) of the device 1000 substantially perpendicular to axis X. In other words, pivot-type couplings (e.g., 1113) between the first arm sections 1112, 1122, 1132, 1142 and the second arm sections 1114, 1124, 1134, 1144 may all lie on said plane. The device 1000 may be symmetrical with respect to said median plane.

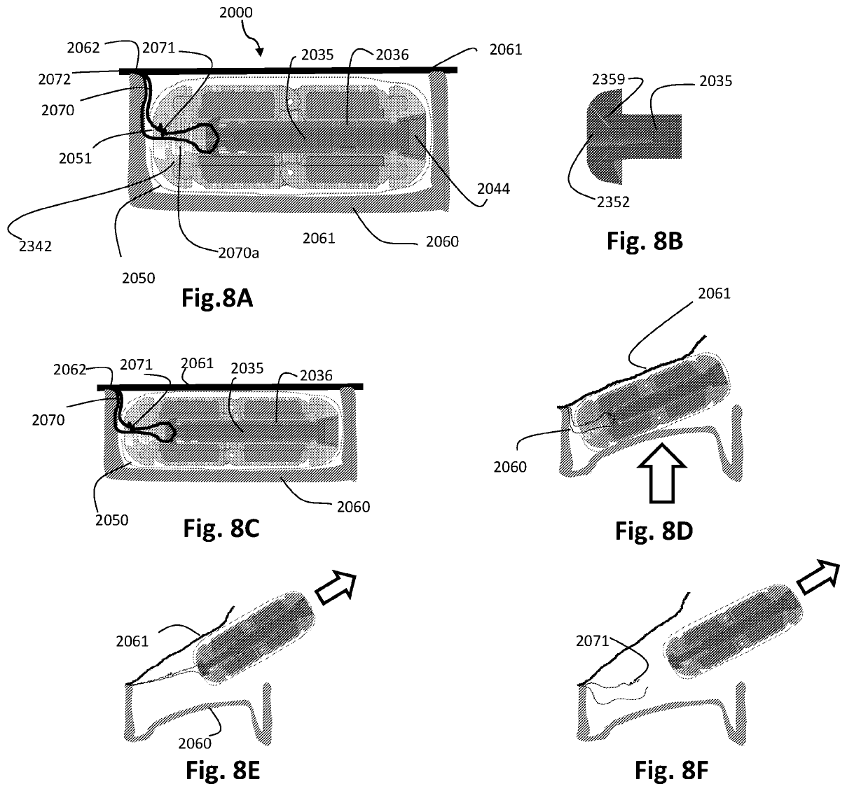

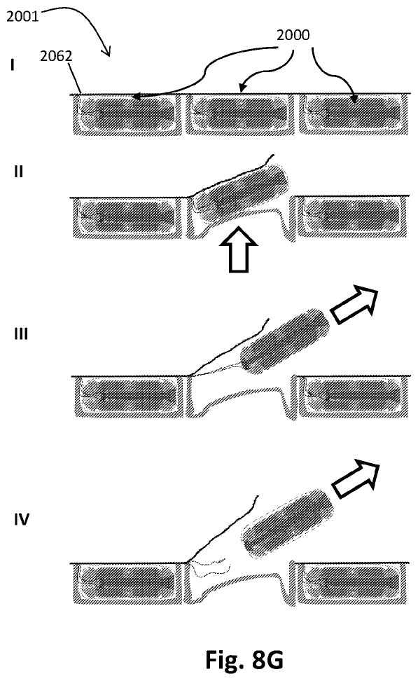

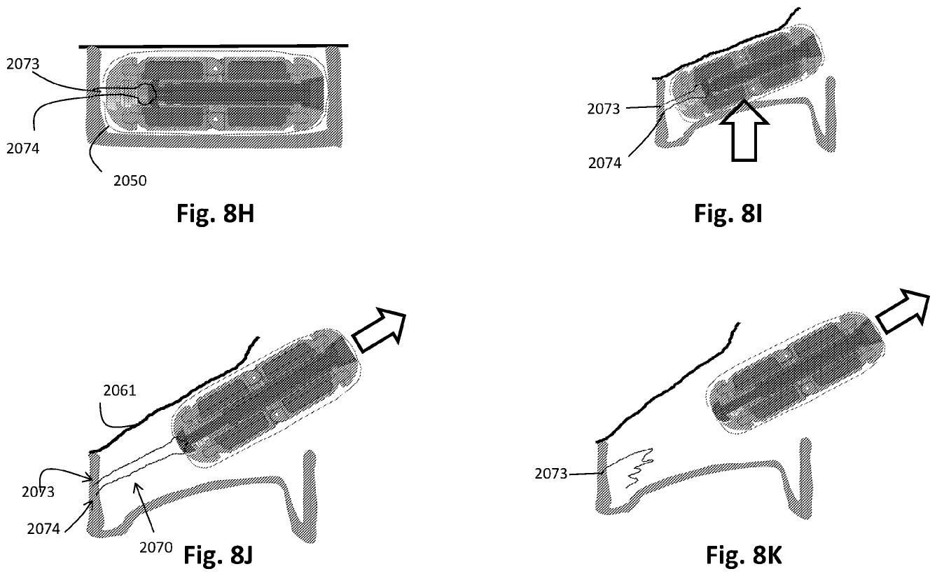

Each arm section 1112, 1114, 1122, 1124, 1132, 1134, 1142, 1144 may be of a generally prism shape (i.e., generally straight). The device 1000 may itself be of a generally prism (e.g., cylindrical) shape in the closed configuration (see FIG. 8D). Each arm section 1112, 1114, 1122, 1124, 1132, 1134, 1142, 1144 may maintain its shape (i.e., not be deformed) during use of the device 1000, for example during transfer from the closed configuration into the expanded configuration, and/or during transfer from the expanded configuration into the emptying configuration.

Each arm section 1112, 1114, 1122, 1124, 1132, 1134, 1142, 1144 may be substantially of a same length, optionally structurally identical. The device may thus have a generally (e.g., regular) bipyramidal shape in the expanded configuration, preferably a generally (e.g., regular) octahedral shape (i.e., when, the number of articulated arms 1011-1014 is exactly four). Each arm section may correspond to an edge of the bipyramidal shape, while each pivot-type coupling (e.g., 1113) and each coupling head 1032 and 1034 may correspond to a vertex of the bipyramidal shape. The device 1000 is thus configured to (e.g., reversibly) switch the generally compact prism/straightened shape of the closed configuration (with a relatively small volume occupancy) into the generally hollow bipyramidal shape of the expanded configuration (with a relatively large volume occupancy). Components of the device 1000 may be rounded at edges and/or vertices of the bipyramidal (e.g., octahedral) shape, such that the device 1000 may present in the (unfolded) expanded configuration a generally spherical convex hull 3D structure.

The rigidity of the articulated arms 1011-1014 allows the device 1000 to present a relatively high structural rigidity while making it easy to maximize use of space. In particular, a 3D structure with a high sphericity (e.g., above 0.8, e.g., measured by the Hakon Wadell sphericity equation of the convex hull of an object) and/or a low ratio between a maximal circumference and a minimal circumference (e.g., below 1.5, e.g., each circumference being the length of a planar curve traced on a periphery of an object) allows a high retention at the predetermined location, while enabling optimal tissue pressure distribution, e.g., when device is in the expanded configuration, and small size when in closed configuration to enable swallowing. This may limit risks of damage to body tissue in any orientation. In examples, the device may maintain a spherical structure, (e.g., as in the examples of the device in the figures), and/or have an expanded configuration with a maximal diameter of less than 25 mm and an enveloped circumference of less than about 80 mm, less than about 75 mm or less than 70 mm. This lowers the risk of pressure on stomach antral tissue or intestinal tissue, in any orientation.

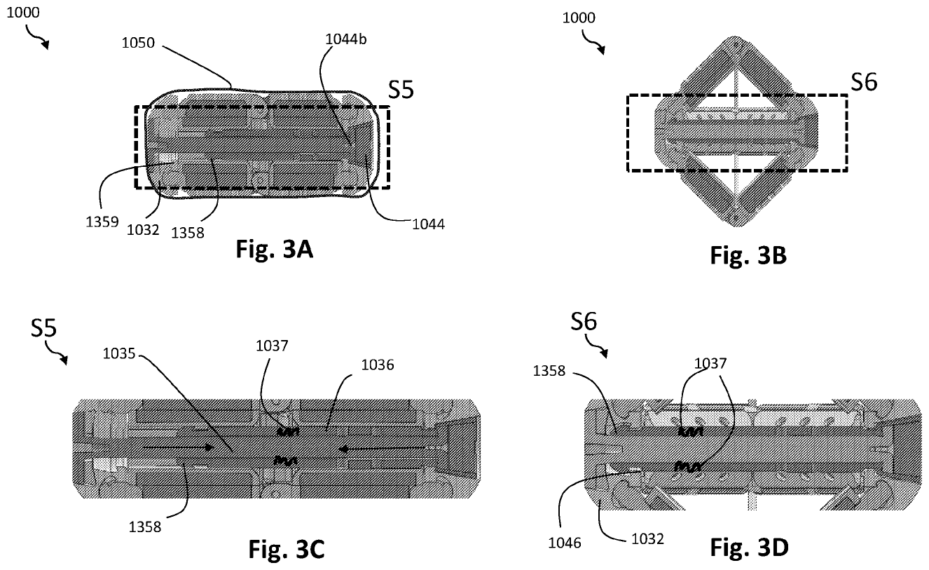

The device 1000 may further comprise a locking element (not shown on these figures, but later shown e.g., on FIG. 3A) arranged to temporary maintain the device in the closed configuration to facilitate oral administration. The locking element may be configured to temporarily overcome the biasing assembly 1030. The locking element may be configured to release the device at the predetermined location into the expanded configuration. In some embodiments, the locking element may be at least partially made of a material dissolving in the environmental conditions of the predetermined location while not dissolving or having a slower dissolving rate in the esophagus so to enable safe swallowing. In some embodiments, the locking element may be a container (e.g., a capsule). The container may be made of or coated for example with Eudragit® E PO that dissolves in pH<5 in the stomach use of the device, or made of an enteric polymer in the ICV use of the device.

In some embodiments, the device 1000 may include a labelling element enabling detection by external detection means. For example, the device may include a barium (or a metal) labelling element which can be detected by detection methods like X-ray imaging.

The device 1000 may operate according to the general operation described below. As explained, the device in the closed configuration may be temporarily maintained in the closed configuration by a capsule as a locking element. Following oral administration, the capsule may dissolve at or before the predetermined location thereby releasing the device into the expanded configuration. Alternatively, the device may be positioned in the expanded configuration at the predetermined location by upper/lower endoscopy. In examples where the predetermined location is the stomach, the device in the expanded configuration moves in the stomach and may at times transit to the pyloric valve region due to GI motility. In examples where the predetermined location is the ICV, following oral administration, the (e.g., enteric coated) capsule may dissolve in the small intestine thereby releasing the device into the expanded configuration. The device in the expanded configuration may transit to the ICV region due to GI motility and position itself at the ICV.

In examples, the volume occupied by the device (e.g., the convex hull) in the expanded configuration may be larger than a sphere of a diameter of 17 mm, and optionally larger than a sphere of a diameter of 20 mm. In examples, the assembly consisting of the articulated arms 1011-1014 and the coupling heads 1032, 1034 in the closed configuration may present dimensions (also referred to as “folded dimensions” or “dimensions of the device when folded” or the like), such that it may be capable of being fitted into a cylinder of less than about 35 mm and of less than about 12 mm diameter. In particular, said assembly in the closed configuration may be capable of being fitted in a cylinder presenting a length of about 30 mm, and/or of diameter equal to or less than 10 mm, less than 28 mm in length and/or less than 9 mm in diameter.

Thus, an optional (e.g., particularly easy to swallow) container (configured to degrade in environmental conditions of the predetermined location, not shown on the figures) configured to temporarily at least partially enclose the device in the closed configuration (e.g., a capsule), may present a length of about 35 mm or less and/or of diameter of about 12 mm or less, for example a length equal to or less than 30 mm and/or a diameter equal to or less than 10 mm, or about 27 mm long and about 8.4 mm diameter.

Such a container may form a locking element and/or be resistant to standard environmental conditions of other parts of the GI tract before arriving at the predetermined location and configured to degrade in environmental conditions of the predetermined location. In variations, the container may merely form a capsule to enable swallowing, and/or the device may comprise another and separate component forming such a locking element arranged to temporary maintain the device in the closed configuration for facilitating administration and/or at least partially made of a material degrading at standard environmental conditions of the predetermined location.

Yet, even with such dimensioning, the device 1000 may present sufficient available space to carry an optional load of an API. The device 1000 may carry a load of an API 1502. The device 1000 may be configured for releasing at least partially the API, e.g., while allowing chyme flow, when the device is positioned at the predetermined location and in the expanded configuration. The load may be solid and/or further contain one or more pharmaceutically acceptable excipients. The solid load may comprise a therapeutic payload of the API for local release and/or topical treatment of a patient condition at the gastric region or for systemic absorption. The API may be any one of the APIs mentioned earlier, and/or for the treatment of any disease or medical condition mentioned earlier. The solid load may comprise no protective cover (e.g., a coating), as the device may comprise a locking container for delivery at the predetermined location, thereby ensuring that the load remains functional (and optionally substantially intact) until it reaches the predetermined location. Alternatively, the solid load may have a coating, so as to release the API only when desired. In variations, the API may be carried by the device in a semi-solid or gel form, or in (any combination of) a solid, semi-solid, powder, gel, and/or liquid form (i.e., partly in one form, and partly in one or more other forms).

Further, even with such dimensioning, the dimensions of the device in the expanded configuration may be such that the device cannot pass through an orifice of about 17 mm diameter and preferably of about 20 mm and achieve high retention. Generally, the device 1000 may have a shape and/or a size and/or a structural rigidity enabling the device to be retained at the predetermined location. The capability of the device 1000 to be retained at the predetermined location may be defined in accordance with methods described in details herein below with reference to FIG. 10A.

In embodiments, each pivot-type coupling (e.g., 1113) may comprise, for each pair of arm sections coupled end to end (by said pivot-type coupling), transversal hinge holes of the arm sections of the pair and a hinge pin passing through the transversal hinge holes. The hinge pin may be made of any rigid material, such as any plastic or any metal or any rigid biocompatible material. The hinge pin may be made, e.g., of nylon or Nitinol. The hinge pin may alternatively be made of an erodible polymer, so as to enable disassembly of the arms upon its erosion to facilitate emptying of the device after retention at the predetermined location. For example, the pivot-type coupling 1113 may comprise a hinge pin 1115 (see FIG. 1B) passing through both transversal hinge holes (no numerical reference) formed on the end 1167 of the first arm section 1112 and transversal hinge holes (no numerical reference) formed on the end 1169 of the second arm section 1114. In examples, the end 1167 of the first arm section 1112 may comprise a first set of one or more (e.g., two) fingers 1126 and the end 1169 of the second arm section 1114 may comprise a second set of one or more (e.g., two) fingers 1128 (see FIGS. 1A and 1C). The fingers 1126 and 1128 may extend longitudinally from their respective arm sections and be dimensioned for the first and second sets of fingers to engage one with another, thereby reaching alignment of all the transversal hinge holes, thus allowing passage of the hinge pin 1115.

The device 1000 may optionally comprise one or more (e.g., one or two) circumferential threads 1015 circumferentially linking the articulated arms (see FIGS. 1C, 1D and 1F). Each thread 1015 may be made of an elastic or a flexible material so as to allow the articulated arms 1011-1014 to open into the expanded configuration without substantial restriction. For example, each thread 1015 may be made of silicone or fabric, synthetic polymer such as nylon, polyurethane or absorbable sutures (e.g., Ethicon). Each thread 1015 may pass through transversal threading holes (no numerical reference) formed on the articulated arms. Each thread 1015 may be pulled through transversal threading holes and tied at the end. The transversal threading holes may comprise one or more (e.g., one or two) layers of transversal threading holes, each layer being on a respective plane perpendicular to axis X. For example, each first arm section 1112, 1122, 1132, 1142 may comprise a respective transversal threading hole forming together a first layer of transversal threading holes perpendicular to the axis X, and each second arm section 1114, 1124, 1134, 1144 may comprise a respective transversal threading hole forming together a second layer of transversal threading holes perpendicular to the axis X (thus parallel to the first layer of transversal threading holes). A same thread 1015 may be pulled through each respective layer of transversal threading holes and tied at the end. Optionally, a same unique thread may pass through the optional different layers, or alternatively one single thread may be pulled per layer. In variations, a thread 1015 may be pulled through the hinge holes of the pivot-type couplings (e.g., 1113) and serve for both circumferentially linking together the articulated arms and as a hinge connector (i.e., for achieving the hinge mechanism of the pivot-type couplings). In other words, the thread serves as both a belt and in place of a hinge pin.

The body 1010 may form the frame of the device 1000. The device 1000 may be configured to bend the articulated arms 1011-1014 in an angular shape (in the expanded configuration). When not bent, the arms 1011-1014 may substantially extend in the direction of the longitudinal axis X.

When the device 1000 is in the open (expanded) configuration, the body 1010 and the one or more (e.g., one or two) circumferential threads 1015 may form a tridimensional meshed structure. The openings 1020 formed between the articulated arms 1011-1014 and the circumferential threads 1015 in the expanded configuration may define a mesh of said meshed structure. The openings 1020 may be configured to enable chyme flow.

The circumferential threads 1015 may have a structural function, for example stabilize the platform formed by the device 1000 in the expanded configuration. This improves the retention capability.

The device 1000 may be configured to transit within the GI tract until arriving at the predetermined location (e.g., by being blocked by the pyloric valve or ileocecal valve) in the expanded configuration without damaging the patient tissue. The device 1000 may have an external envelope configured to contact the patient tissue. In particular, the device 1000 may be flexible to avoid damaging the patient tissue. The external envelope of the device 1000 may be blunt (unsharpened). A maximum diameter of the device in the expanded configuration may be below about 30 mm or below about 25 mm.

The device 1000 in the expanded configuration has an external envelope configured to contact the subject tissue (see FIG. 1A). The external envelope comprises the external surface 1321 of the first coupling head 1032, the external surface 1341 of the second coupling head 1034, and the external surface (e.g., 1162, 1164) of each articulated arm (e.g., 1011). The external surfaces 1162, 1164 of the first and second arm sections 1112, 1114 of articulated arm 1011 only are given a numerical reference on the figures, for the sake of conciseness. The external envelope of the device 1000 may be blunt to avoid damaging the subject tissue. In particular, the coupling heads 1032, 1034 may be made of a smooth material (e.g., plastic, such as resin, or polyurethane, or other biocompatible material) and/or of an externally smooth (e.g., rounded) shape, for example of a general shape of a section of a sphere or ovoid (e.g., semi-sphere or semi-ovoid). Also, the articulated arms 1011-1014 may be made of a smooth material (e.g., plastic, such as resin) and/or of an externally smooth shape, for example of a general shape of a longitudinal section of a cylinder or a prism having an elliptical base. In addition, the external surface (e.g., 1162, 1164) of the ends (e.g., 1167, 1169) of the arm section (e.g., 1112, 1114), for example the external surface of the fingers 1126 and 1128, may be longitudinally rounded. Thus, even when they are bent, the articulated arms 1011 present a smooth vertex at the pivot-type coupling region between the arm sections, to avoid damaging tissue.

The biasing assembly 1030 may be configured to resiliently hold (bias) the device 1000 in the expanded configuration. In other words, the biasing assembly 1030 may be configured to cause the device to tend to resiliently return from the closed configuration into the expanded configuration.

The biasing assembly 1030 may comprise two coupling heads 1032, 1034 and a resiliently deformable (biasing) member 1035. A first end of each flexible arm may be coupled to the first coupling heads 1032 and a second end of each flexible arm may be coupled to the second coupling head 1034. The resiliently deformable member 1035 may be arranged between the articulated arms 1011-1014 and configured to force the coupling heads 1032 and 1034 together to bend the arms thereby biasing the device in the expanded configuration. Thus, the coupling heads 1032 and 1034 come close one to another in the expanded configuration due to retraction of the resiliently deformable member 1035 (i.e., the coupling heads 1032 and 1034 are closer one to another in the expanded configuration than in the closed configuration).

The resiliently deformable member 1035 may be configured to be stretched (along longitudinal axis X, into the closed configuration). For example, the resiliently deformable member 1035 may be elastic (i.e., made of an elastic material, such as silicone). For example, the resiliently deformable member 1035 may be made of silicone shore 55 A, for example as a section of a silicone tube having a cross section area of about 3 mm².

The resiliently deformable member 1035 may be secured to both the first and second coupling heads 1032 and 1034. The resiliently deformable member 1035 may comprise a first extension 1352 and a second extension 1354 at its extremities (i.e., edges), and a central resiliently deformable portion 1353 therebetween (see FIG. 1E). The first extension 1352 may be secured to the first coupling heads 1032, and the second extension 1354 may be secured to the second coupling heads 1034. The first and second extensions 1352, 1354 may or not be unitary with the central resiliently deformable portion 1353. In the latter case, the central resiliently deformable portion 1353 may be secured at its two extremities to the first and second extensions 1352, 1354. The first and second extensions 1352, 1354 may or not be resiliently deformable. The first and second extensions 1352, 1354 may form extension straps.

The first coupling head 1032 may comprise a first longitudinal hollow portion formed along axis X between a first external opening 1322 and a first base opening 1324, and/or the second coupling head 1034 may comprise a second longitudinal hollow portion formed along axis X between a second external opening 1342 and a second base opening 1344 (see FIG. 1B). The resiliently deformable member 1035 may be configured to have the first and second extensions 1352 and 1354 inserted and secured into the first and second hollow portions, for example by the first and second extensions 1352 and 1354 being pulled through the first and second base opening 1324 and 1344 and then secured in the first and second hollow portions.

The resiliently deformable member 1035 may be secured to at least one (e.g., only one, or alternatively, both) of the first and second coupling heads 1032 and 1034 releasably. When the resiliently deformable member 1035 is released from the first coupling head 1032 and/or from the first coupling head 1034, the resiliently deformable member 1035 is prevented from forcing the coupling heads together 1032 and 1034. Thus, the resiliently deformable member 1035 cannot impart bending of the articulated arms 1011-1014. This allows transfer from the expanded configuration into an emptying configuration (by deactivation of the locking member 1036, see FIG. 1E, as later discussed in more details), the device being thereby configured to move past the predetermined location (e.g., pass through the pyloric valve, or the ileocecal valve).

Alternatively or additionally, the device 1000 may carry a load of API (not shown on the figures) free inside an inner space 1022 formed in the closed configuration around the resiliently deformable member 1035 and delimited by the arms 1011-1014 and the coupling heads 1032, 1034 (see FIG. 1E). The API may be carried inside the inner space 1022 in any manner, for example in a solid (e.g., pellets), semi-solid, powder, gel, and/or liquid form. Alternatively or additionally, the device may comprise an exposed recess formed on an internal surface of each arm section (not shown in the figures). The exposed recess may lodge a load of API (not shown), for example filling the recess. The API may be lodged inside the recess in any manner, for example in a solid (e.g., pellets), semi-solid, powder, gel, and/or liquid form.

The arms 1011-1014 may be at least partially made or coated with a material composed at least in part of the API. As the arms 1011-1014 are exposed to chyme flow, the material may diffuse the API as it erodes.

The device 1000 carries a load of API and is retained at the predetermined location (e.g. ICV or stomach), where the API is released over a desired period of time. Optionally, in the embodiments wherein the predetermined location is the ICV region, once positioned at the ICV of the patient, the device described herein may be used in cooperation with a cooperating object in the form of an oral dosage form. An oral dosage form for cooperation with the device may have dimensions so as to enable trapping by the device when reaching the ICV after oral administration. The dosage form (e.g., a tablet) may comprise a therapeutic payload (i.e., an active pharmaceutical ingredient (API)) for local release and/or topical treatment of a patient condition at the ICV region. The dosage form may advantageously comprise a protective cover (e.g., a coating) so as to remain functional (and optionally substantially intact) until it reaches the ICV region. For example, the dosage form may have a protective cover such that less than a predetermined ratio of the API (e.g., less than 10%, 20%, 30%, 40%) is released prior to reaching the ICV. Optionally, once positioned at the ICV of the patient, the device described herein may be used in cooperation with a cooperating object in the form of an oral dosage form. An oral dosage form for cooperation with the device may have dimensions so as to enable trapping by the device when reaching the ICV after oral administration. The dosage form (e.g., a tablet) may comprise a therapeutic payload (i.e., an API) for local release and/or topical treatment of a patient condition at the ICV region. The dosage form may advantageously comprise a protective cover (e.g., a coating) so as to remain functional (and optionally substantially intact) until it reaches the ICV region. For example, the dosage form may have a protective cover such that less than a predetermined ratio of the API (e.g., less than 10%, 20%, 30%, 40%) is released prior to reaching the ICV. Following oral administration, the cooperating dosage form transits along the GI until reaching the ICV region where it is trapped by the trapping assembly of the device. At the ICV, the API is then released from the cooperating dosage form for a predetermined time. The release of the API progressively downsizes the cooperating dosage form until it passes through the trapping assembly of the device. Subsequent cooperating dosage forms can be administered similarly. The device enables frequent administration of a cooperating oral dosage form while lowering the risk of obstruction and enabling tissue relaxation. For example, the cooperating dosage form may be daily administered for a period of 2-8 weeks for example for treating UC. Such option allows combining treatment by the API initially carried by the device with treatment by the API contained in the oral dosage form. Alternatively, the load of API carried by the device may be sufficient, such that no oral dosage form need be administered to the patient.

Following oral administration, in some embodiments, the device may be used in conjunction with an object in the form of an emptying dosage form that is configured to cause transfer of the device into the emptying configuration (i.e., to activate the trigger assembly 1040). In some embodiments, as described above, the trigger assembly may be activated by the device being exposed to a predetermined set of activation environmental conditions. The emptying dosage form may be configured to cause the environmental conditions of the predetermined location to reach the activation environmental conditions. Basically, in these embodiments, an emptying dosage form for use in conjunction with the device may comprise a payload (in the form of an emptying agent) enabling to cause the environmental conditions of the predetermined location to reach the predetermined set of activation environmental conditions. For example, the trigger assembly may be activated when a surrounding pH reaches above respectively below a predetermined threshold (e.g., above pH 6 respectively below pH 4), depending on whether the device is configured to be retained in the stomach respectively within the intestinal tract such as at the ICV, and the emptying dosage form may be configured with a basic or an acidic payload.

Following oral administration, the emptying dosage form transits along the GI until reaching the predetermined location. In case the device is used in the intestine such as at the ICV, the emptying oral dosage form may be trapped by the device. At the predetermined location, the payload of the emptying dosage form is released so as to cause the trigger assembly of the device to activate thereby causing transfer of the device into the emptying configuration.

Alternatively or additionally, the device 1000 may be configured for the trigger assembly 1040 to self-activate after being exposed to the GI tract environment over a predetermined period of time, so as to empty the device 1000 (i.e., without the use of such an emptying dosage form). In embodiments, the device forms relatively little obstruction to chyme flow, such that the device may safely remain retained at the predetermined location until emptying, for example after a period of at least 1.2, 1.5 or 2 times longer that the period of time required to fully diffuse the carried API, or the period for at least a desired amount of the API to be released at the predetermined location of device retention (i.e., 50% of the API released in ICV before emptying of the device).

The device 1000 may comprise a trigger assembly 1040 (see FIG. 1E), forming an opening assembly with the biasing assembly 1030. The trigger assembly 1040 may be configured, when the device in the expanded configuration is exposed to any respective activation signal from a predetermined set of one or more activation signals, to trigger release of the resiliently deformable member 1035 from the first and/or second coupling heads 1032, 1034. The device 1000 may be configured to transfer into an emptying configuration in which it can pass through the pyloric valve or ICV under standard GI motility conditions. In some embodiments, the capability of the device 1000 to pass through the pyloric valve or ICV in the emptying configuration may be defined in accordance with methods described in details herein below with reference to FIG. 10A.

The trigger assembly 1040 may include a first support element 1042 configured for securing the first extension 1352 to the first coupling head 1032, and/or a second support element 1042 configured for securing the second extension 1354 to the second coupling head 1034.

The trigger assembly 1040 comprises two support elements 1042, 1044 configured to temporary maintain the device into the expanded configuration. The support elements 1042, 1044 are configured to be disabled when the device in the expanded configuration is exposed to a predetermined activation signal. The support elements 1042, 1044 are configured to cooperate with the biasing assembly 1030 so that, when the support elements 1042, 1044 are disabled, the biasing assembly 1030 is irreversibly disabled and the device 1000 transfers into the emptying configuration. The trigger assembly 1040 may be configured to cause additional degrading of the device such as for example, disassembling into two or more subcomponents, additional decline of shape, size and/or structural rigidity.

As can be seen, the emptying configuration can resemble the closed configuration. In some embodiments, the emptying configuration can even be identical to the closed configuration.

In some embodiments, the support elements 1042, 1044 may have a pin shape. The first and second extensions 1352, 1354 may for example be shaped as socks or comprises portions shaped as socks in which the support elements 1042, 1044 can be inserted via the first and second external openings 1322, 1342. The pin-shaped support elements 1042, 1044 may be inserted in the first and second extensions 1352, 1354 while press-fitted inside the first and second hollow portions (of the first and second coupling heads 1032, 1034). The press-fitting may maintain the first and second extensions 1352, 1354 securely attached to the first and second coupling heads 1032, 1034. Alternatively or additionally, the first and second hollow portions of the first and second coupling heads 1032, 1034 may comprise locking corners 1325, 1345, that cooperate with an extremal portion of the pins-shaped support elements 1042, 1044, to locally increase the press-fitting force and improve retention of the resiliently deformable member 1035 by the support elements 1042, 1044.

In some embodiments, the support elements 1042, 1044 may be erodible elements configured to degrade when the device is exposed to an activation signal, and the erosion of the support elements releases the coupling heads 1032, 1034 from the resiliently deformable member 1035 so that the device is irreversibly allowed to go into the emptying configuration in which it may be capable of being emptied through the pyloric valve or ICV. The support elements 1042, 1044 may be erodible layer-by-layer starting from the first and second external openings 1322, 1342, which are exposed to the environment of the device 1000, and going toward the first and second base openings 1324, 1344. As long as there remains at least a layer of a support element 1042 or 1044 with a diameter higher than a diameter of the first or second base opening 1324 or 1344, the support element 1042 or 1044 maintains its supporting function. As soon as the erosion reaches a (triggering) point where no such layer remains in the support element 1042 and/or 1044, the resiliently deformable member 1035 is free to retract and detach from the respective coupling head 1032 and/or 1034. Such a triggering point may be said to activate the trigger assembly 1040, and each support element 1042, 1044 may be referred to as a “timer”.

Thanks to the first and second external openings 1322, 1342 being formed on top of the coupling heads 1032, 1034 (i.e., opposite to the base of the coupling heads 1032, 1034 where the articulated arms are coupled), the first and second external openings 1322, 1342 are oriented longitudinally (on axis X), with no element of the device 1000 forming an obstruction. This arrangement allows a particularly fine control of the support element or timers 1042, 1044, thereby, when at least one of the timers 1042, 1044 is triggered, the resiliently deformable member 1035 may shrink backwards, thus releasing extension 1354 from the corresponding head openings 1322 or 1342 and the device unfolds.

Pin-shaped support elements 1042, 1044 may be made of any material and/or manufactured as described in reference to the device 1000. An activation signal increasing or decreasing the pH at the predetermined location may be used to erode the pin to disassembly the device

In some examples, when the predetermined location is the stomach, pin-shaped support elements 1042, 1044 may be made of Eudragit® L or S or HPMCAS LG, LM, HG grades and may be manufactured as follows: a Hot Melt Extrusion (HME) machine is set to for example about 130° C. to 160° C. Material powder is fed into the HME machine for example by a gravimetric feeder at a rate of 1 kg/hr. The HME machine snail speed is set to 100 rpm. The melted material is drawn from the HME machine, it is forwarded as strands onto a conveyor belt to cool. Once cooled, the strand is chopped by a chopping machine to a pin shape of about 1.5 mm diameter and 2 mm length.

In some examples, when the predetermined location is the ICV region, pin-shaped support elements 1042, 1044 may be made of Eudragit® E may be manufactured as follows: a Hot Melt Extrusion machine is set to for example about 150° C. Eudragit® E powder is fed into the HME machine for example by a gravimetric feeder at a rate of 1 kg/hr. The HME machine snail speed is set to 100 rpm. The melted material is drawn from the HME machine, it is forwarded as strands onto a conveyor belt to cool. Once cooled, the strand is chopped by a chopping machine to a pin shape of about 1.5 mm diameter and 2 mm length. For example, the support elements 1042, 1044 may be configured to be disabled after the device 1000 is exposed to standard environmental conditions of the predetermined location for a predetermined residence time period, for example of 12 hr, one day, two days, three days or more and/or twelve weeks or less (e.g., any time period from 1 to 12 weeks, such as one month, one week, two weeks, three weeks, or even one day or two days). In other words, the trigger assembly 1030 may activate after the device is positioned at the predetermined location for a predetermined time period or after a predetermined time period elapsed subsequent to swallowing of the device. In some embodiments, support elements configured to be disabled after the device is exposed to standard environmental conditions for a predetermined residence time period may be made of a combination of a material degrading at standard environmental conditions of the predetermined location (such as Eudragit® E PO; copolymer based on dimethylaminoethyl methacrylate, butyl methacrylate, and methyl methacrylate) and a control release delay material (such as a polymer carrier). The support elements 1042, 1044 may also or alternatively be configured to be disabled after the device is exposed to a predetermined set of activation environmental conditions. For example, the support elements 1042, 1044 may be configured to be disabled when a surrounding environment reaches (e.g., above or below) a predetermined pH threshold (e.g., pH 5, 6, 7 or 8 or below pH 6, 5, or 4). For this purpose, the support elements 1042, 1044 may be erodible elements at least partially made of a material configured to dissolve (or degrade) when the surrounding pH reaches the predetermined pH threshold. In some embodiments the support elements 1042, 1044 may be at least partially made of a material dissolving at (i.e., when the pH reaches) the predetermined pH threshold. For example, the support elements may be made of a base-soluble polymer or an acid-soluble polymer, depending on the predetermined location. For example, in the case of retention in the stomach, the support elements 1042, 1044 may be at least partially made of a material soluble at of a material soluble at pH above 6 such as Eudragit® L or of a material soluble at pH above 7 such as Eudragit® S. In other examples, in the case of retention at the ICV, the support elements 42, 44 may be at least partially made of a material soluble at pH below 5 such as Eudragit® E PO.

In some examples, when the predetermined location is the stomach, the support elements 1042, 1044 may be at least partially made of a material soluble at pH above 6 such as enteric polymer such as Eudragit® L or for example HPMCAS LG, or of a material soluble at pH above 7 such as Eudragit® S or for example HPMCAS HG). In some embodiments, the support elements 1042, 1044 may have a pin shape. Pin-shaped support elements 1042, 1044 of Eudragit® L or S may be manufactured as follows: a Hot Melt Extrusion machine is set to for example about 120-140° C. HPMCAS LG powder is fed into the HME machine for example by a gravimetric feeder at a rate of 1 kg/hr. The HME machine snail speed is set to 100 rpm. The melted material is drawn from the HME machine, it is forwarded as strands onto a conveyor belt to cool. Once cooled, the strand is chopped by a chopping machine to a pin shape of about 1.5 mm diameter and 2 mm length. In some embodiments, the support elements 1042, 1044 may be at least partially made of a material dissolving in standard stomach environmental conditions, such as Eudragit®, and coated with a material dissolving at the predetermined pH threshold, such as Eudragit® L or S. Such pin-shaped coated support elements may be manufactured as follows: The Hot melt extrusion machine (HME) is preheated to for example about 120-150° C. The HME machine snail speed is set to 100 rpm. Powder of Eudragit® E PO is added into HME feeder and pushed by the snail outwards. As the melted material is drawn from the HME machine, it is forwarded as strands onto a cooling machine to cool on a conveyor belt. Once cooled, the strand is chopped by a chopping machine to pin shape of about 1.5 mm diameter and 2 mm length. The pins are then placed in a coating system (e.g., vector coater) and coated with Eudragit® L or S coating with a total of for example about 10% of weight gain. In some embodiments, at least one of the support elements 1042, 44 is configured to be disabled after the device is exposed to standard environmental conditions at the predetermined location for a predetermined time period, and at least one of the support elements 1042, 1044 is configured to be disabled after the device is exposed to a predetermined set of activation environmental conditions, for example a surrounding pH reaching above pH 6.

In some examples, when the predetermined location is the ICV region, the support elements 1042, 1044 may be at least partially made of a material dissolving in standard ICV region environmental conditions, such as hydroxypropylmethylcellulose acetate succinate (HPMC-AS), and coated with a material dissolving at the predetermined pH threshold, such as Eudragit® E PO. Such pin-shaped coated support elements may be manufactured as follows: Powder of HPMC-AS is premixed with Dibutyl sebacate (DBS) in ratio of 8:1 in DIOSNA mixer for 5 minutes at 500 rpm. After premixing, the mixture is placed at room temperature for 24 hr so that the polymer and plasticizer settle together. After 24 hr, the mixture is fed into the HME machine. The HME machine is preheated to for example about 150° C. The HME machine snail speed is set to 100 rpm. As the melted material is drawn from the HME machine, it is forwarded as strands onto a cooling machine to cool on a conveyor belt. Once cooled, the strand is chopped by a chopping machine to pin shape of about 1.5 mm diameter and 2 mm length. The pins are then placed in a coating system (e.g., vector coater) and coated with Eudragit® E PO coating with a total of for example about 10% of weight gain. In some embodiments, at least one of the support elements 1042, 1044 is configured to be disabled after the device is exposed to standard ICV environmental conditions for a predetermined time period and at least one of the support elements 1042, 1044 is configured to be disabled after the device is exposed to a predetermined set of activation environmental conditions, for example a surrounding pH reaching below pH 5.

The support elements 1042, 1044 may be arranged to form keystones of the device in the expanded configuration so as to provoke a collapse of the device into the emptying configuration after the device is exposed to the predetermined activation signal. In other words, the support elements 1042, 1044 are arranged to form one or more structural weak points. When the support elements 1042, 1044 are disabled, the one or more structural weak points cause the device to fall apart in the emptying configuration. The support elements 1042, 1044 are arranged so that a transfer duration of the device from the expanded configuration into the emptying configuration is substantially smaller (e.g., equal or less than ½, ⅓, ¼, ⅕, ⅙, 1/7, ⅛, 1/9, 1/10, 1/100, 1/1000) than a standard residence time period (e.g., equal or more than 1, 2, 3, 4, 5, 6, 7 or 8 weeks). This provides an improved control of the device in in-vivo conditions. In particular, in embodiments in which the trigger assembly is activated by external means, this enables to quickly empty the device on-demand without requiring an endoscopy procedure even in urgent cases.

In embodiments, the support elements 1042, 1044 may be configured to be disabled upon the device in the expanded configuration being exposed to a different activation signal. This provides flexibility in the manner of transferring the device 1000 into the emptying configuration. For example, the support elements 1042, 1044 may be made of different materials. For example, the support element 1042 may be made of a type A material, while the support element 1044 may be made of a type B material. Utilizing two timers constructed of different materials provides a safety mechanism in the event that the timer intended to dissolve first does not function properly, or in the event that the device unexpectedly and prematurely transitions into a second compartment, such as from the stomach into the intestine while the platform is in an expanded configuration. In this case, the first timer designed to dissolve in the stomach will not function in the intestine but the second “safety timer” which is designed to dissolve in the small intestine will be activated and the platform will transform into the emptying configuration.

The device 1000 may be configured for the disassembling of the body 1010, formed by the articulated arms, from the first and second coupling heads 1032, 1034 upon activation of the trigger assembly 1040 (see FIG. 1G). Such exploding of the device 1000 from the expanded configuration into the emptying configuration facilitates the emptying when the trigger assembly 1040 activates, thus increasing safety of use. FIG. 1G shows an emptying configuration where not only the body 1010 is disassembled from both the first and second coupling heads 1032, 1034, but also the resiliently deformable member 1035 is disassembled from both the first and second coupling heads 1032, 1034. This is because both support elements 1042, 1044 have been degraded to an activation point. In embodiments, the device 1000 may achieve one or more other emptying configurations. For example, the resiliently deformable member 1035 may remain attached to one of the coupling heads 1032, 1034 and detach only from the other one, when only one of the support elements 1042, 1044 has been degraded to the activation point. This is likely to occur when different support elements/timers 1042, 1044 configured to degrade under different activations signals are used. Still, the body 1010 is disassembled from both the first and second coupling heads 1032, and this may be enough for a secure emptying.

In reference to FIGS. 1H-1K, the disassembling of the body 1010 from both the first and second coupling heads 1032 is now discussed in more details.

FIGS. 1H-1J show the device 1000 respectively in the closed, expanded, and emptying configuration, corresponding to a standard sequence of use of the device 1000. FIG. 1K further illustrates the sequence of use by showing close-up views of a zone S1-S4 of the device where the first arm section 1112 of the articulated arm 1011 is coupled to the first coupling head 1032.

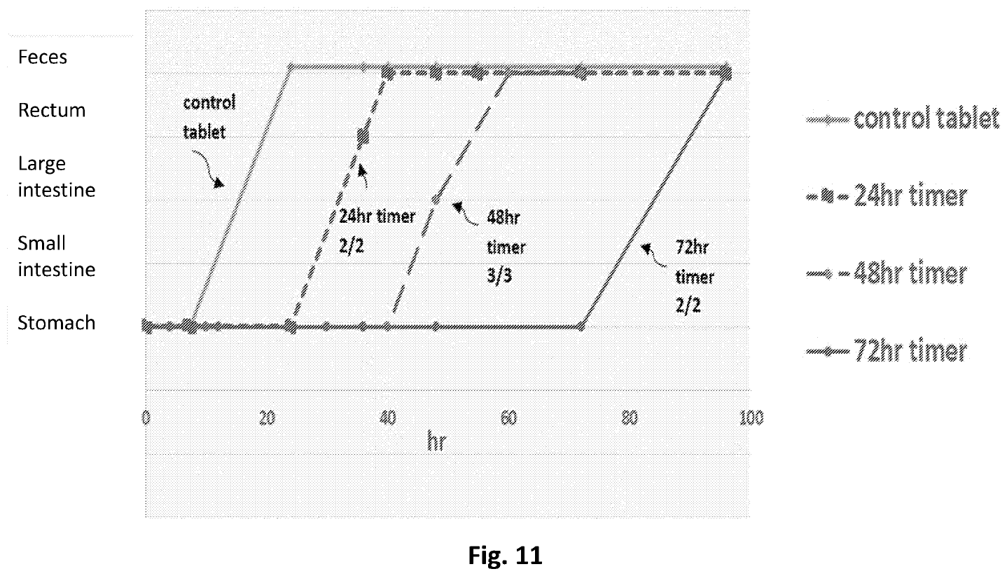

As shown, the first end (e.g., 1117) of each articulated arm (e.g., 1011) is coupled to the first coupling head 1032 releasably, and the second end (e.g., 1119) of each articulated arm (e.g., 1011) is coupled to the second coupling head 1034 releasably. In the expanded configuration (see FIG. 11), said first end 1117 is inserted in a respective cavity 1326 of the first coupling head 1032 (see FIG. 1K, last step, i.e., close-up S4), and said second end 1119 is inserted in a respective cavity of the second coupling head 1034 (not shown). Indeed, the resiliently deformable member 1035 maintains said first end and second end 1117 and 1119 each secured inside the respective cavities. This is thanks to the resiliently deformable member 1035 being securely attached to the first and second coupling heads 1032-1034 by the support elements 1042, 1044, thus pulling the first and second coupling heads 1032-1034 together when transferring from the closed configuration (FIG. 111) into the expanded configuration (FIG. 11). The retraction of the resiliently deformable member 1035 and the movement of the first and second coupling heads 1032-1034 one toward the other is represented by arrows on FIGS. 111-1K.

The respective cavity 1326 of the first coupling head 1032 may be dedicated to the coupling of the articulated arm 1011, and each other articulated arm 1012-1014 may cooperate with a different other cavity of the first coupling head 1032. Such arm head cooperation increases the stability of the assembled device when in open unfolded configuration while exposed to external forces (e.g., when a force is applied on arm couplings, e.g., 1113) Similarly, the second coupling head 1034 may comprise a single cavity per articulated arm 1011-1014. Alternatively, any or both the first and second coupling heads 1032 and 1034 may comprise a circumferential cavity (e.g., 1326) configured to receive a respective end of each articulated arm 1011-1014.

Referring to FIG. 1K in particular, the discussion now focuses on the coupling and release of the first arm section 1112 of the articulated arm 1011 with respect to the first coupling head 1032. But the discussed coupling and release may identically apply to the first arm section of each other articulated arm 1012-1014 with respect to the first coupling head 1032, and/or to the second arm section (e.g., 1114) of each articulated arm 1011-1014 with respect to the second coupling head 1034.

The first end 1117 (and thus the first arm section 1112) may be rotatable in the respective cavity 1326. Thus, when the device 1000 transfers from the closed configuration (close-up S1) into the expanded configuration (close-up S2), the first end 1117 makes a rotation R1 relative to the first coupling head 1032, due the resiliently deformable member 1035 pulling the coupling heads 1032 and 1034 together. Similarly, when the device 1000 transfers from the expanded configuration (close-up S2) into the emptying configuration, the first end 1117 initially makes an inverse rotation R2 relative to the first coupling head 1032 (close-up S3), due to the locking member 1036 and/or resiliently deformable member 1035 being detached and thus stopping maintaining the expanded configuration, thus allowing the GI mechanical forces to apply on the device 1000 (e.g., due to chyme flow and tissue-induced pressures), thereby straightening the articulated arm 1011. Afterwards, when the device continues to transfer from the expanded configuration into the emptying configuration, the first end 1117 may be dimensioned to move out of the respective cavity 1326, due again to the GI mechanical forces. Thus, the first arm section 1112 and the first coupling head 1032 form a hinge disassembly mechanism.

As best seen on close-up S2, the first arm section 1112 may transit in an angle to the first coupling head 1032 and then be locked in head. The first coupling head 1032 may comprise for that a stopper mechanism. The stopper mechanism may comprise dents 1320 of the first coupling head 1032 cooperating with recesses 1110 of the first end 1117 (see close-up S4). Surfaces forming the recesses 1110 may engage surfaces forming the dents 1320, thereby preventing disassembly of the arm from the coupling head in the expanded configuration, even if GI mechanical forces increase.

In embodiments, in the folded configuration, the arm and head are orientated in a same axis while the arm is forced forward into the head cavity by the resiliently deformable to member. During unfolding, the arm transits in an opposite angle to the head, and this positions the arm in a locking position within the head. After the timer is released, the resiliently deformable member is detached, the arm returns in a backward movement back into the folding position. As the resiliently deformable member is now detached, the arm is no longer forced into the head, and thus can be disassembled from the head when a mild external force is applied.

In the device 1000, at least one (e.g., all eight) of the arm sections 1112, 1114, 1122, 1124, 1132, 1134, 1142, 1144 comprises an exposed cavity (no numerical reference on the figures).

The cavity allows embedding any desired product inside the arm section. As the cavity is exposed, the embedded product is in fluid communication with the surrounding environment of the device 1000, notably when the device 1000 is in the expanded configuration. In examples, rigid arm sections may easily comprise such cavities thanks to their rigidity. In specific, because of said rigidity, the presence of the cavities does not prevent from having a sufficient structural rigidity of the device 1000, such that it better remains functional and open in the expanded configuration so as to be retained at the predetermined location.

In particular, the cavity may contain an API. The API may be provided within a solid form 1502 such as a tablet, which may optionally further contain one or more pharmaceutically acceptable excipients. The solid form 1502 may comprise a therapeutic payload of the API for local release and/or topical treatment of a patient condition at the gastric region or for systemic absorption. The API may be any one of the APIs mentioned earlier, and/or for the treatment of any disease or medical condition mentioned earlier. The solid form 1502 may comprise no protective cover (e.g., a coating), as the device 1000 may comprise a locking container (e.g., capsule) for delivery at the predetermined location, thereby ensuring that the solid form 1502 remains functional (and optionally substantially intact) until it reaches the predetermined location. Alternatively, the solid 1502 may have a coating and/or the exposure apertures 1508, 1510, 1512 (discussed hereinbelow) in the cavity may be coated, so as to expose the inner solid form 1502 only when desired (i.e., delay mechanism). In variations, the API may be carried by the device in a semi-solid, powder, gel, or liquid form/texture, or in (any combination of) a solid, semi-solid, powder, gel, and/or liquid form (i.e., partly in one form, and partly in one or more other forms).

At least one (e.g., all eight) of the arm sections 1112, 1114, 1122, 1124, 1132, 1134, 1142, 1144 may comprise two components (e.g., recipient or body component 1504 and a cover component 1506) attached one to another and forming the exposed cavity therebetween. Numerical references are provided only for the arm section 1112 and its two components 1504 and 1506 (see FIG. 1G), for the sake of conciseness. Each such component (e.g., 1504 and 1506) may be rigid, and optionally integrally formed, for example made by injection molding or 3D printing or any other known way, from any material such as plastic, a biocompatible polymer such as cellulose acetate, resin, metal, or ceramic, as discussed with reference to the device 1000. The two components of each pair may be attached in any manner, for example snapped (i.e., clicked) one to another, e.g., after insertion of the solid form (e.g., tablet) 1502 between the two components. This facilitates manufacturing. The at least one arm section (e.g., 1114) may comprise a peripheral wall. Each component of the at least one arm section (e.g., 1114) may comprise one or more respective walls, and said respective walls may compose the peripheral wall. The peripheral wall may have apertures 1508, 1510, 1512 (i.e., openings or holes) formed thereon, providing exposure of the cavity and thereby drug release at the predetermined location (see FIGS. 1B and 1C). The apertures 1508, 1510, 1512 connect the cavity with the environment of the device, thus putting the cavity into fluid communication with said environment.

One or more (e.g., all) recipient components (e.g., 1504) may comprise such drug release apertures 1508, and/or one or more (e.g., all) cover components (e.g., 1506) may comprise such drug release apertures 1510, 1512. This provides for drug release on one or both sides of each such arm section, thereby achieving a uniform diffusion of the solid form 1502. Alternatively, the apertures may present a design which provides a predetermined release rate.

As shown for the arm section 1114 (see FIGS. 1B and 1C), the recipient component may comprise one or more (e.g., exactly one) ranges of drug release apertures 1508, formed on an external envelope of the device 1000. Each range of drug release apertures 1508 may be linear, straight, and/or evenly distributed. Alternatively or additionally, the cover component may comprise one or more (e.g., exactly two) ranges of drug release apertures 1510, 1512. Each range of drug release apertures 1510 and/or 1512 may be linear, straight, and/or evenly distributed. Each arm section 1114 may present a generally prism shape with a triangle or trapeze cross-section. The drug release apertures 1508 may be formed on a face of the prism corresponding to the base of the triangle or trapeze (i.e., respective wall of the recipient component), and the drug release apertures 1510, 1512 may be formed each on a respective face of the prism to the sides of the triangle or trapeze (i.e., respective walls of the cover component). This achieves an even more uniform diffusion of the solid form 1502, by ensuring exposure everywhere of the cavities inside the arm sections.

The arm sections 1112, 1114, 1122, 1124, 1132, 1134, 1142, 1144 may be dimensioned to encounter each other when the device 1000 reaches the expanded configuration (as best seen on FIG. 1C), thus maintaining a minimal distance between the two coupling heads 1032 and 1034, thereby preventing the device 1000 from expanding beyond the maximal authorized diameter. For example, the arm sections (e.g., 1132, 1134) of each articulated arm (e.g., 1013) may comprise each a sloped surface (e.g., 1532, 1534), and each such pair of sloped surfaces may be configured to abut one onto another, so as to maximize the use of space (see FIG. 1G). In view of the presence of the cavities (e.g., already embedding an API), the device 1000 may have a relatively reduced volume occupancy in the expanded configuration (while allowing sufficient drug loading), thus allowing space occupancy induced by said abutting sloped surfaces (e.g., 1532, 1534).

The device 1000 may comprise a unique thread 1015 located in a single plane. The thread 1015 may lie on a median plane (not shown) of the device 1000 substantially perpendicular to axis X. The hinge pins 1115 part of the pivot-type coupling between the arm sections may be hollow, and the thread may pass through said hinge pins 1115. In some variations, the device 1000 may comprise several threads (e.g., in several planes) and/or a unique thread in several planes. Alternatively, the thread 1015 may be pulled through the hinge holes of the pivot-coupling hinge before the hinge pins are inserted, or threads may be glued to the hinge pins. Alternatively, the thread can serve as both a circumferential belt and a hinge pin. The thread may be composed of two sections, including a more flexible sections and a more rigid and less flexible section (optionally also wider, designed to be retained by pressure in the hinges cavities). The thread may be pulled through the hinge hole while the rigid section may be placed and retained into the arm hinge as a pin.

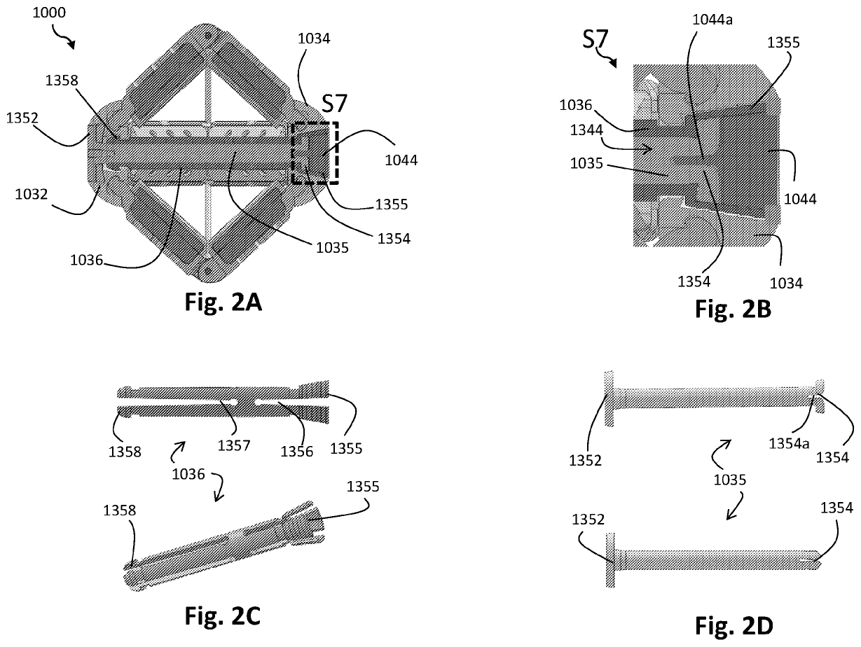

Referring to FIGS. 2A-2D, the device 1000 may further comprise a locking assembly part of or cooperating with the biasing assembly 1030 and configured to, when activated, maintain the device 1000 in the expanded configuration. The locking assembly may comprise a locking member 1036 arranged between the two coupling heads 1032 and 1034 and having two ends 1355 and 1358. The locking member 1036 includes one anchor-shaped end 1358 cooperating with a respective coupling head 1032 and one timer housing end 1355 cooperating with a respective coupling head 1034 via a support element 1044. On FIG. 2A, the device 1000 is in the expanded configuration and the anchor-shaped end 1358 is attached to coupling head 1032. The locking member 1036 is connected at the other end 1355 to the other coupling head 1034 via a support element 1044 (see FIG. 2B). The support element 1044 may support both the end 1354 of the resiliently deformable member 1035 and the end 1355 of the locking member 1036, and attach both of them to the coupling head 1034 (see close-up S7 in FIG. 2B).

As can be seen on FIG. 2C, the locking member 1036 presents a longitudinal shape and may be of a generally tubular shape.

The locking member 1036 may comprise longitudinal grooves 1356 that provide deformability to the end 1355. Thereby, the diameter of said end 1355 may be reduced or increased. This enables insertion and retention of the end 1355 by coupling head 1034 in the expanded configuration, thanks to conical shape of support element 1044 that peripherally presses the end 1355 against the wall of the receiving recess of coupling head 1034. This also enables reduction of the diameter of the end 1355 when the support element 1044 is degraded, so as to enable release of the locking member 1036, by its end 1355 exiting coupling head 1034 by base opening 1344.

The locking member 1036 may further comprise longitudinal grooves 1357 that provide deformability to the anchor-shaped end 1358. Thereby, the diameter of said anchor-shaped end 1358 may be reduced or increased. This facilitates snapping of the anchor-shaped end 1358 in coupling head 1032.

As can be seen on FIG. 2D, resiliently deformable member 1035 presents a longitudinal shape and may be of a generally tubular shape, insertable inside the locking member 1036. The outer diameter of resiliently deformable member 1035 may be lower or substantially equal to the inner diameter of the locking member 1036. Extension 1354 may be stretchable so as to be straightened (see bottom of FIG. 2D) and inserted inside locking member 1036 from the opening at anchor-shaped end 1358, and then retrieve a skirt shape presenting a slit 1354a once out of the locking member 1036 (see top of FIG. 2D). In the example of FIGS. 2A-2B, support element 1044 comprises an extension 1044a that enters slit 1354a and press-fits the extensions 1354 against the end 1355 of the locking member 1036, so as to attach both in coupling head 1034. In variations shown on FIGS. 1A-1K, the support element 1044 comprises no such extension and retains the end of the resiliently deformable member merely by pressing on the extensions 1354 thereof (for the sake of concision, the same numerical references are used for the device 1000 and the support element 1044 on FIGS. 1A-1K and FIGS. 2A-2D, although they slightly differ by the presence or not of such extension 1044a). In other variations, a screw (not shown on the figures) may be inserted through support element 1044 in slit 1354a.

FIGS. 3A-3B as well as the close-ups S5-S6 in FIGS. 3C-3D illustrate activation of the locking member 1036. In the closed configuration (FIG. 3A and its close-up S5 in FIG. 3C), the anchor-shaped end 1358 is free from coupling head 1032. Thus, the locking assembly is inactive. The resiliently deformable member 1035 is configured to unfold the device 1000 by retraction of the resiliently deformable member 1035, represented by arrows in FIG. 3C. Yet, such unfolding (i.e., transferring the device into the expanded configuration) is prevented by capsule container 1050. As shown on FIG. 3B and its close-up S6 in FIG. 3D, transferring of the device from the closed configuration of FIG. 3A into the expanded configuration activates the locking assembly by snap-fitting. Snap-fitting the locking assembly is achieved by snapping the free anchor-shaped end 1358 of the locking member 1036 into a recess of the respective coupling head 1032. The anchor shape then cooperates with an edge or locking tooth 1046 that retains the locking member, thus preventing folding back of the device.

The locking member 1036 is configured to engage the free end 1358 to respective coupling head 1032 head when the device transfers from the closed configuration into the expanded configuration, thereby attaching (i.e., fixedly connecting) the end 1358 to its respective coupling head 1032. As a result, the locking member 1036 biases/maintains the assembly in the expanded configuration, by maintaining the two coupling heads 1032,1034 at a fixed distance. Thus, the locking member 1036 “shortens” the resiliently deformable member 1035, which is thereby put at no or lesser use once the locking assembly is activated.

The locking member 1036 may be made of a rigid or semi-rigid material, at least more rigid than the resiliently deformable member 1035. Thus, the locking member 1036 allows a secure retention by maintaining the device in the expanded configuration. In examples, the locking member 1036 may be made of a more rigid and/or less elastic material than the resiliently deformable member 1035. The locking member 1036 may notably present a higher tensile strength, a higher tensile modulus, and/or a lower elongation percentage at break, compared to the resiliently deformable member 1035. For example, the locking member 1036 may be made of a rigid or semi-rigid material, such as cellulose acetate, Polyether ether ketone (PEEK), or polyurethane. Additionally or alternatively, the locking member may be shock-absorbing. The locking member 1036 may comprise one or more shock absorbing elements 1037 arranged longitudinally. This allows the device to absorb shocks that it may endure due to GI forces when retained at the predetermined location, even if the locking member is essentially rigid.

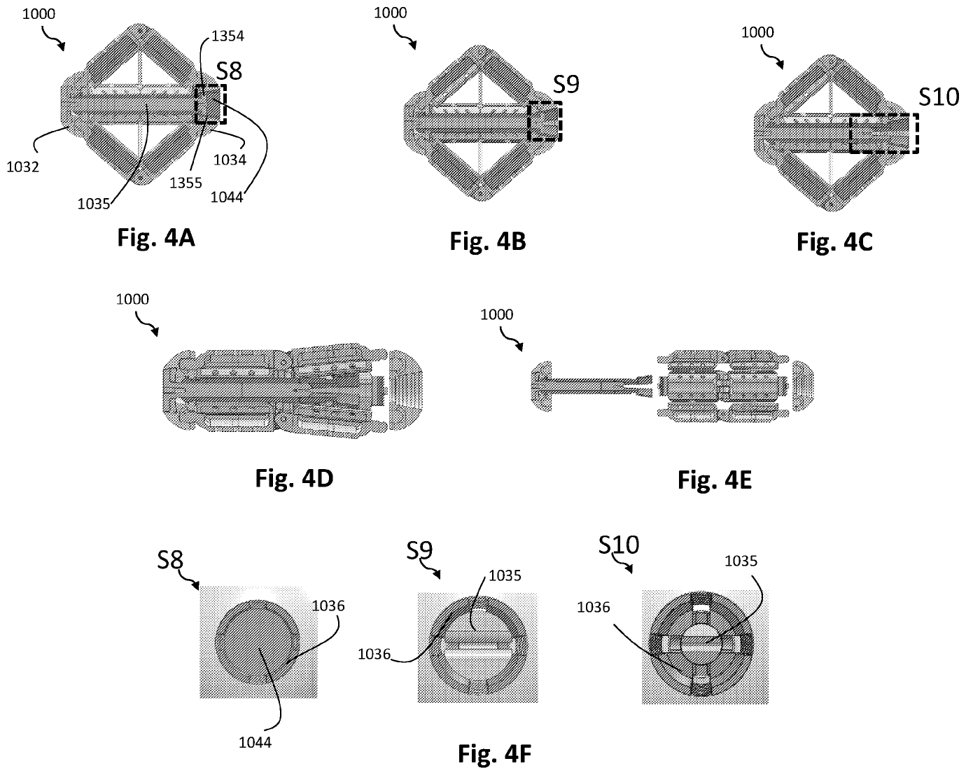

FIGS. 4A-4E illustrate the transferring of the device 1000 from the expanded configuration into the emptying configuration. The device 1000 of FIG. 4A (and its close-up S8 in FIG. 4F) comprises the support element (timer) 1044 which attaches the end 1354 of the (stretched) resiliently deformable member 1035 to the timer housing end 1355 of the locking member 1036 and to the coupling head 1034. In FIG. 4B the support element 1044 is eroded thereby the triggering assembly 1040 is activated and the resiliently deformable member 1035 is no longer attached to the timer housing end 1355 of the locking member 1036 and the coupling head 1034 as the (the skirt of the) extension 1354 is no longer pressed by support element 1044 against the timer housing end 1355 of the locking member 1036, see close-up S9 in FIG. 4F. As a result, the extension 1354 of the resiliently deformable member 1035 is stretched so as to be straightened thereby enabling its transferring to the relaxed state, see FIG. 4C and the close-up S10 in FIG. 4F. Furthermore, upon the activation of the triggering assembly 1040 by the erosion of the support element 1044, the timer housing end 1355 of the locking member 1036 is no longer (peripherally) pressed against the wall of the receiving recess of coupling head 1034, thereby, said end 1355 is no longer retained by the coupling head 1034. Thus, the diameter of the end 1355 is reduced and the locking member 1036 is released as the end 1355 exits coupling head 1034 by base opening 1344, see FIG. 4D. Since the retention force is no longer applied by the biasing assembly 1030 (i.e., neither of the resiliently deformable member 1035 and the locking member 1036) on the coupling heads 1032 and 1034, the device 1000 can be transferred to the emptying configuration by being disassembled, as presented in FIG. 4E.

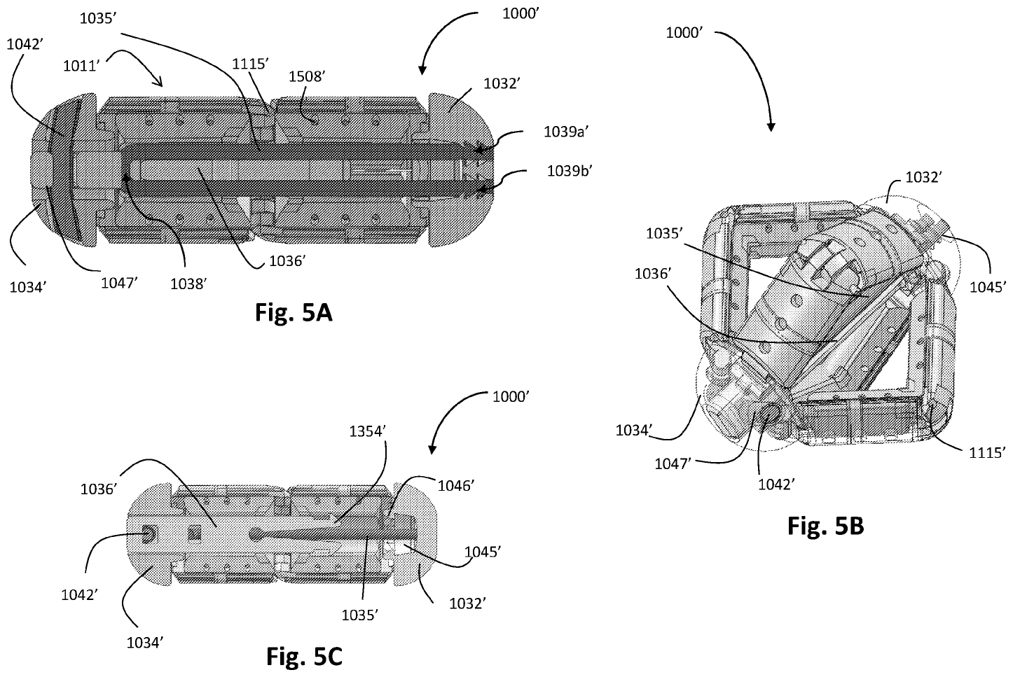

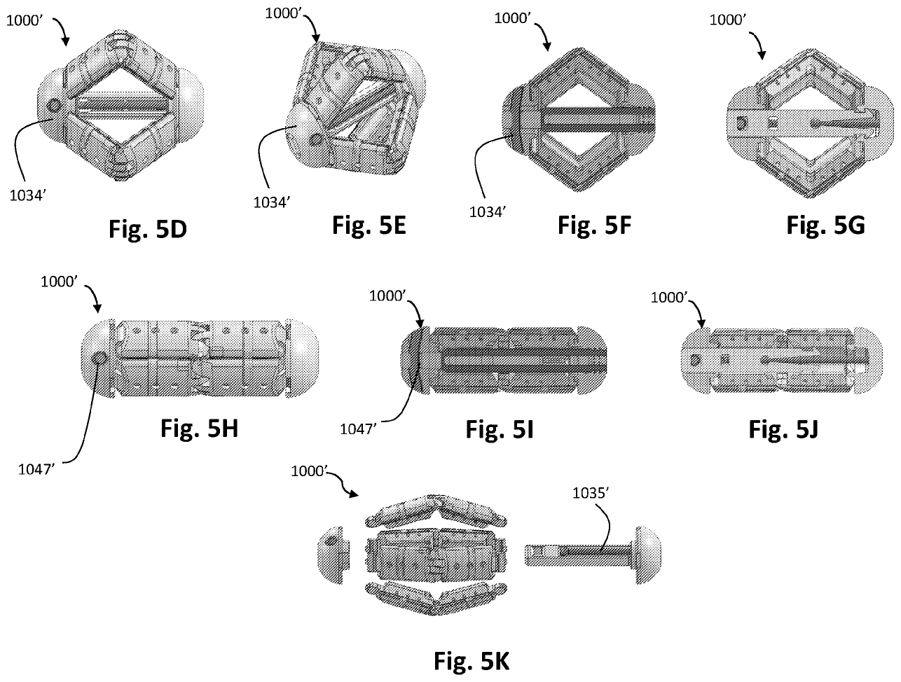

FIGS. 5A-5C, FIGS. 5D-5G, FIGS. 51I-5J and FIG. 5K show another example of a device 1000′ for temporary GI tract retention according to the present disclosure. In particular, they show the device 1000′ in an expanded (open) configuration (FIGS. 5B, 5D-5G), in a closed (swallowing) configuration (FIGS. 5C, 5H-5J), and in a disassembled configuration which may form an emptying configuration and/or a pre-assembling configuration during manufacturing (FIG. 5K). FIGS. 5A, 5C, 5D, and 5F-5J show a front view of the device 1000′. FIGS. 5B and 5E show a perspective view of the device 1000′ in the same configuration as in FIG. 5A.

The device 1000′ resembles the device 1000 discussed above in many aspects.

Accordingly, numerals used to identify features of the device 1000 are appended by a single quotation mark ‘ to identify like features of the device 1000’. It is understood that the features which are similar to device 1000 and combinable with device 1000 are not all repeated in details below.

The device 1000′ is based on the principles of device 1000. In particular, device 1000′ comprises a resiliently deformable member 1035′ and a locking member 1036′. The resiliently deformable member 1035′ is different from 1035 in that the resiliently deformable member 1035′ at least partially covers the outer surface of the locking member 1036′. Locking member 1036′ may have an anchoring orifice 1038′ located between the two ends of the locking member 1036′ through which the resiliently deformable member 1035′ can be pulled anchored to the locking member 1036′. To assemble the resiliently deformable member 1035′ and the locking member 1036′, the resiliently deformable member 1035′ is first pulled through a first opening 1039a′ in head 1032′, then through anchoring orifice 1038′ and then through second opening 1039b′ in head 1032′ (FIG. 5A) to fully anchor the resiliently deformable member in 3 points.

Further the device 1000′ comprises only one supporting element 1042′ (e.g., timer) in the coupling head 1034′. The support element 1042′ may be similar to any of 1042 and 1044 in FIG. 1. Further, the locking member 1036′ may comprise an (integral) extension 1047′ at an end opposite an anchor shape end 1354′, where the extension 1047′ covers the outer surface of the support element 1042′ inside the coupling head 1034′. In some embodiments, the extension 1047′ may have an orifice that can house support element 1042′ when placed inside the coupling head 1034′. An anchor shape end 1354′ (e.g., snap) of the locking member 1036′ is free from the respective coupling head 1334′ in the closed configuration. The anchor shape end 1354′ can be attached (e.g., snap-fitted) to the coupling head 1032′ via a locking tooth 1046′, by moving in the tunnel 1045′ as the device 1000′ is transferred into the expanded configuration. Locking member 1036′ may have an integral hinge in the middle that enables anchor shape end 1354′ to pass over and be locked to locking tooth 1046′.

Further, the device 1000′ may be placed in storage sub-configuration comprising a straining assembly based on principles described for device 2000 (in FIG. 8A). The resiliently deformable member 1035′ of device 1000′ may be attached to a blister (as described in device 2000 in FIG. 8A) by a thread. The resiliently deformable member can be configured to be stretched by the pulling of the thread. Such pulling can be realized naturally during the extraction of the capsule that occupies the device from the blister. Such a thread may have one or more weak points which are configured to be cut (i.e. broken) while the capsule is being pulled out of the blister.

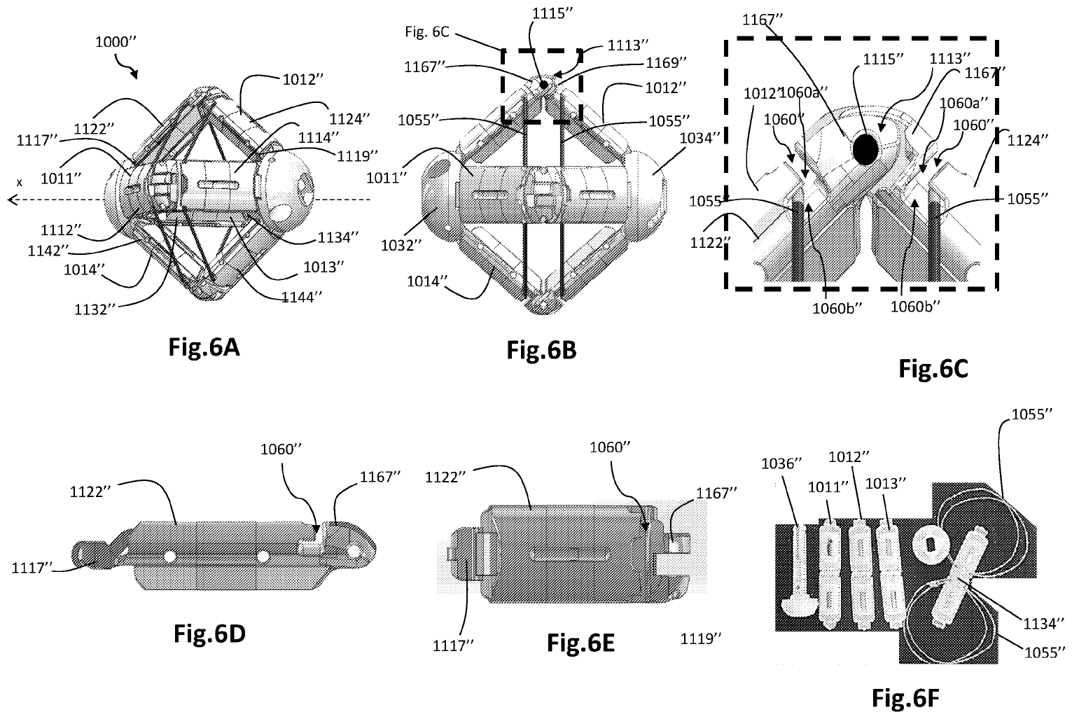

FIGS. 6A-6F show another example of a device 1000″ for temporary GI tract retention according to the present disclosure. In particular, they show the device 1000″ in an expanded (open) configuration (FIGS. 6A-6C) and in a disassembled configuration (FIG. 6F) which may form an emptying configuration and/or a pre-assembling configuration during manufacturing. FIG. 6B shows a front view of the device 1000″, and FIG. 6C shows an enlarged portion of FIG. 6B. FIG. 6A shows a perspective view of the device 1000″ in the same configuration as in FIG. 6B, while FIGS. 6D and 6E show side and top views of a portion of the device 1000″.

The device 1000″ resembles the devices 1000, 1000′ discussed above in many aspects. Accordingly, numerals used to identify features of the devices 1000, 1000′ are appended by two quotation marks “to identify like features of the devices 1000, 1000′. It is understood that the features similar to one or both of devices 1000, 1000′ and combinable with devices 1000, 1000′ are not all repeated in details below.

The device 1000” is based on the principles of device 1000. In particular, device 1000″ comprises a resiliently deformable member (not shown) and a locking member 1036″. The device 1000″ may comprise at least three (e.g., four) articulated arms 1011″-1014″ collectively forming a body 1010″ of the device 1000″. An articulated arm is an elongated structure that includes at least one articulation between two sections or segments, allowing bending during which the two sections substantially maintain their shape.

Each articulated arm may be composed of a set of (e.g., two) (e.g., rigid) arm sections. For example, the articulated arm 1011″ may be composed of a first arm section 1112″ and a second arm section 1114″, the articulated arm 1012″ may be composed of a first arm section 1122″ and a second arm section 1124″, the articulated arm 1013″ may be composed of a first arm section 1132″ and a second arm section 1134″, and the articulated arm 1014″ may be composed of a first arm section 1142″ and a second rigid section 1144″.

Each articulated arm 1011″-1014″ may have a first end 1117″ and a second end (e.g., 1119″). The first end 1117″ and the second end 1119″ of articulated arm 1012″ only are given a numerical reference in the figures (see FIG. 6A), for the sake of conciseness. Each first end (e.g., 1117″) may be an end of the first arm section (e.g., 1112) of the articulated arm (e.g., 1012″), while each second end (e.g., 1119″) may be an end of the second arm section (e.g., 1114″) of the articulated arm (e.g., 1012″).

The device 1000″ may comprise a first coupling head 1032″ and a second coupling head 1034″. The first and second coupling heads 1032″ and 1034″ may be substantially identical in their external shape, each substantially centered on longitudinal axis X, and/or arranged substantially symmetrically one relative to another with respect to a median plane (not shown) of the device 1000″ substantially perpendicular to axis X.

In the expanded configuration (see FIGS. 6A-6C), the first end (e.g., 1117″) of each articulated arm (e.g., 1012″) is coupled to (i.e., connected to, cooperating with) the first coupling head 1032″, and the second end (e.g., 1119″) of each articulated arm (e.g., 1012″) is coupled to the second coupling head 1034″. Thus, the first and second coupling heads 1032″ and 1034″ are configured to join the (e.g., four) articulated arms 1011″-1014″ to each other by both ends, such that the arms 1011″-1014″ are secured alongside each other in the expanded configuration.

The first end (e.g., 1117″) of each articulated arm (e.g., 1012″) may be rotatably coupled to the first coupling head 1032″, and the second end (e.g., 1119″) of each articulated arm (e.g., 1012″) may be rotatably coupled to the second coupling head 1034″. In other words, each articulated arm 1011″-1014″ may be connected to the first coupling head 1032″ and/or to the second coupling head 1034″″ but with freedom in rotation relative thereto.

The respective arm sections 1112″ and 1114″, 1122″ and 1124″, 1132″ and 1134″, and 1142″ and 1144″ of each articulated arm 1011″-1014″ may further be coupled end to end with a pivot-type coupling (e.g., 1113″). For each articulated arm (e.g., 1012″) consisting of two arm sections (e.g., 1122″ and 1124″), each arm section may be coupled (e.g., rotatably) at one end to the first or second coupling head 1032″ or 1034″ and coupled (e.g., rotatably) at the other end to the other arm section. For example, the first arm section 1122″ of the articulated arm 1012″ is coupled (e.g., rotatably) at its one end 1117″ (i.e., first end of the articulated arm 1012″) to the first coupling head 1032″ and at its other end 1167″ to the other (i.e., second) arm section 1124″ of the articulated arm 1012″ with a pivot-type coupling 1113″. Similarly, the second arm section 1124″ of the articulated arm 1012″ is coupled (e.g., rotatably) at its one end 1119″ (i.e., second end of the articulated arm 1012″) to the second coupling head 1034″ and at its other end 1169″ to the other (i.e., first) arm section 1122″ of the articulated arm 1012″ with the pivot-type coupling 1113″. The pivot-type coupling 1113″ of the articulated arm 1012″ only is given a numerical reference on the figures (see FIG. 6B), for the sake of conciseness. In embodiments, each pivot-type coupling (e.g., 1113″) may comprise, for each pair of arm sections coupled end to end (by said pivot-type coupling), transversal hinge holes of the arm sections of the pair and a hinge pin 1115″ passing through the transversal hinge holes. However, other types of hinged connections are also contemplated.

The device 1000″ also can include one or more flexible belts 1055″. In the depicted embodiment, the device 1000″ includes two belts 1055″, though it is contemplated that the device can include more or less belts, i.e., one, three, four, or five or more belts. Each belt 1055″ can be comprised of a polypropylene material, such as PROLENE®. However, it is contemplated that the belt could be comprised of other materials. Each belt 1055″ can comprise a single strand of materials, or alternatively several integrally joined strands of material. Each belt 1055″ can be engaged with each of the articulated arms 1011″-1014″. To secure each belt 1055″ to the device 1000″, each belt 1055″ can be received in channels 1060″ defined by each of the articulated arms 1011″-1014″. In the depicted embodiment, the first and second sections of each articulated arm (e.g., first section 1122″ and second section 1124″ of articulated arm 1112″) each define a respective channel 1060″ for receiving a belt 1055″, where the channel 1060″ of each of the first section 1112″, 1122″, 1132″, 1142″ is equidistantly spaced from the coupling 1113″ of that respective arm 1011″, 1012″, 1013″, 1014″ as the channel 1060″ of each of the corresponding second section 1114″, 1124″, 1134″, 1144″. However, alternative spacing in other embodiments is also contemplated. In operation, the respective channels 1060″ of the first sections 1112″, 1122″, 1132″, 1142″ can be circumferentially aligned with each other so as to receive a first belt 1055″, while the respective channels 1060″ of the second sections 1114″, 1124″, 1134″, 1144″ can also be circumferentially aligned with each other so as to receive a second belt 1055″. When the device 1000″ opens to the expanded configuration, each belt 1055″ can be expanded and stretched, which allows for increased pressure dispersion across the device 1000″, as well as serve to increase the overall footprint of the device 1000″ to improve gastric retention. When the device 1000″ transitions from the expanded configuration to the closed configuration, each belt 1055″ can contract and released from the channels 1060″ within which it was retained, as will be described below.

Referring to FIG. 6C, each channel 1060″ defines a first portion 1060a″ extending inwards from the arm outer surface, and a second portion 1060b″ extending substantially perpendicular to the first portion 1060a″ such that the belt 1055″ can reside within and be retained by the second portion 1060b″ during operation. In addition to the structure of each channel 1060″, each belt 1055″ can be adhered within the channels via an adhesive. The adhesives can be formulated so as to dissolve at a predetermined pH, such as the pH associated with a particular location within the GI tract, such that each belt 1055″ can be released from the channels 1060″ within which it is received during transitioning of the device 1000″ from the expanded configuration to the closed configuration (as shown in FIG. 6F). In some embodiments, the adhesive dissolves in the gastric milieu. In other embodiments, the adhesive dissolves in the intestinal milieu. In yet other embodiments, the adhesive is Eudragit® E PO, for example about 6% in acetone. The adhesive may also be a polyvinylpyrrolidone (PVP, polyvidone or povidone), a cellulose ether (e.g. Klucel®, hydroxypropylcellulose (HPC)) or other known binders.

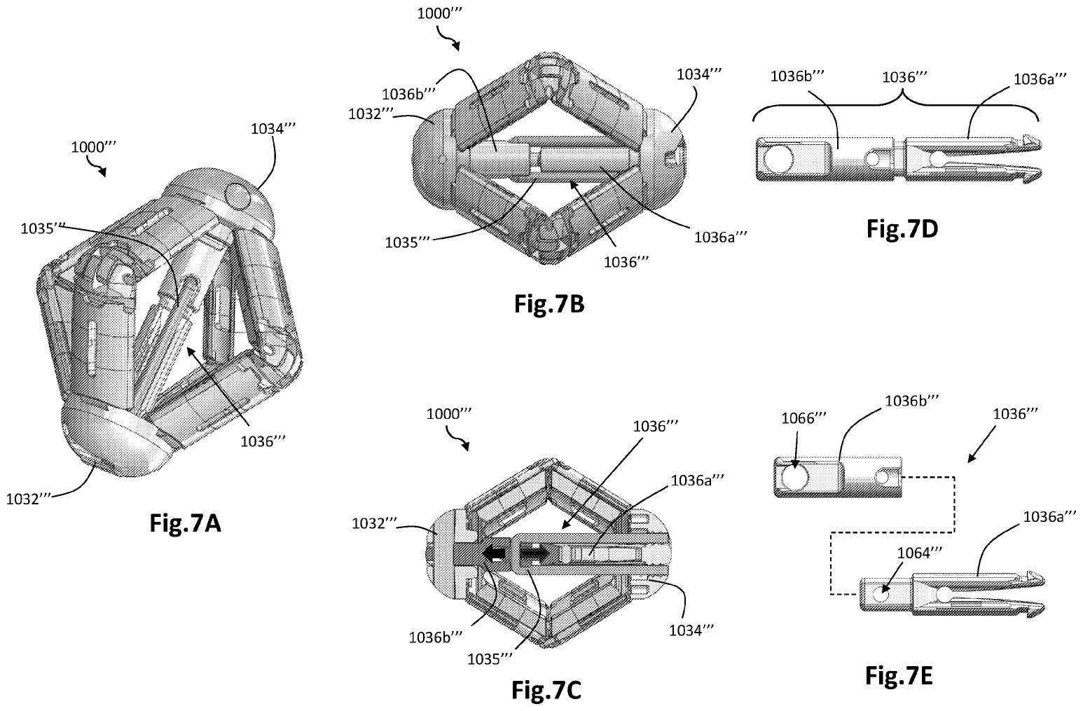

FIGS. 7A-7E show another example of a device 1000′″ for temporary GI tract retention according to the present disclosure. In particular, they show the device 1000′″ in an expanded (open) configuration (FIGS. 7A-7C), along with isolated components of the locking member 1036′″ (FIGS. 7D-7E), which will be described in more detail below. FIGS. 7A and 7B show perspective views of the device 1000′″, while FIG. 7C shows a cross-sectional view of the device 1000′″.

The device 1000′″ resembles the devices 1000, 1000′, 1000″ discussed above in many aspects. Accordingly, numerals used to identify features of the device 1000′″ are appended by three quotation marks ′″ to identify like features of the device 1000′″. It is understood that the features which are similar to devices 1000, 1000′, 1000″ and combinable with devices 1000, 1000′, 1000″ are not all repeated in detail below.

The device 1000′″ is based on the principles of device 1000. In particular, device 1000′″ comprises a resiliently deformable member 1035′″ and a locking member 1036′. The locking member 1036′ is different from locking member 1036 in that the locking member 1036′ has a two-piece construction, i.e., comprises a first portion 1036a′″ and a second portion 1036b′″. The first and second portions 1036a′″, 1036b′″ are axially moveable relative to each other such that the locking member 1036′ acts as a damping member, thus absorbing forces imparted on the device 1000′″ by the GI tract. This increases the durability of the device 1000′″, while reduces the potential risk of damage to the surrounding tissue within the GI tract. In particular, the first portion 1036a′″ may be configured to at least partially received by the second portion 1036b′″. In the depicted embodiment, the first portion 1036a′″ is coupled to the second coupling head 1034′″, while the second portion 1036b′ is coupled to the first coupling head 1032′, though it is contemplated that the inverse arrangement could be implemented.

The first and second portions 1036a′″, 1036b′″ of the locking member 1036′″ can be coupled together by the resiliently deformable member 1035′″. Specifically, the first portion 1036a′″ of the locking member 1036′ can include an aperture 1064′ extending therethrough, while the second portion 1036b′″ of the locking member 1036′″ can include an aperture 1066′″ extending therethrough. The resiliently deformable member 1035′″ can extend through each of the apertures 1064′″, 1066′″ to couple the first portion 1036a′″ to the second portion 1036b′″.

FIGS. 8A-8K show another example of a device 2000 for temporary GI tract retention according to the present disclosure. In particular, they show the front view of the device 2000 in the storage sub-configuration (FIGS. 8A, 8C, 8H), in the dosing sub-configuration (FIGS. 8F, 8K), and the transfer between the storage sub-configuration and the dosing sub-configuration.

The device 2000 resembles the device 1000 discussed above in many aspects.

Accordingly, numerals used to identify features of the device 1000 are incremented by a factor 1000 to identify like features of the device 2000. It is understood that the features which are similar to device 1000 and combinable with device 1000 are not all repeated in details below.

The device 2000 comprises a resiliently deformable member 2035, and a locking member 2036. The locking member 2036 is optional and a same functionality of transferring between the storage sub-configuration and the dosing sub-configuration may be achieved only by a resiliently deformable member 2035. The device 2000 comprises a unique timer 2044; however in variations the device 2000 may comprise another timer 2042 in coupling head 2342 as discussed for the device 1000.

The resiliently deformable member 2035 is kept unstrained during shelf-life (See FIG. 8A) for example when the device is stored in locking capsule container 2050 in a packaging 2060 (e.g., blister). The resiliently deformable member 2035 is strained to bias the device 2000 only before dosing. The straining system resiliently deformable member re-activation is done during conventional extraction of the capsule from the packaging, i.e., by tearing the blister cover 2061 and pressing the container 2050 out of blister 2060. The cover 2061 may be strongly fixed to the main body of blister 2060 at 2062 (e.g., by welding) such that the pressing of the container 2050 from below only (or mainly) tears the cover from the designated side. The arrows in FIGS. 8D-8F, 8G, and 8I-8K represent the forces applied by the end-user. The straining of the resiliently deformable member is done during conventional extraction of the capsule from the blister, such that the end user does not change his/her ordinary dosing sequence.

The device may comprise a straining assembly. The lead 2352 (see FIG. 8B) of the resiliently deformable member 2035 of the device 2000 is attached to the blister 2060 by a thread 2070 which passes through a hatch 2051 on the container 2050 and a threading tunnel 2359 of the resiliently deformable member 2035. The resiliently deformable member 2035 is configured to be stretched by pulling the thread 2070 end via the threading tunnel 2359. Such pulling is realized naturally during the extraction of the capsule 2050 from the blister 2060

In examples according to FIGS. 8A-8G, the thread 2070 may be strongly fixed to the blister at 2062 (e.g., by welding) and to the lead 2352 by passing through the threading tunnel 2359. The thread 2070 may have one or more weak points 2071 (see FIGS. 8A-8F) which are configured to be cut (i.e., broken) while the container 2050 is being pulled out of blister 2060 and after the lead 2352 of the resiliently deformable member 2035 is attached to its respective coupling head 2342 and the lead continues to be pulled. After being cut from the weak point 2071 the thread 2070 is detached from the threading tunnel 2359 and the container 2050 is freed. The lead may stay connected to the blister by a strong connection 2072 which is connected to the blister at the welding point of the blister 2060 to the upper cover 2061.

FIG. 8G shows a sequence of use I-IV in line with the above with an assembly 2001 comprising a packaging 2000 (e.g., blister pack) and devices 2000 inside the packaging (e.g., one device per blister).

In reference to FIG. 811-8K, another example is discussed. The thread 2070 may be attached to the blister 2060 at two points 2073, 2074, and the thread 2070 may be is configured to be detached from at least one of them while the lead is being pulled.

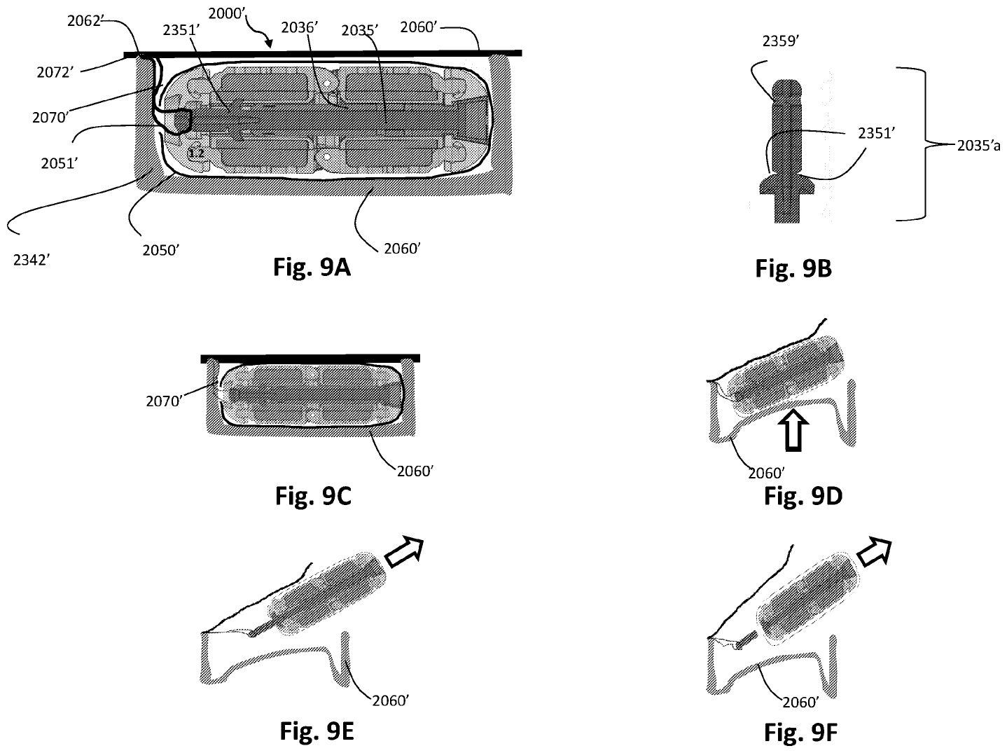

FIGS. 9A-9F show another example of a device 2000′ for temporary GI tract retention according to the present disclosure.

The device 2000 resembles the device 2000′ discussed above in many aspects.

Accordingly, numerals used to identify features of the device 2000 are incremented by a factor 1000 to identify like features of the device 2000′. It is understood that the features which are similar to device 2000 and combinable with device 2000′ are not all repeated in details below.

In this example, the free end of the resiliently deformable member 2035′ comprises an extension 2035′a configured for protruding out of its respective coupling head 2342′ after said end is attached to its respective coupling head, see FIG. 9E. The resiliently deformable member 2035′ comprises a cutting zone 2351′ at the basis of the extension for cutting the extension after said end is attached to its respective coupling head and the lead 2070′ continues to be pulled.

In some variations, a device according to the present disclosure (e.g., device 1000, 1000′, 2000, or 2000′) may comprise no circumferential thread. The device without circumferential thread may allow particularly non-obstructed chyme flow, thus allowing long but safe retention of the device at the predetermined location, thereby being configured for self-emptying from the predetermined location after a predetermined period of time in safety. Yet, the device, according to this variation may be sufficiently rigid even without any circumferential thread or belt.

In some variations, a device according to the present disclosure (e.g., device 1000, 1000′, or 2000) the resiliently deformable member may a spring configured to be stretched (e.g., traction spring), for example made of metal (such as stainless steel or Nitinol (nickel-titanium alloy)). A metal spring has little creep (low compression set) and at the same time a relatively high tension modulus (spring constant). This may allow particularly long shelf-life.

In variations, a device according to the present disclosure (e.g., device 1000, 1000′, 2000, or 2000′) may comprise a support tube. The resiliently deformable member (and optionally the locking member) may be arranged inside the support tube. In such options, the inner space for carrying a load of API may comprise an interstice formed between the support tube and the resiliently deformable member (and optionally the locking member). The support tube may be a hollow tube made of any material, such as a rigid material (e.g., plastic, metal, ceramic) or an elastic material.

In variations where the device comprises a support tube and carries a load of API, the support tube may notably (further optionally) carry a load of an API inside or thereon. When the device expands from the closed configuration into the expanded configuration, the load is exposed to chyme flow through the openings and thereby releases the API. Further, the support tube may have one or more peripheral groove recesses. Each peripheral groove may lodge the API, for example a ring-shaped (e.g., solid) form containing the API. In examples of such options, the support tube may have apertures (on its peripheral wall) which provide exposure (i.e., fluid communication with the physiological environment surrounding the device) to the cavity. This allows fine control of the API release. In particular, the apertures may present a design which provides a predetermined release rate. Alternatively or additionally, the exposure apertures may be coated, so as to expose the contained API load only when desired, or on the contrary uncoated. Alternatively or additionally, the exposure apertures may be coated so as to expose the contained API load only when desired, for example when the device reaches at the predetermined location. Alternatively, the exposure apertures may be uncoated. Alternatively or additionally; the device may include drug, placed in the arms. Apertures may be designed in both arm body/recipient and the arm cover to allow drug release from arms.

In some variations, a device according to the present disclosure (e.g., device 1000, 1000′, 2000, or 2000′) may optionally comprise a padding assembly. The optional padding assembly may comprise a plurality of pads. The pads may be disposed around the device. The pads may be made of a resilient material so as to be reversibly compressible. The pads may be shaped as circles and/or as hollow cylinders. For example, the pads may be made of silicone. Further, the padding assembly may be configured for lowering pressure on the patient tissue when the device is in the open configuration.

In some variations, each arm section of a device according to the present disclosure (e.g., device 1000, 1000′, 2000, or 2000′) may be semi-rigid or may include flexible material (e.g., silicone) thereby enabling flexibility upon expanding of the device. In yet other variations, each arm section may comprise any combination of one or more rigid components, one or more semi-rigid components, and/or one or more flexible components. The device according to these variations in the expanded configuration may have a spherical or ovoid, tetrahedral, cuboidal or semi spherical outer shape. Alternatively or additionally, the device may comprise one or more (e.g., one) circumferential belts instead of, or in addition to, the one or more circumferential threads.

In the above-discussed examples of the device, the resiliently deformable member (e.g., elastic or spring) is arranged between the arms. In variations, the resiliently deformable member may be arranged elsewhere. For example, the resiliently deformable member may be arranged on the sides of the arms, or inside the arms. In yet another example, the resiliently deformable member may form an arm of the device itself.

In the above-discussed examples of the device, the support elements (e.g., timers) are located inside the coupling heads, in particular in a hollow portion thereof. In variations, one or more support elements may be located elsewhere. For example, a resiliently deformable member (e.g., elastic or spring) may be made of two separate (i.e., initially disconnected) halves, the two halves being connected by a support element. When the support element degrades, the two halves disconnect.

In the above-discussed examples of the device, the arms are each arranged longitudinally (on axis X) alongside each other. In variations, one or more of the arms may be arranged differently. For example, the arms may be twisted together.

In the above-discussed examples, the optional load of API may be distinct and separate from the support elements (e.g., timers). Each support element may be integrally formed, and the load of the API may be elsewhere. Notably, the support elements are located inside the coupling heads, while the discussed load of the API is located between the coupling heads and the arms. The support element may comprise none of the API.

The above-discussed device may be adapted for a method of treating a condition which benefits from local dispensing of an API in the stomach or the ICV region of a subject suffering from said condition, comprising administering to said subject the device.

In specific implementations of the device 1000, 2000, or 2000′, preparation and assembly of the rigid device may be performed as follows. Main structural elements i.e., arms and heads, made of rigid material, for example by 3D printing or injection molding. The rigid elements can be made of biocompatible material that can optionally be biodegradable. Device arms and may be made by Stratasys® 3D printer rigid VERO or MEDIC medical grade or Visijet M3 Crystal Objet 3D printer material or cellulose acetate by injection molding. Each two arms may be hinged together with a nylon pin. The resiliently deformable member made of a silicone tube 55 A shore, for 3.1 mm diameter is pulled through one of the heads and first end and locked by a timer type A (e.g., made of HPMC-AS HG:MG:DBS ratio 8:2:1 by hot-melt extrusion. This 3-component composition includes HPMC-AS high grade (HG) soluble in pH above 7: HPMC-AS medium grade (MG) soluble in pH above 6.5 and DBS which is the plasticizer dibutyl sebacate. The resiliently deformable member is then pulled through a second head. Arms are then hinged to the heads via a designated arm-head locking structure. The resiliently deformable member is then stretched through the second head and locked by a timer type B (made of Eudragit® E PO by hot-melt extrusion). A circumferential thread is pulled through arms designated holes and tied at the end. Device dimensions in folded configuration based on this principle are about 27.5 mm in length and 8.8 mm in diameter, while in unfolded configuration diameter of about 22 mm. In example where the device carries API, holes are designed in both arm body and arm cover and before the arms are hinged together with a nylon pin, a tablet containing an API may be placed in each of the arm body cavities. Then, the arm cover may be clicked on the arm body/recipient to lock the tablet inside. Apertures may be designed in both arm body/recipient and the arm cover to allow drug release from arms. The locking member may be made of a more rigid material for example cellulose acetate. The device may then be placed into a container of 10 mm diameter.

In specific implementations where the resiliently deformable member is a spring, the spring may be stainless steel or Nitinol (nickel-titanium alloy) or other Biocompatible material suited for oral dosing and had integral hinge. Device dimensions in folded configuration based on this principle is about 27.5 mm in length and 8.5 mm in diameter, while in unfolded configuration diameter of about 22 mm.

In specific implementation of device 1000′ preparation and assembly of the rigid device may be performed as follows. Main structural elements i.e., arms and heads, are made of rigid material, for example by 3D printing or injection molding. The rigid elements can be made of biocompatible material that may optionally be biodegradable. Device arms and may be made by Stratasys® 3D printer rigid VERO or MEDIC medical grade or Visijet M3 Crystal Objet 3D printer material or cellulose acetate by injection molding. Each two arms may be hinged together with a nylon pin. The resiliently deformable member 1035′ made of a silicone tube 55A shore, for 1.5 mm diameter is pulled through the locking member and then through recess in head 1032′ (as illustrated in FIG. 5A). the other side of the locking member is placed into head 1034′ and a pin 1042′ made of a timer made of Eudragit® E PO by hot melt extrusion is passed through head to lock the timer in place. Arms are then hinged to the heads via a designated arm-head locking structure. Device dimensions in folded configuration based on this principle are about 28 mm in length and 8.4 mm in diameter, while in unfolded configuration diameter of about 22 mm. In examples where the device carries API, holes are designed in both arm body and arm cover and before the arms are hinged together with a nylon pin, a tablet containing an API may be placed in each of the arm body cavities. Then, the arm cover may be clicked on the arm body/recipient to lock the tablet inside. Apertures may be designed in both arm body/recipient and the arm cover to allow drug release from arms. The locking member may be made of a more rigid material for example cellulose acetate. The device may then be placed into a container of 9 mm diameter.

FIG. 10A illustrates retention testing equipment 200 that can be used in a method for assessing the capability of a device (such as the prototype device on the photo of FIG. 10B) to pass through the predetermined location of the GI tract using retention testing equipment. Basically, a method for assessing the capability of a device to pass through the predetermined location (e.g., by passing the pyloric valve or ileocecal valve) comprises: providing a retention test device configured to simulate the geometry of a standard region; placing a device in the retention test device upstream of a simulated pyloric valve or ICV; applying sequentially on the device increasing forces (e.g., substantially constant forces), each forces being preferably applied for a predetermined test time period (e.g., about 10 seconds), wherein said forces tend to pull the device out of the simulated pyloric valve or ICV, until an extraction force which is sufficient for extracting the device from the simulated pyloric valve or ICV within the test time period is reached; comparing the extraction force to a predetermined threshold (e.g., about 1.5 N) defining a minimal retention capability.

The retention test equipment 200 may include a retention test device 210 (also referred to as simulated pyloric valve or ileocecal valve) and a mounting support 250 for maintaining the retention test equipment vertically so that gravity tends to extract the device 100 through the simulated valve. The device 100 may be a device according to any of the embodiments and examples discussed above.

The retention test device 210 for simulating the geometry of standard valve region may generally include a funnel portion reproducing the narrowing of the valve. In more details, the test device 210 may comprise a first tubing portion 211 of a first diameter D1 joined at its connecting end to a narrowing truncated conical portion 212. An end of the truncated conical portion 212 opposed to the connecting end may be of a second diameter D2 smaller than D1. The narrowing conical portion 212 may be concentric to a second tubing portion 213 surrounding the narrowing conical portion 212. The second tubing portion 213 may be joined to the connecting end of the first tubing portion 211. In some embodiments, as illustrated, the first and second tubing portions 211 and 213 may be of a same diameter D1 and form a single tube. In some embodiments, the inner surface of the retention test device may be lubricated, for example with an edible oil, for example corn oil. In the following, the first tubing portion 211 and the narrowing portion 212 are also referred together as donor chamber and the second tubing portion 213 is also referred to as acceptor chamber. The first tubing portion 211 may be configured to simulate the predetermined location (the pyloric region when the predetermined location is the stomach and the terminal ileum region where the predetermined location is the ICV region of the small intestine) while the second tubing portion 213 to simulate the duodenum or cecum in the large intestine. The first diameter D1 of the first tubing portion 211 may be of about 20 to 35 mm, preferably of about 30 mm. A length of the first tubing portion 211 may be of about 100 mm. The conical narrowing portion 212 may be configured to simulate the geometry at the predetermined location. A length of the narrowing conical portion may be of about 12 mm. The second diameter D2 may be of about 10 to 20 mm, preferably of 16 mm. A length of the second tubing portion 213 may be of about 70 mm. The retention test device 210 may be made out of a rigid material.

The device 100 may be positioned at the connecting end of the first tubing portion 211 i.e., at the enlarged part of the funnel. The device 100 may be wrapped into a foil 230 on which increasing weights may be attached so as to pull the device out of the donor chamber into the acceptor chamber. The foil 230 may be preferably made of low density polyethylene with a thickness of about 10 microns. The foil may preferably have a rectangular shape of about 19 cm by 25 cm. An external surface of the foil 230 in contact with the test device 210 may be lubricated, preferably with corn oil.

The foil 230 may be sequentially attached to increasing weights in order to apply increasing forces on the device. The weights may be between about 0.098 N to 4.90 N. A predetermined extraction force threshold may correspond to a predetermined extraction weight threshold. The predetermined extraction weight threshold defining a (minimal) satisfying retention capability may be of about 0.98 N, 1.47 N, 2.45 N, 2.94 N or 3.43 N, 3.92 N, 4.41 N, 4.90 N or 5.88 N.

In some embodiments, the previously described method may be repeated several times and an average extraction force needed to extract the device out of the simulated region may be determined.

The previously described method may advantageously be used to determine the capability of a device in the expanded configuration to be retained in the predetermined location. In these embodiments, the device in the expanded configuration is considered to satisfy the retention capability if the measured extraction force is above the predetermined minimal retention threshold. In some embodiments, the method previously described may also be used to determine the capability of a device in the emptying configuration to be emptied i.e., to pass through the valve. In these embodiments, the comparing step may instead be: comparing the extraction force to a predetermined threshold defining a maximum acceptable retention capability. In these embodiments, the device in the emptying configuration is considered to satisfy the emptying capability if the measured extraction force is below the predetermined maximal retention threshold.

In particular, a method for determining the retention capability of a device includes: providing a retention test device mounted vertically, wherein the retention device includes a first tubing portion of a first diameter of about 30 mm joined at its connecting end to a narrowing truncated conical portion, an end of the truncated conical portion opposed to the connecting end being of a second diameter of about 16 mm, a length of the first tubing portion being of about 100 mm, a length of the narrowing truncated conical portion being of about 12 mm, the first tubing portion and narrowing truncated conical portion may be of rigid plastic; placing a device on the narrowing truncated conical portion; applying sequentially on the device increasing forces (e.g., substantially constant forces), each forces being preferably applied for about 10 seconds, wherein said forces tend to pull the device out of the narrowing truncated conical portion, until an extraction force which is sufficient for extracting the device from the narrowing truncated conical portion within 10 seconds is reached; comparing the extraction force to a predetermined threshold of about 1.0N or 1.5N or 2.0N.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

Disclosures of the publications cited throughout are hereby incorporated by reference in their entireties into this application in order to more fully describe the state of the art as of the date of the invention described herein.

This invention will be better understood by reference to the experimental details, which follow, but those skilled in the art will readily appreciate that the specific experiments detailed are only illustrative.

### Experimental 1: Unfolding and Retention Force Test and Shelf-Life

In vitro testing of devices according to the present disclosure were performed. During shelf-life, the devices were in the closed/folded configuration placed in a container such as a capsule enabling oral dosing. The devices are designed such that after dosing, when the capsule erodes, the devices expand/unfold and then maintain the unfolded configuration (resist folding back) to enable retention and trapping capabilities in the GI tract (e.g., ICV or stomach). In general, the device may respect a criterion of an unfolding force of 0.98-4.90 N to enable full unfolding in small intestine, more preferably 0.98-1.96 N to lower risk of unfolding and retention in esophagus and a retention force of >2.45 N, more preferably >3.92 N to maintain the device unfolded for local retention where defined. Thus, the following criteria were assessed: unfolding force between 0.98-4.90 N (preferably 0.98-1.96 N), retention force >2.45 N (even >4.90 N), long shelf-life (>1 week), and use of an as low amount of resiliently deformable member material as possible.

To evaluate if the devices met the criteria, test results obtained with different devices having a structure as disclosed and manufactured according to the assembly method detailed hereinabove. The material used for the devices where silicone shore 55 A for the resiliently deformable member, and a rigid material (Visij et M3 crystal) for the locking member. The dimensions for each tested device are provided in Table 1. A comparative test of unfolding force and retention force at t=0, after one week (1W) and after two months (2M) were conducted using the emptying test presented in reference to FIG. 10A in order to evaluate the unfolding force. D1 and D2 were 18 mm and 13 mm respectively to evaluate the unfolding force, while to evaluate the retention force D1 and D2 were 28 mm and 16 mm or 18 mm respectively. Results are summarized in Table 1 below. This experiment exemplifies the embodiments wherein the device comprising articulated arms enables achieving, long shelf life, high drug loading (>100 mg), high retention force, and a small size enabling easy swallowing.

TABLE 1
Comparative test results of unfolding force and retention force for devices according to the present disclosure.

| | | | | | | unfolding | | | | | | | | |
|---|---|---|---|---|---|---|---|---|---|---|---|---|---|---|
| | | Biasing assembly | | | | force (N) | | | | | | | | |
| Prototype | | folded | unfolded | | | (D 2 = 13 mm) | | | retention force | | | retention force | | |
| | Based | dimension | dimension | | | criteria: | | | (N) (D 2 = 16 mm) | | | (N) (D 2 = 18 mm) | | |
| | on | (Ø, length | (Ø in | | silicone | 0.98-4.90N | | | criteria >2.45N | | | criteria >2.45N | | |
| No. | device | in mm) | mm) | type | (mg) | t = 0 | 1 W | 6 M | t = 0 | 1W | 6 M | t = 0 | 1 W | 6 M |
| 1 | 1000 | 9.8, 29 | 24 | biasing | 55 | 2.35 | 1.86 | 1.86 | >4.90 | >4.90 | >4.90 | >4.90 | >4.90 | >4.90 |
| | | | | shortening | | | | | | | | | | |
| | | | | only | | | | | | | | | | |
| 2 | 2000 | 9.8, 29 | 24 | biasing | 40 | 1.96 | 1.76 | 1.76 | >4.90 | >4.90 | >4.90 | >4.90 | >4.90 | >4.90 |
| | | | | shortening + | | | | | | | | | | |
| | | | | straining | | | | | | | | | | |
| | | | | before | | | | | | | | | | |
| | | | | dosing | | | | | | | | | | |
| 3 | 1000 | 8.5, 28 | 22 | biasing | 30 | 1.47 | 1.47 | 1.47 | >4.90 | >4.90 | >4.90 | >4.90 | >4.90 | >4.90 |
| | | | | shortening | | | | | | | | | | |
| | | | | only | | | | | | | | | | |

The obtained results show that the biasing shortening and the straining before dosing allow to meet the criteria very well, with a long shelf-life since the forces are well maintained after time elapsed, even after two months.

### Experimental 2: Retention and Disassembly

A device according to the principles described above was made with three timer configurations as described below and was tested for retention and disassembly. A total of three devices were prepared for each timer configuration. Emptying test was applied as described in reference to FIG. 10A, while the medium used is as described in Tables 2a, 2b, 2c below.

Timer configuration 1: device 1000′ (FIG. 5) to simulate ICV retention with circumferential belt (1015, FIG. 1c). Timer was made of Eudragit® E PO by hot melt extrusion. Timer is designed to be activated when intestinal pH drops below pH 5 (e.g., by an external tablet that is trapped on device 1000′). The device was retained expanded configuration for 24 hr in pH 7.0 (4.90 N emptying test every 1 hr) while disassembled within 3 hr under emptying force of 1.47 N when exposed to pH 4.0. The results are given in Table 2a.

Timer configuration 2: device 1000′ (FIG. 5) to simulate ICV retention without circumferential belt (1015, FIG. 1c). Timer was made of HPMC-AS HG:MG:DBS (10:2:1) by hot melt extrusion. Timer was designed to gradually erode in intestinal pH. The device was retained in expanded configuration up to at least 8 hr in pH 7.0 (4.90 N emptying test every 1 hr) while when at 24 hr it disassembled and emptied under emptying force of 1.47 N. The results are given in Table 2b.

Timer configuration 3: device 1000′ (FIG. 5) to simulate gastric retention without circumferential belt (1015, FIG. 1c). Timer was made of Eudragit® E PO by hot melt extrusion. One side of the device timer housing was sealed (to lower the erosion rate of the timer). Timer was designed to gradually erode in gastric pH. Device was retained in expanded configuration up to at least 9 hr in pH 1.3 (4.90 N emptying test every 1 hr) while at 24 hr it disassembled and emptied under emptying force of 1.47 N. The results are given in Table 2c.

TABLE 2a
Timer configuration 1
Table 2a: timer configuration 1: Eudragit ® E PO*
in device 1000′ with trapping for ICV retention

| | | | | retention/ |
|---|---|---|---|---|
| Hr | medium | pH | status | emptying force |
| 0-24 | buffer phosphate | 7 | retained | 4.90N retention |
| | 0.02M | | | |
| 24-48 hr | buffer acetate | 4.0 | disassembled | 1.47N emptying |
| | | | within 3 hr | |

*Eudragit EPO consists of butyl methacrylate, dimethylaminoethyl methacrylate, methyl methacrylate copolymer (Evonik GmbH)

TABLE 2b
Timer configuration 2
Table 2b: timer configuration 2: HPMC AS in device
1000′ without trapping for ICV retention

| | | | | | retention/ |
|---|---|---|---|---|---|
| Hr | | medium | pH | status | emptying force |
| 0-3 | hr | buffer phosphate | 6.8 | retained | 4.90N retention |
| | | 0.015M | | | |
| 3-8 | hr | buffer phosphate | 7 | retained | 4.90N retention |
| | | 0.02M | | | |
| 24 | hr | buffer phosphate | 7 | disassembled | 1.47N emptying |
| | | 0.02M | | | |

TABLE 2c
Timer configuration 3
Table 2c: timer configuration 3: Eudragit ® E PO in device
1000′ without trapping for gastric retention

| | | | | |
|---|---|---|---|---|
| | | | | retention/ |
| hr | medium | pH | status | emptying force |

| | | | | | |
|---|---|---|---|---|---|
| 0-9 | hr | HCL | 1.3 | retained | 4.90N retention |
| 11 | hr | HCL | 1.3 | disassembled | 1.47N emptying |

### Experimental 3: Platforms Preparation

With reference to FIGS. 5A-K (of the device 1000′), the performance of the device in vivo, based on the principles of device 1000′ adapted for administration to a dog for temporary stomach retention was tested. GRDFs A, B, C and D were prepared having the features shown in Table 3. FIG. 10B shows an example of the prepared device.

TABLE 3
Features of the devices

| Features | device A | device B | device C | device D |
|---|---|---|---|---|
| Heads + arms + | Projet 3500 | Projet 3500 | Projet 3500 | Projet 3500 |
| locking member | HD 3D printer | HD 3D printer | HD 3D printer | HD 3D printer |
| (1036′ FIG. 5A) | From Visijet | From Visijet | From Visijet | From Visijet |
| | M3 Crystal | M3 Crystal | M3 Crystal | M3 Crystal |
| | material | material | material | material |
| resiliently | silicone 55A | silicone 55A | silicone 55A | silicone 55A |
| deformable | tube | tube | tube | tube |
| member (1035′, | 1.7 mm/0.5 mm | 1.7 mm/0.5 mm | 1.7 mm/0.5 mm | 1.7 mm/0.5 mm |
| FIG. 5A) | | | | |
| Circumferential | Visijet M3 | Visijet M3 | Visijet M3 | Visijet M3 |
| belt | Prolene ® | Prolene ® | Prolene ® | Prolene ® suture |
| | suture 5-0, | suture 5-0, | suture 5-0, | 5-0, |
| inner tablet | placebo, | placebo, | placebo, | placebo, |
| | barium based | barium based | barium based | barium based |
| | formulation | formulation | formulation | formulation |
| timer pin | timer type A, | timer type B, | timer type C, | timer type B, |
| (type, erosion | 48 hr | 24 hr | 12 hr | 24 hr |
| rate, in vitro) | | | | |
| Labeling | barium | barium | barium | barium |
| (platform and | | | | |
| inner formulation) | | | | |
| Dimensions in mm | 22.5, 68, ≈0.9 | 22.5, 68, ≈0.9 | 21, 60, ≈0.9 | 21, 60, ≈0.9 |
| (max diameter, max | | | | |
| circumference, | | | | |
| sphericity) | | | | |
| folded length/ | 29/8.5 | 29/8.5 | 27/8.5 | 27/8.5 |
| diameter (mm) | | | | |
| unfolding force | 1.47N | 1.47N | 1.47N | 1.47N |
| (D 2 = 13 mm) | | | | |
| folding force | >4.90N | >4.90N | >4.90N | >4.90N |
| (D 2 = 16 mm) | | | | |

Prolene® suture is a polypropylene suture (Ethicon). Devices A-D were manufactured and assembled as follows:

Device arms, heads and locking members were manufactured by 3D printing with a Objet 3500 printer using Visij et M3 Crystal biocompatible (medical grade) material. Each of the arms was labeled with a barium thread to enable identification in X-ray imaging. A placebo formulation was placed in each arm to enable monitoring formulation release in the stomach. Two formulations including Barium:Eudragit® E PO:MgOxide in distinct ratios (7:3:0, 7:3:1) were mixed, wetted with acetone and then injected by syringe into the platform arms (two arms with 7:3:0 ratio and two arms with 7:3:1 ratio) and left to dry for 24 hr in a hood. Alternatively, tablets which include placebo or API may be placed within one or more arm of the platform.

Each device was manufactured from 4 arms, where each arm was made of 2 sub-parts. Each two sub-parts of each arm were hinged together with a 0.5 mm nylon pin. A resiliently deformable biasing member (1035′ in FIG. 5A) made of biocompatible silicone 55 A tube 1.7/0.5 mm was first pulled through the rigid biasing member and through the cocking head (1032′ in FIG. 5A) as described above. The rigid biasing member extension was placed in the 2nd head (1034′ in FIG. 5A). A timer pin having 1.5 mm diameter was placed inside a silicone tube of 2/1.5 mm and the silicone tube was then threaded through the corresponding head to lock the rigid biasing member in the head. The tube was then cut from both sides to expose the timer pin. Timer pin type A (placed in platform A) was made of Eudragit® E PO:MgOxide:HPMC-AS MG (medium grade) at a ratio of 150:12.5:30, timer pin type B (placed in platforms B and D) was made of Eudragit® E PO. After placing time pin B inside head 1034′ it was sealed from one side with cellulose acetate 6% polymer, and timer pin type C (placed in platform C) was made of HPMC-AS MG+MgO at a ratio of 8:1. All pins were made by hot melt extrusion at about 140-160° C. The arms were then hinged into the two opposite heads' cavities. Device dimensions in the folded configuration are given in Table 3, above. The assembled device was subjected to in vitro unfolding and retention tests, as described in reference to FIG. 10A (unfolding test D2=13 mm, retention test D2=16 mm). Test results found acceptable. Results for unfolding and folding forces are summarized in Table 3, above.

In particular, the platforms described herein are spherical structures that enable GI retention with minimal potential surface pressure on surrounding tissue and potentially less adverse effects. The devices have a simple unfolding mechanism which occupies minimal dead volume allowing maximal free space for API (˜40%) in the folded (swallowing) configuration. The unfolding mechanism exhibits minimal strain of the elastic biasing member thereby enabling a long shelf life. When reaching the maximize size, in the open configuration the device is locked and enables structural resistance to a pressure of 1.5 kg/cm2

The sphericity of the unfolded devices is about 0.85-1 and each one has a circumference of less than about 70 mm, or about 60-70 mm. The maximal to minimal circumference in the unfolded configuration is about 1-1.2. The small timer mechanism design (1.5 mm diameter×6 mm length pin or 1.0 mm diameter×5 mm length pin) uses minimal space within the device thereby enabling higher drug loading. The timer as designed and described herein can resist frequent closing force of up to 1.5 Kg (14.7 N) while the platform is in the locked position, enabling gastric retention for long period of time (96 hours in vitro retention and more than 48 hr retention in dog stomach). Gastric retention requires physical resistance of the platform to gastric emptying forces, which are known to be even stronger in dog compared to human. The device may include a shock absorbing element within the biasing-timer system to better withstand impact of GI external forces. Once the timer mechanism has been released (e.g. eroded or degraded) the platform disassembles to small parts, each part less than about 20 mm×5 mm×3 mm enabling easy and safe emptying from the body. API may be loaded in one or more of the cavities in the arms of the platform. In some embodiments there are 8 cavities present in each platform. Loading of different API/release rate formulations (Formulation release in two rates demonstrated in beagle study).

### Experimental 4: Animal Study Using Dosing Platforms with Placebo Formulation

The study aimed to evaluate the performance of the GRDF systems in an animal model, beagle dogs. The tested performance features included administration, unfolding of the device in the stomach, retention of the device in the stomach for a predefined time, release of the loaded placebo formulation from the device, disassembly of the device and emptying of the device. Platforms A-D as described were labeled with barium to enable detection by X-ray imaging to track the location and status of the platform in the GI. A control capsule with 8×4 mm control “dummy tablets” were dosed to exemplify superior gastric retention of the platform while platform enabling emptying of objects. 3 timer rates were tested in the study to exemplify the ability to control the emptying time.

Study Design

Animals: 4 beagle dogs 11-13 kg each. Dogs were acclimated for about seven days before dosing. The animals were weighed and food consumption was assessed. 12 hr prior to each dosing, the dogs were fed with a conventional meal and then fasted. Fifteen (15) min prior to dosing, dogs were fed with a light meal (75 kcal). Dosing was done by hand followed by administration of 30 ml water. Dosing was performed by administering a platform in a capsule followed by a capsule comprising the 8 dummy tablets. After dosing, the dogs were fasted for an additional 8 hr and then fed a conventional meal. X ray (fluoroscopy) imaging was performed 5 min and 4, 8, 12, 24 and 48 hr after dosing while the dog was awake (in absence of anesthesia). Study dosing sequence and results are summarized in Table 4, below.

A total of 12 units were dosed in 4 cycles. The 1st dosing platform A, with a 48-72 hr timer was given to 4 dogs. The 2nd dosing platform B with a 24-48 hr timer was given to 4 dogs. The 3rd dosing platform C with a 12 hr timer was given to 2 dogs. The 4th dosing platform D with a 24-48 hr timer was given to 2 dogs. Table 4 outlines the study details.

TABLE 4

| Study details | | | | | | Device-results | | | Control tablets-results | | |
|---|---|---|---|---|---|---|---|---|---|---|---|
| | | | | Timer | # of | GR time | | GI | % | | |
| | | Folded | Unfolded | rate (hr in | dogs | hr/# | Platform | transit | tablets | % tablets | GI transit |
| dosing | platform | size (mm) | diam (mm) | vitro) | dosed | of dogs | GR >7 hr | time | GR >7 hr | GR >24 hr | time |
| 1 | A | 29 × 8.5 | 22.5 | 48-72 | 4 | 72, 2/2 | 100%, 4/4 | <24 hr | 47%, 15/32 | 0% | <24 hr |
| 2 | B | 29 × 8.5 | 22.5 | 24-48 | 4 | 48, 2/2 | 100%, 4/4 | <24 hr | 29%, 9/32 | 0% | <24 hr |
| 3 | C | 27 × 8.5 | 21 | 12 | 2 | 24, 2/2 | 100%, 2/2 | <24 hr | 60%, 19/32 | 0% | <24 hr |
| 4 | D | 27 × 8.5 | 21 | 24-48 | 2 | 48, 1/1 | 100%, 2/2 | <24 hr | 45%, 14/32 | 0% | <24 hr |

A few dogs vomited. Vomited cases were excluded from the gastroretentive (GR) statistics at the point of vomiting. Beagle dogs are known to be sensitive to vomiting (http://www.beaglepro.com/beagle-vomit-diarrhea).

Results:

Gastric Retention:

Gastric retention (GR) was 100% in all dogs that didn't vomit. GR was timer dependent (FIG. 11). * represents dogs that vomited and were excluded from the statistical analysis). The timer disassembly mechanisms worked 100% of the cases and achieved: 1st dosing timer 72 hr retention (2/2), 2nd and 4th timer 48 hr retention (3/3), 3rd dosing 24 hr retention 2/2. A total of 12/12 dosings achieved at least 7 hr GR.

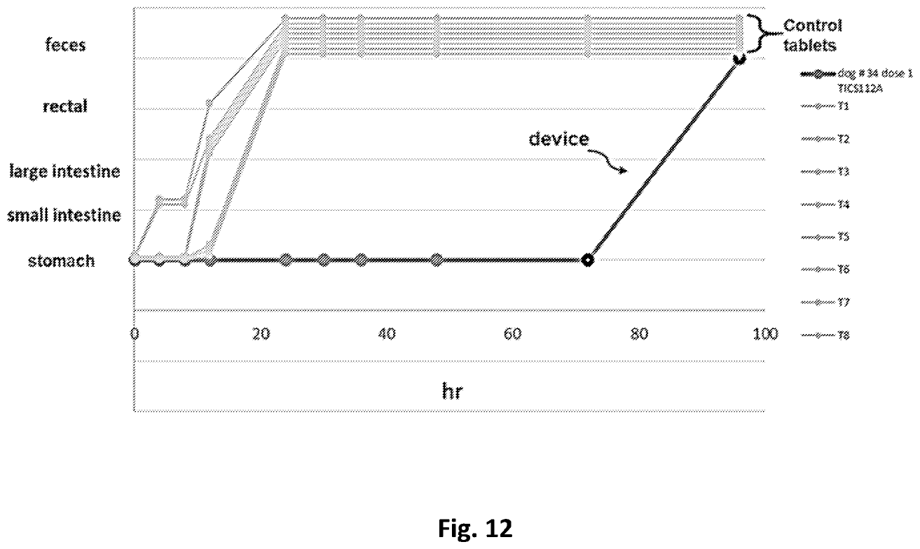

Gastric retention by platform was significantly higher compared than control tablets as demonstrated in FIGS. 11 and 12. Control tablet gastric emptying was comparable to literature data (http://www.2ndchance.info/rawdiet-Lalloo2012.pdf)

In vivo formulation release from the platform

Release rate of inner formulation from the platform was monitored by the degree of barium intensity in platform arms observed by fluoroscopy imaging (qualitative visual comparison of X-ray at a time interval compared to X ray imaging at T=5 min). In all cases, the formulation was almost completely eroded before disassembly and emptying. Exemplified imaging in one dog is provided in FIG. 12.

Transit Time Following Disassembly

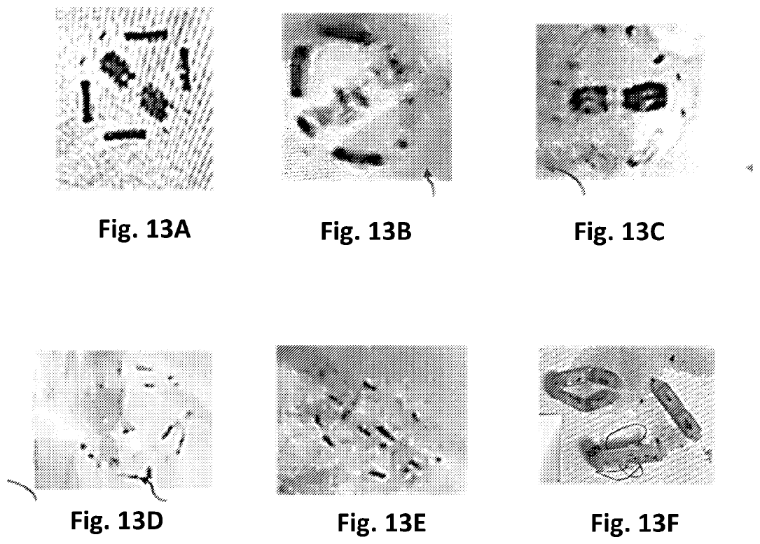

Following disassembly, the platform emptied from the stomach, transited through the intestine and emptied from the anus within an acceptable <24 hr. FIGS. 13A-13F are Xray images of an exemplary device (the timer used was: Eudragit® E PO:MgO:HPMC-AS MG in ratio of 150:12.5:30) and was followed in the GI tract of a beagle dog. FIG. 13A is the device at time 0, as administered to the stomach of the dog. The device is in expanded configuration in the stomach. FIG. 13B shows the device in the stomach at 8 hours post administration. FIG. 13C shows the device in the stomach at 24 hours post administration. FIG. 13D shows the device in the stomach at 48 hours post administration. FIG. 13E shows the device in the collapsing configuration in the small intestine at 72 hr post administration. FIG. 13F shows the parts of the disassembled device as recovered from the feces.

Safety

No safety events were identified or observed except for vomiting, which is common in beagle dogs. Food consumption, weight, animal behavior, bowl movement, feces texture were all normal. Food and control tablets were emptied in normal transit time.

### Experimental 5: Animal Study Dosing Platform with API Formulation

The study is conducted as described in Experimental 4. In a 1^{st} dosing, control conventional tablet (having no potential GR capabilities) of 20 mm/8 length/diameter with API labeled with barium, is dosed (LD). Blood samples are collected (e.g. at t=0, 1 hr, 2 hr, 3 hr, 4 hr, 5 hr, 6 hr, 8 hr, 12 hr, 24 hr, 36 hr, 48 hr to evaluate API concentration in plasma. X-ray imaging is performed at 5 min, 4 hr, 8 hr, 24 hr.

In the 2nd dosing, platform D with API is given. From dosing up to 48 hr, X-ray imaging is done (5 min, 4 hr, 8 hr, 12 hr, 24 hr, 48 hr) and blood samples are collected (e.g. at t=0, 1 hr, 2 hr, 3 hr, 4 hr, 5 hr, 6 hr, 8 hr, 12 hr, 24 hr, 36 hr, 48 hr) to evaluate API concentration in plasma.

Study Success Criteria

Gastric retention—The device is retained in the stomach in expanded configuration for at least 24 hr;

Plasma accumulation—higher plasma concentrations at 8, 12 and 24 hr and optionally at 36 and 48 hr compared to control conventional API tablet;

Platform disassembly—Platform disassembles at 48 hr and is emptied from the body after <48 hr after emptying from stomach.

Safety—No significant safety issues (health, food consumption, feces etc) The above discussed experimentals 3-5 indicate that the GRDF devices disclosed herein are effective and safe for temporary gastric retention and for the release of API in a fasted stomach for about 4 to 12 hr post dosing, preferably about 8 to 12 hr post dosing. The devices further collapse and exit the body once the timer is released.

1. A device for temporary residence at a predetermined location of the gastrointestinal tract of a subject, the device being configured to transfer from a closed configuration into an expanded configuration, the device comprising:
at least two flexible arms, each of the two flexible arms having a first end and a second end;
first and second coupling heads, wherein the first end of each of the two flexible arms is coupled to the first coupling head and the second end of each of the two flexible arms is coupled to the second coupling head;
a resiliently deformable member configured to force the first and second coupling heads together to bend the at least two flexible arms thereby biasing the device in the expanded configuration; and
a locking assembly configured to maintain the device in the expanded configuration upon transfer of the device into the expanded configuration,
the device being configured in the expanded configuration, when the device is positioned at the predetermined location of the gastrointestinal tract, to be retained at the predetermined location of the gastrointestinal tract, and
the device being configured to transfer from the expanded configuration into an emptying configuration, the device being configured to move past the predetermined location of the gastrointestinal tract in the emptying configuration.

2. The device of claim 1, wherein the locking assembly comprises a locking member arranged between the first and second coupling heads and having two ends, at least one end of the locking member being free in the closed configuration, the locking member being configured for each free end to engage its respective coupling head when the device transfers from the closed configuration into the expanded configuration.

3. The device of claim 2, wherein the respective coupling head of at least one free end of the locking member comprises a recess configured to receive the free end of the locking member.

4-5. (canceled)

6. The device of claim 1, wherein the closed configuration has a storage sub-configuration and a dosing sub-configuration, the resiliently deformable member having two ends each configured to pull a respective coupling head, at least one end of the resiliently deformable member being free in the storage sub-configuration, the resiliently deformable member being configured to be stretched thereby transferring the device into the dosing sub-configuration.

7. The device of claim 6, wherein in the storage sub-configuration, the resiliently deformable member is configured to be stretched by pulling each free end via a respective lead.

8-10. (canceled)

11. The device of claim 7, wherein the respective lead has one or more weak points for cutting the lead after the free end is attached to its respective coupling head and the lead continues to be pulled.

12. The device of claim 7, wherein at least one free end of the resiliently deformable member comprises an extension configured for protruding out of its respective coupling head after said end is attached to its respective coupling head, the resiliently deformable member comprising a cutting zone at the basis of the extension for cutting the extension after said end is attached to its respective coupling head and the lead continues to be pulled.

13. The device of claim 7, wherein the respective lead is configured to be attached to a packaging such that when removing the device out of the packaging, the respective lead pulls its respective free end of the resiliently deformable member.

14-15. (canceled)

16. The device of claim 1, further comprising:
a flexible belt engaging each of the at least two flexible arms, wherein the belt is configured to tension as the device transfers from the closed configuration to the expanded configuration.

17-20. (canceled)

21. A device for temporary residence at a predetermined location of the gastrointestinal tract of a subject, the device being configured to transfer from a closed configuration into an expanded configuration, wherein:
when the device is in the expanded configuration and positioned at the predetermined location of the gastrointestinal tract, the device is configured to be retained at the predetermined location of the gastrointestinal tract,
when the device transfers from the expanded configuration into an emptying configuration, the device is configured to move past the predetermined location of the gastrointestinal tract,
the device further comprises a biasing assembly having a strained state and a relaxed state, the biasing assembly being configured, when the device is in the closed configuration, to transfer from the relaxed state into the strained state, and
the device being configured to transfer from the closed configuration into the expanded configuration when the biasing assembly is in the strained state.

22. The device of claim 21, wherein the device further comprises a straining assembly configured to transfer the biasing assembly from the relaxed state into the strained state upon the device being removed from a packaging.

23-25. (canceled)

26. The device of claim 21, wherein the biasing assembly comprises a resiliently deformable member having two ends each configured to pull a respective part of the device thereby biasing the device in the expanded configuration, at least one end of the resiliently deformable member being free in the relaxed state, the resiliently deformable member being configured to be stretched thereby transferring the biasing assembly from the relaxed state into the strained state.

27-39. (canceled)

40. The device of claim 1, further comprising a container to temporarily at least partially enclose the device in the closed configuration, wherein the container is configured to degrade in environment conditions of the predetermined location of the gastrointestinal tract.

41-43. (canceled)

44. The device of claim 1, further comprising a trigger assembly configured to cause the device to transfer from the expanded configuration into the emptying configuration.

45. The device of claim 1, wherein the device is configured to release of an active pharmaceutical ingredient at the predetermined location of the gastrointestinal tract in the expanded configuration.

46. (canceled)

47. The device of claim 1, wherein the predetermined location of the gastrointestinal tract is the stomach, the device being configured to be retained in the stomach in the expanded configuration, and to pass through the pyloric valve in the emptying configuration.

48. The device of claim 1, wherein the predetermined location of the gastrointestinal tract is the ileocecal valve, the device being configured to be retained at the ileocecal valve while allowing chyme flow in the expanded configuration, and to pass through the ileocecal valve in the emptying configuration.

49. (canceled)

50. The device of claim 48, wherein the device comprises a trapping assembly configured for blocking a ingestible object while allowing chyme flow when the device is positioned at the ileocecal valve and in the expanded configuration.

51-52. (canceled)

53. An oral dosage form for administration to a subject, the dosage form comprising an amount of an emptying agent within the device of claim 1, wherein the amount of the emptying agent is sufficient to cause at least one activation environmental condition to be reached at the predetermined location, thereby causing the device to transfer from the expanded configuration into the emptying configuration.

54. An oral dosage form for administration to a subject suffering from a condition which may benefit from local dispensing at the ileocecal valve region an active pharmaceutical ingredient (API) when the device of claim 1 is positioned at the ileocecal valve of the subject in the expanded configuration, wherein the dosage form comprises a coating for inhibiting release of said API in the gastric environment and enabling release of said API in the ileocecal valve region, wherein external dimensions of the dosage form enable the dosage form to be blocked by the device when reaching the ileocecal valve region.

55-58. (canceled)
